(19) United States
(12) Patent Application Publication
(10) Pub. No.: US 2025/0369973 A1
(43) Pub. Date: Dec. 4, 2025

(54) PROGNOSTIC MARKERS

(71) Applicants: BAKER HEART AND DIABETES INSTITUTE, Melbourne, Victoria (AU); LIFEHOUSE AUSTRALIA, Camperdown, New South Wales (AU); NSW HEALTH PATHOLOGY, Newcastle, New South Wales (AU); GARVAN INSTITUTE OF MEDICAL RESEARCH, Darlinghurst, New South Wales (AU); SYDNEY LOCAL HEALTH DISTRICT, Camperdown, New South Wales (AU)

(72) Inventors: Lisa Glen HORVATH, Camperdown, NSW (AU); Tahlia Judith SCHEINBERG, Camperdown, NSW (AU); Peter John MEIKLE, Lower Plenty, Victoria (AU); Hui Ming LIN, Massagno (CH); David Richmond SULLIVAN, Camperdown, NSW (AU)

(21) Appl. No.: 19/108,131

(22) PCT Filed: Sep. 1, 2023
(86) PCT No.: PCT/AU2023/050849

§ 371 Date: Feb. 28, 2025

(30) Foreign Application Priority Data
Sep. 2, 2022 (AU) 2022902527

(51) Int. Cl. G01N 33/574; A61K 31/4409; C07K 16/40

(52) U.S. Cl. CPC G01N 33/57434; A61K 31/4409; C07K 16/40; C07K 2317/21; C07K 2317/76; G01N 2405/04; G01N 2405/08

(57) ABSTRACT

The invention relates to methods for determining or predicting prognosis for and survival of a subject with prostate cancer, the methods comprising determining the level of at least three lipid biomarkers in a biological sample obtained from the subject, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0), and to compositions and kits for use in the methods.

### FIELD OF THE INVENTION

This invention relates generally to biomarkers for prostate cancer. More particularly the invention relates to lipid biomarkers and their use in kits, compositions and methods for determining or predicting prognosis for and survival of a subject with prostate cancer, particularly metastatic castration-resistant prostate cancer.

### BACKGROUND OF THE INVENTION

Prostate cancer is one of the most common cancers and is the fifth leading cause of cancer-related death in males worldwide. Although advances have recently been made in treatments for prostate cancer, particularly metastatic castration-resistant prostate cancer, due to the development of androgen receptor signalling inhibitor and other therapies, the utility of these therapies is limited due to the development of resistance. In this regard, intrinsic resistance affects approximately 20-30% of subjects and acquired resistance eventually develops for all patients on these therapies. As such, there is a need for new therapies for the treatment of prostate cancer and tailored therapeutic regimes which can avoid the development of resistance.

Sphingolipids, such as ceramides, are known to regulate various biological processes including cell growth. Ceramides are known to promote tumour growth and metastasis through the ceramide-sphingosine-1-phosphate signalling axis in cancer cells and immune cells. It has recently been found that elevated circulating levels of sphingolipids were associated with shorter metastasis-free survival in localised prostate cancer, earlier androgen-deprivation therapy failure in metastatic hormone-sensitive prostate cancer, and shorter progression-free survival or overall survival after administration of docetaxel or an androgen receptor signalling inhibitor in metastatic castration-resistant prostate cancer patients. As such, sphingolipids, play an important role in the progression of prostate cancer and are a viable target for prostate cancer treatments. However, as not all patients will benefit from such treatments, methods for determining subjects having prostate cancer that will benefit from therapies targeting lipids, such as sphingolipids, are desired.

A circulating three lipid signature (3LS) which is associated with a shorter overall survival in metastatic castration-resistant prostate cancer patients has previously been described. However, the high-throughput LC-MS methods used to detect the lipids, ceramide (d18:1/24:1), sphingomyelin (d18:2/16:0) and phosphatidylcholine (16:0/16:0), has a number of drawbacks, including the lack of assay standardisation and validation via regulatory or industry standards, and a lack of reproducibility of LC-MS methods between laboratories. Furthermore, the assay for detecting the three lipid signature can only be performed retrospectively, and cannot be used to prospectively identify males with prostate cancer who may benefit from therapies targeting lipids. Accurate and reproducible methods for determining the prognosis and survival of subjects with prostate cancer are desired.

### SUMMARY OF THE INVENTION

The present invention is predicated in part on the discovery that abnormal lipid levels can be used to identify subjects with prostate cancer that are likely to be resistant to conventional treatment, for example, with chemotherapy or treatment with an androgen receptor signalling inhibitor and, accordingly, have a poor prognosis and reduced overall survival. Accordingly, the inventors have conceived that levels of specific combinations of lipids (e.g. ceramides) may be used in kits, compositions and methods for determining or predicting prognosis for and survival of a subject with prostate cancer, particularly metastatic castration-resistant prostate cancer.

In one aspect, there is provided a method for determining an indicator used in determining prognosis for a subject with prostate cancer, the method comprising, consisting or consisting essentially of:
a) determining the level of at least three lipid biomarkers in a biological sample obtained from the subject, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0); and
b) determining the indicator using the biomarker levels.

In some embodiments, the prostate cancer is castration-resistant prostate cancer; especially a metastatic cancer.

In particular embodiments, the lipid biomarkers are selected from the group consisting of ceramide (d18:1/18:0), ceramide (d18:1/22:0), ceramide (d18:1/24:0), ceramide (d18:1/24:1), ceramide (d20:1/24:1) and phosphatidylcholine (16:0/16:0); especially wherein the lipid biomarkers are selected from the group consisting of ceramide (d18:1/18:0), ceramide (d18:1/22:0), ceramide (d18:1/24:0), ceramide (d18:1/24:1) and ceramide (d20:1/24:1). In specific embodiments, the lipid biomarkers are ceramide (d18:1/18:0), ceramide (d18:1/24:0) and ceramide (d18:1/24:1).

In further embodiments, step a) further comprises determining the level of one or more biomarkers selected from the group consisting of total cholesterol, triglycerides and high-density lipoprotein; especially the level of one or more biomarkers selected from the group consisting of total cholesterol and triglycerides. In specific embodiments, step a) further comprises determining the level of total cholesterol and triglycerides.

In particular embodiments, step a) comprises determining the level of ceramide (d18:1/18:0), ceramide (d18:1/24:0), ceramide (d18:1/24:1), total cholesterol and triglycerides.

In some embodiments, the biological sample is blood, serum or plasma; especially plasma.

In some embodiments, a poor prognosis comprises disease progression and/or death from disease within about 18 months from commencement of chemotherapy or treatment with an androgen receptor signalling inhibitor. A good prognosis, in some embodiments, comprises no disease progression and/or no death from disease within about 18 months from commencement of chemotherapy or treatment with an androgen receptor signalling inhibitor.

In specific embodiments, the indicator indicates a likelihood of a poor prognosis if: the level of ceramide (d18:1/18:0) is higher than in control biological samples obtained from a reference population of subjects with prostate cancer with a favourable outcome; the level of ceramide (d18:1/22:0) is lower than in control biological samples obtained from a reference population of subjects with prostate cancer with a favourable outcome; the level of ceramide (d18:1/24:0) is lower than in control biological samples obtained from a reference population of subjects with prostate cancer with a favourable outcome; the level of ceramide (d18:1/24:1) is higher than in control biological samples obtained from a reference population of subjects with prostate cancer with a favourable outcome; the level of ceramide (d20:1/24:0) is lower than in control biological samples obtained from a reference population of subjects with prostate cancer with a favourable outcome; the level of ceramide (d20:1/24:1) is higher than in control biological samples obtained from a reference population of subjects with prostate cancer with a favourable outcome; and/or the level of phosphatidylcholine (16:0/16:0) is higher than in control biological samples obtained from a reference population of subjects with prostate cancer with a favourable outcome. In some embodiments, step a) further comprises determining the level of one or more biomarkers selected from the group consisting of total cholesterol, triglycerides and high-density lipoprotein, and the indicator indicates a likelihood of a poor prognosis if: the level of total cholesterol is lower than in control biological samples obtained from a reference population of subjects with prostate cancer with a favourable outcome; the level of triglycerides is lower than in control biological samples obtained from a reference population of subjects with prostate cancer with a favourable outcome; and/or the level of high-density lipoprotein is lower than in control biological samples obtained from a reference population of subjects with prostate cancer with a favourable outcome.

In some embodiments, the method further comprises applying a function to biomarker levels to yield at least one functionalised biomarker level and determining the indicator using the at least one functionalised biomarker level. In particular embodiments, the function includes at least one of: (a) multiplying biomarker levels; (b) dividing biomarker levels; (c) adding biomarker levels; and (d) subtracting biomarker levels.

In some embodiments, the method further comprises combining the biomarker levels and/or functionalised biomarker levels to provide a composite score and determining the indicator using the composite score. In particular embodiments, the biomarker levels and/or functionalised biomarker levels are combined by adding, multiplying, subtracting, and/or dividing biomarker levels and/or functionalised biomarker levels.

In particular embodiments, the method further comprises analysing the biomarker levels, functionalised biomarker level(s) or composite score with reference to a corresponding reference biomarker level range or cut-off levels, functionalised biomarker level range or cut-off levels, or reference composite score range or cut-off scores, to determine the indicator.

In specific embodiments, the method comprises determining the indicator by dividing the level of ceramide (d18:1/24:0) by the level of ceramide (d18:1/24:1), and adding this value to the level of ceramide (d18:1/18:0), the level of total cholesterol, and the level of triglycerides, or functionalised levels of any of the foregoing levels. In particular embodiments, the method comprises determining the indicator using Formula I:

$$[ 10.0506 \times the level of ceramide ( d 18 : 1 / 18 : 0 ) in mg / L ] + [ - 0.2783 \times the level of total cholesterol in mmol / L ] + [ - 0.224 \times the level of triglycerides in mmol / L ] + [ - 0.2979 \times ( the level of ceramide ( d 18 : 1 / 24 : 0 ) in mg / L / the level of ceramide ( d 18 : 1 / 24 : 1 ) in mg / L ) ] \qquad Formula I$$

In specific embodiments, a score of greater than or equal to −1.1903 calculated using Formula I indicates a likelihood of a poor prognosis, and a score of less than −1.1903 calculated using Formula I indicates a likelihood of a good prognosis.

In some embodiments, the subject has undergone, is undergoing or is commencing a treatment regimen for treating prostate cancer, such as chemotherapy and/or administration of an androgen receptor signalling inhibitor.

In some embodiments, a subject determined to have a poor prognosis is administered a treatment for prostate cancer, such as a lipid targeted therapy, including administration of evolocumab or opaganib.

In some embodiments, the levels of the biomarkers are quantitative levels. In some embodiments, the levels of the biomarkers are determined using mass spectrometry; especially wherein the levels of the biomarkers are determined using absolute quantification.

In another aspect, there is provided a method for determining or predicting prognosis for a subject with prostate cancer, the method comprising, consisting or consisting essentially of:
a) determining the level of at least three lipid biomarkers in a biological sample obtained from the subject, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0);
b) comparing the level of the biomarkers to the respective levels of the corresponding biomarkers in a reference sample; and
c) determining or predicting prognosis for the subject based on the determined levels of the biomarkers and said comparison.

In some embodiments, the reference sample is a control biological sample obtained from a reference population of subjects with prostate cancer having a favourable outcome.

In particular embodiments, predicting prognosis comprises predicting subject survival.

In some embodiments, the prostate cancer is castration-resistant prostate cancer; especially wherein the prostate cancer is a metastatic cancer.

In some embodiments, the lipid biomarkers are selected from the group consisting of ceramide (d18:1/18:0), ceramide (d18:1/22:0), ceramide (d18:1/24:0), ceramide (d18:1/24:1), ceramide (d20:1/24:1) and phosphatidylcholine (16:0/16:0); especially wherein the lipid biomarkers are selected from the group consisting of ceramide (d18:1/18:0), ceramide (d18:1/22:0), ceramide (d18:1/24:0), ceramide (d18:1/24:1) and ceramide (d20:1/24:1). In specific embodiments, the lipid biomarkers are ceramide (d18:1/18:0), ceramide (d18:1/24:0) and ceramide (d18:1/24:1).

In some embodiments, step a) further comprises determining the level of one or more biomarkers selected from the group consisting of total cholesterol, triglycerides and high-density lipoprotein; especially wherein step a) further comprises determining the level of one or more biomarkers selected from the group consisting of total cholesterol and triglycerides. In specific embodiments, step a) further comprises determining the level of total cholesterol and triglycerides.

In specific embodiments, step a) comprises determining the level of ceramide (d18:1/18:0), ceramide (d18:1/24:0), ceramide (d18:1/24:1), total cholesterol and triglycerides.

In some embodiments, the biological sample is blood, serum or plasma; especially plasma.

In some embodiments, a poor prognosis comprises disease progression and/or death from disease within about 18 months from commencement of chemotherapy or treatment with an androgen receptor signalling inhibitor. In such embodiments, a good prognosis comprises no disease progression and/or no death from disease within about 18 months from commencement of chemotherapy or treatment with an androgen receptor signalling inhibitor.

In particular embodiments, the reference sample is a control biological sample obtained from a reference population of subjects with prostate cancer having a favourable outcome, and the subject has a likelihood of a poor prognosis if: the level of ceramide (d18:1/18:0) is higher than in the reference sample; the level of ceramide (d18:1/22:0) is lower than in the reference sample; the level of ceramide (d18:1/24:0) is lower than in the reference sample; the level of ceramide (d18:1/24:1) is higher than in the reference sample; the level of ceramide (d20:1/24:0) is lower than in the reference sample; the level of ceramide (d20:1/24:1) is higher than in the reference sample; and/or the level of phosphatidylcholine (16:0/16:0) is higher than in the reference sample. In further embodiments, step a) further comprises determining the level of one or more biomarkers selected from the group consisting of total cholesterol, triglycerides and high-density lipoprotein, the reference sample is a control biological sample obtained from a reference population of subjects with prostate cancer having a favourable outcome and the subject has a likelihood of a poor prognosis if: the level of total cholesterol is lower than in the reference sample; the level of triglycerides is lower than in the reference sample; and/or the level of high-density lipoprotein is lower than in the reference sample.

In some embodiments, the method comprises determining the prognosis for the subject by dividing the level of ceramide (d18:1/24:0) by the level of ceramide (d18:1/24:1), and adding this value to the level of ceramide (d18:1/18:0), the level of total cholesterol, and the level of triglycerides, or functionalised levels of any of the foregoing levels.

In specific embodiments, the method comprises determining the prognosis for the subject using Formula I:

$$[ 10.0506 \times the level of ceramide ( d 18 : 1 / 18 : 0 ) in mg / L ] + [ - 0.2783 \times the level of total cholesterol in mmol / L ] + [ - 0.224 \times the level of triglycerides in mmol / L ] + [ - 0.2979 \times ( the level of ceramide ( d 18 : 1 / 24 : 0 ) in mg / L / the level of ceramide ( d 18 : 1 / 24 : 1 ) in mg / L ) ] \qquad Formula I$$

In such embodiments, a score of greater than or equal to −1.1903 calculated using Formula I indicates a likelihood of a poor prognosis, and a score of less than −1.1903 calculated using Formula I indicates a likelihood of a good prognosis.

In some embodiments, the subject has undergone, is undergoing or is commencing a treatment regimen for treating prostate cancer, such as chemotherapy and/or administration of an androgen receptor signalling inhibitor.

In some embodiments, a subject determined to have a poor prognosis is administered a treatment for prostate cancer, such as a lipid targeted therapy, for example, evolocumab or opaganib.

In some embodiments, the levels of the biomarkers are quantitative levels.

In particular embodiments, the levels of the biomarkers are determined using mass spectrometry; especially using absolute quantification.

In a further aspect, there is provided a method for determining or predicting the likelihood of survival of a subject with prostate cancer, the method comprising, consisting or consisting essentially of:
a) determining the level of at least three lipid biomarkers in a biological sample obtained from the subject, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0); and
b) comparing the level of the biomarkers to the respective levels of the corresponding biomarkers in a reference sample; and
c) determining or predicting the likelihood of survival of the subject based on the determined levels of the biomarkers and said comparison.

In some embodiments, the reference sample is a control biological sample obtained from a reference population of subjects with prostate cancer having a favourable outcome.

In particular embodiments, the prostate cancer is castration-resistant prostate cancer; especially a metastatic cancer.

In some embodiments, the lipid biomarkers are selected from the group consisting of ceramide (d18:1/18:0), ceramide (d18:1/22:0), ceramide (d18:1/24:0), ceramide (d18:1/24:1), ceramide (d20:1/24:1) and phosphatidylcholine (16:0/16:0); especially wherein the lipid biomarkers are selected from the group consisting of ceramide (d18:1/18:0), ceramide (d18:1/22:0), ceramide (d18:1/24:0), ceramide (d18:1/24:1) and ceramide (d20:1/24:1). In particular embodiments, the lipid biomarkers are ceramide (d18:1/18:0), ceramide (d18:1/24:0) and ceramide (d18:1/24:1).

In some embodiments, step a) further comprises determining the level of one or more biomarkers selected from the group consisting of total cholesterol, triglycerides and high-density lipoprotein; especially the level of one or more biomarkers selected from the group consisting of total cholesterol and triglycerides. In particular embodiments, step a) further comprises determining the level of total cholesterol and triglycerides.

In particular embodiments, step a) comprises determining the level of ceramide (d18:1/18:0), ceramide (d18:1/24:0), ceramide (d18:1/24:1), total cholesterol and triglycerides.

In some embodiments, the biological sample is blood, serum or plasma; especially plasma.

In some embodiments, survival comprises no death from disease within about 18 months from commencement of chemotherapy or treatment with an androgen receptor signalling inhibitor.

In particular embodiments, the reference sample is a control biological sample obtained from a reference population of subjects with prostate cancer having a favourable outcome and the subject has a likelihood of non-survival if: the level of ceramide (d18:1/18:0) is higher than in the reference sample; the level of ceramide (d18:1/22:0) is lower than in the reference sample; the level of ceramide (d18:1/24:0) is lower than in the reference sample; the level of ceramide (d18:1/24:1) is higher than in the reference sample; the level of ceramide (d20:1/24:0) is lower than in the reference sample; the level of ceramide (d20:1/24:1) is higher than in the reference sample; and/or the level of phosphatidylcholine (16:0/16:0) is higher than in the reference sample. In particular embodiments, step a) further comprises determining the level of one or more biomarkers selected from the group consisting of total cholesterol, triglycerides and high-density lipoprotein, and the subject has a likelihood of non-survival if: the level of total cholesterol is lower than in the reference sample; the level of triglycerides is lower than in the reference sample; and/or the level of high-density lipoprotein is lower than in the reference sample.

In some embodiments, the method comprises determining the likelihood of survival by dividing the level of ceramide (d18:1/24:0) by the level of ceramide (d18:1/24:1), and adding this value to the level of ceramide (d18:1/18:0), the level of total cholesterol, and the level of triglycerides, or functionalised levels of any of the foregoing levels. In particular embodiments, the method comprises determining the likelihood of survival using Formula I:

$$[ 10.0506 \times the level of ceramide ( d 18 : 1 / 18 : 0 ) in mg / L ] + [ - 0.2783 \times the level of total cholesterol in mmol / L ] + [ - 0.224 \times the level of triglycerides in mmol / L ] + [ - 0.2979 \times ( the level of ceramide ( d 18 : 1 / 24 : 0 ) in mg / L / the level of ceramide ( d 18 : 1 / 24 : 1 ) in mg / L ) ] \qquad Formula I$$

In specific embodiments, a score of greater than or equal to −1.1903 calculated using Formula I indicates a likelihood of non-survival, and a score of less than −1.1903 calculated using Formula I indicates a likelihood of survival.

In some embodiments, the subject has undergone, is undergoing or is commencing a treatment regimen for treating prostate cancer, such as chemotherapy and/or administration of an androgen receptor signalling inhibitor.

In some embodiments, a subject determined to have a likelihood of non-survival is administered a treatment for prostate cancer, such as a lipid targeted therapy, for example, evolocumab or opaganib.

In some embodiments, the levels of the biomarkers are quantitative levels. In particular embodiments, the levels of the biomarkers are determined using mass spectrometry; especially using absolute quantification.

In yet another aspect, there is provided a method for determining an indicator used in determining a likelihood of survival of a subject with prostate cancer, the method comprising, consisting or consisting essentially of:
a) determining the level of at least three lipid biomarkers in a biological sample obtained from the subject, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0); and
b) determining the indicator using the biomarker levels.

In some embodiments, the prostate cancer is castration-resistant prostate cancer; especially a metastatic cancer.

In particular embodiments, the lipid biomarkers are selected from the group consisting of ceramide (d18:1/18:0), ceramide (d18:1/22:0), ceramide (d18:1/24:0), ceramide (d18:1/24:1), ceramide (d20:1/24:1) and phosphatidylcholine (16:0/16:0); especially wherein the lipid biomarkers are selected from the group consisting of ceramide (d18:1/18:0), ceramide (d18:1/22:0), ceramide (d18:1/24:0), ceramide (d18:1/24:1) and ceramide (d20:1/24:1). In specific embodiments, the lipid biomarkers are ceramide (d18:1/18:0), ceramide (d18:1/24:0) and ceramide (d18:1/24:1).

In some embodiments, step a) further comprises determining the level of one or more biomarkers selected from the group consisting of total cholesterol, triglycerides and high-density lipoprotein; especially the level of one or more biomarkers selected from the group consisting of total cholesterol and triglycerides. In specific embodiments, step a) further comprises determining the level of total cholesterol and triglycerides.

In particular embodiments, step a) comprises determining the level of ceramide (d18:1/18:0), ceramide (d18:1/24:0), ceramide (d18:1/24:1), total cholesterol and triglycerides.

In some embodiments, the biological sample is blood, serum or plasma; especially plasma.

In some embodiments, survival comprises no death from disease within about 18 months from commencement of chemotherapy or treatment with an androgen receptor signalling inhibitor.

In some embodiments, the indicator indicates a likelihood of non-survival if: the level of ceramide (d18:1/18:0) is higher than in control biological samples obtained from a reference population of subjects with prostate cancer with a favourable outcome; the level of ceramide (d18:1/22:0) is lower than in control biological samples obtained from a reference population of subjects with prostate cancer with a favourable outcome; the level of ceramide (d18:1/24:0) is lower than in control biological samples obtained from a reference population of subjects with prostate cancer with a favourable outcome; the level of ceramide (d18:1/24:1) is higher than in control biological samples obtained from a reference population of subjects with prostate cancer with a favourable outcome; the level of ceramide (d20:1/24:0) is lower than in control biological samples obtained from a reference population of subjects with prostate cancer with a favourable outcome; the level of ceramide (d20:1/24:1) is higher than in control biological samples obtained from a reference population of subjects with prostate cancer with a favourable outcome; and/or the level of phosphatidylcholine (16:0/16:0) is higher than in control biological samples obtained from a reference population of subjects with prostate cancer with a favourable outcome. In further embodiments, step a) further comprises determining the level of one or more biomarkers selected from the group consisting of total cholesterol, triglycerides and high-density lipoprotein, and the indicator indicates a likelihood of non-survival if: the level of total cholesterol is lower than in control biological samples obtained from a reference population of subjects with prostate cancer with a favourable outcome; the level of triglycerides is lower than in control biological samples obtained from a reference population of subjects with prostate cancer with a favourable outcome; and/or the level of high-density lipoprotein is lower than in control biological samples obtained from a reference population of subjects with prostate cancer with a favourable outcome.

In some embodiments, the method further comprises applying a function to biomarker levels to yield at least one functionalised biomarker level and determining the indicator using the at least one functionalised biomarker level; especially wherein the function includes at least one of: (a) multiplying biomarker levels; (b) dividing biomarker levels; (c) adding biomarker levels; and (d) subtracting biomarker levels. In some embodiments, the method further comprises combining the biomarker levels and/or functionalised biomarker levels to provide a composite score and determining the indicator using the composite score; especially wherein the biomarker levels and/or functionalised biomarker levels are combined by adding, multiplying, subtracting, and/or dividing biomarker levels and/or functionalised biomarker levels.

In some embodiments, the method further comprises analysing the biomarker levels, functionalised biomarker level(s) or composite score with reference to a corresponding reference biomarker level range or cut-off levels, functionalised biomarker level range or cut-off levels, or reference composite score range or cut-off scores, to determine the indicator.

In particular embodiments, the method comprises determining the indicator by dividing the level of ceramide (d18:1/24:0) by the level of ceramide (d18:1/24:1), and adding this value to the level of ceramide (d18:1/18:0), the level of total cholesterol, and the level of triglycerides, or functionalised levels of any of the foregoing levels.

In specific embodiments, the method comprises determining the indicator using Formula I:

$$[ 10.0506 \times the level of ceramide ( d 18 : 1 / 18 : 0 ) in mg / L ] + [ - 0.2783 \times the level of total cholesterol in mmol / L ] + [ - 0.224 \times the level of triglycerides in mmol / L ] + [ - 0.2979 \times ( the level of ceramide ( d 18 : 1 / 24 : 0 ) in mg / L / the level of ceramide ( d 18 : 1 / 24 : 1 ) in mg / L ) ] \qquad Formula I$$

In such embodiments, a score of greater than or equal to −1.1903 calculated using Formula I indicates a likelihood of non-survival, and a score of less than −1.1903 calculated using Formula I indicates a likelihood of survival.

In some embodiments, the subject has undergone, is undergoing or is commencing a treatment regimen for treating prostate cancer, such as chemotherapy and/or administration of an androgen receptor signalling inhibitor.

In some embodiments, a subject determined to have a likelihood of non-survival is administered a treatment for prostate cancer; especially wherein the treatment comprises administering a lipid targeted therapy, such as evolocumab or opaganib.

In some embodiments, the levels of the biomarkers are quantitative levels. In specific embodiments, the levels of the biomarkers are determined using mass spectrometry; especially using absolute quantification.

In a further aspect, the invention provides a method for treating or inhibiting the progression of prostate cancer in a subject, comprising, consisting or consisting essentially of:
a) identifying a subject with prostate cancer having a poor prognosis using the method of the invention; and
b) administering a treatment for the prostate cancer.

In some embodiments, the treatment is a lipid targeted therapy, such as evolocumab or opaganib.

Further encompassed, in another aspect, is a composition comprising a biological sample from a subject with prostate cancer and an isotope-labelled lipid corresponding to each of at least three lipid biomarkers in the sample, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0).

In some embodiments, the prostate cancer is castration-resistant prostate cancer; especially a metastatic cancer.

In some embodiments, the lipid biomarkers are selected from the group consisting of ceramide (d18:1/18:0), ceramide (d18:1/22:0), ceramide (d18:1/24:0), ceramide (d18:1/24:1), ceramide (d20:1/24:1) and phosphatidylcholine (16:0/16:0); especially wherein the lipid biomarkers are selected from the group consisting of ceramide (d18:1/18:0), ceramide (d18:1/22:0), ceramide (d18:1/24:0), ceramide (d18:1/24:1) and ceramide (d20:1/24:1). In specific embodiments, the lipid biomarkers are ceramide (d18:1/18:0), ceramide (d18:1/24:0) and ceramide (d18:1/24:1).

In some embodiments, the biological sample is blood, serum or plasma; especially plasma.

In another aspect, there is provided a kit for determining or predicting prognosis for a subject with prostate cancer, comprising one or more reagents for determining the level of at least three lipid biomarkers in a biological sample from the subject, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0).

In specific embodiments, the one or more reagents comprises an isotope-labelled lipid corresponding to each of the at least three lipid biomarkers in the sample.

In some embodiments, the prostate cancer is castration-resistant prostate cancer; especially a metastatic cancer.

In some embodiments, the lipid biomarkers are selected from the group consisting of ceramide (d18:1/18:0), ceramide (d18:1/22:0), ceramide (d18:1/24:0), ceramide (d18:1/24:1), ceramide (d20:1/24:1) and phosphatidylcholine (16:0/16:0); especially wherein the lipid biomarkers are selected from the group consisting of ceramide (d18:1/18:0), ceramide (d18:1/22:0), ceramide (d18:1/24:0), ceramide (d18:1/24:1) and ceramide (d20:1/24:1). In some embodiments, the lipid biomarkers are ceramide (d18:1/18:0), ceramide (d18:1/24:0) and ceramide (d18:1/24:1).

In some embodiments, the biological sample is blood, serum or plasma; especially plasma.

In a still further aspect, there is provided an apparatus for determining an indicator used in assessing prognosis for a subject with prostate cancer, the apparatus comprising at least one electronic processing device that:
a) determines a biomarker level of at least three lipid biomarkers in a biological sample obtained from the subject, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0); and
b) determines the indicator using the derived biomarker levels.

In some embodiments, the prostate cancer is castration-resistant prostate cancer; especially a metastatic cancer.

In some embodiments, the lipid biomarkers are selected from the group consisting of ceramide (d18:1/18:0), ceramide (d18:1/22:0), ceramide (d18:1/24:0), ceramide (d18:1/24:1), ceramide (d20:1/24:1) and phosphatidylcholine (16:0/16:0); especially the group consisting of ceramide (d18:1/18:0), ceramide (d18:1/22:0), ceramide (d18:1/24:0), ceramide (d18:1/24:1) and ceramide (d20:1/24:1). In particular embodiments, the lipid biomarkers are ceramide (d18:1/18:0), ceramide (d18:1/24:0) and ceramide (d18:1/24:1).

In some embodiments, step a) further comprises determining the level of one or more biomarkers selected from the group consisting of total cholesterol, triglycerides and high-density lipoprotein; especially one or more biomarkers selected from the group consisting of total cholesterol and triglycerides. In specific embodiments, step a) further comprises determining the level of total cholesterol and triglycerides.

In particular embodiments, step a) comprises determining the level of ceramide (d18:1/18:0), ceramide (d18:1/24:0), ceramide (d18:1/24:1), total cholesterol and triglycerides.

In some embodiments, the biological sample is blood, serum or plasma; especially plasma.

In another aspect, there is provided a method for selecting a subject with prostate cancer for treatment with a lipid targeted therapy, comprising, consisting or consisting essentially of identifying a subject with prostate cancer having a poor prognosis using the method of the invention.

In particular embodiments, the treatment comprises evolocumab or opaganib.

Further provided is a method for monitoring the response of a subject with prostate cancer to a therapeutic treatment, the method comprising, consisting or consisting essentially of:
a) determining the level of at least three lipid biomarkers in a first biological sample obtained from the subject, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0), and the first biological sample is obtained before or after commencement of treatment;
b) determining a first indicator using the biomarker levels;
c) determining the level of at least three lipid biomarkers in a second biological sample obtained from the subject, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0), and the second biological sample is obtained at a time point after commencement of therapeutic treatment and after the first biological sample is obtained;
d) determining a second indicator using the biomarker levels; and
e) comparing the indicator in the first and second biological samples;
wherein a change in the indicator between the first and second biological samples is indicative of whether or not the subject is responding to the therapeutic treatment.

In another aspect, there is provided a method for monitoring the prognosis for a subject with prostate cancer, the method comprising, consisting or consisting essentially of:
a) determining the level of at least three lipid biomarkers in a first biological sample obtained from the subject, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0);
b) determining a first indicator using the biomarker levels;
c) determining the level of at least three lipid biomarkers in a second biological sample obtained from the subject, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0), and the second biological sample is obtained at a time point after the first biological sample is obtained;
d) determining a second indicator using the biomarker levels; and
e) comparing the indicator in the first and second biological samples;
wherein a change in the indicator between the first and second biological samples is indicative of a change in the prognosis for the subject.

In particular embodiments, the prostate cancer is castration-resistant prostate cancer; especially a metastatic cancer.

In some embodiments, the lipid biomarkers in steps a) and c) are selected from the group consisting of ceramide (d18:1/18:0), ceramide (d18:1/22:0), ceramide (d18:1/24:0), ceramide (d18:1/24:1), ceramide (d20:1/24:1) and phosphatidylcholine (16:0/16:0); especially wherein the lipid biomarkers in steps a) and c) are selected from the group consisting of ceramide (d18:1/18:0), ceramide (d18:1/22:0), ceramide (d18:1/24:0), ceramide (d18:1/24:1) and ceramide (d20:1/24:1). In particular embodiments, the lipid biomarkers in steps a) and c) are ceramide (d18:1/18:0), ceramide (d18:1/24:0) and ceramide (d18:1/24:1).

In particular embodiments, steps a) and c) further comprise determining the level of one or more biomarkers selected from the group consisting of total cholesterol, triglycerides and high-density lipoprotein; especially the level of one or more biomarkers selected from the group consisting of total cholesterol and triglycerides. In specific embodiments, steps a) and c) further comprise determining the level of total cholesterol and triglycerides.

In particular embodiments, steps a) and c) comprise determining the level of ceramide (d18:1/18:0), ceramide (d18:1/24:0), ceramide (d18:1/24:1), total cholesterol and triglycerides.

In particular embodiments, the biological sample is blood, serum or plasma; especially plasma.

### BRIEF DESCRIPTION OF THE DRAWINGS

FIG. 1 is an LC-MS chromatogram for each of the lipid analytes of interest.

FIG. 2 is a series of scatter plots and a scatter plot matrix comparing the targeted assay and high-throughput assay. FIG. 2A is a series of scatter plots of the correlation between individual lipid pairs on the targeted assay and high-throughput assay. FIG. 2B is a scatter plot matrix of pairs of lipid species performed on the targeted assay, utilising Pearson's correlation coefficient. Abbreviations: Cer, Ceramide; R, Pearson's correlation coefficient; and HDL, high density lipoprotein.

FIG. 3 is a series of graphs showing the Kaplan Meier Survival analysis of overall survival by PCPro in (A) the discovery cohort, (B) the validation cohort, (C) those treated with ARSI in the validation cohort, (D) those treated with taxane chemotherapy in the validation cohort, (E) those treated with first line treatment in the validation cohort and (F) those treated with second line treatment in the validation cohort. Abbreviations: ARSI, androgen receptor signalling inhibitor; HR, hazard ratio; mo, months; OS, overall survival; and NR, not reached.

FIG. 4 is a graph showing the correlation of PCPro with the three-lipid prognostic signature in the discovery cohort.

FIG. 5 is a graph showing the correlation of PCPro with the three-lipid prognostic signature in the validation cohort.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much 15, 14, 13, 12, 11, 10, 9, 8, 7, 6, 5, 4, 3, 2 or 1% to a reference quantity, level, value, number, frequency, percentage, dimension, size, amount, weight or length.

The term “agent” includes a compound that induces a desired pharmacological and/or physiological effect. The term also encompasses pharmaceutically acceptable and pharmacologically active ingredients of those compounds specifically mentioned herein including but not limited to salts, esters, amides, prodrugs, active metabolites, analogues and the like. When the above term is used, then it is to be understood that this includes the active agent per se as well as pharmaceutically acceptable, pharmacologically active salts, esters, amides, prodrugs, metabolites, analogues, etc. The term “agent” is not to be construed narrowly but extends to small molecules, proteinaceous molecules such as peptides, polypeptides and proteins as well as compositions comprising them and genetic molecules such as RNA, DNA and mimetics and chemical analogues thereof as well as cellular agents.

As used herein, the term “and/or” refers to and encompasses any and all possible combinations of one or more of the associated listed items, as well as the lack of combinations when interpreted in the alternative (or).

As used herein, the term “biomarker” refers to a naturally occurring biological molecule present in a subject at varying concentrations useful in predicting a prognosis or outcome of a disease or a condition, such as prostate cancer. For example, the biomarker can be a lipid present in higher or lower amounts in a biological sample (e.g. blood, serum or plasma) of a subject with prostate cancer, especially metastatic castration-resistant prostate cancer. Suitable biomarkers include, for example, ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0), sphingomyelin (d18:1/16:0), total cholesterol, triglycerides and high-density lipoprotein.

As used herein, the term “composite score” refers to an aggregation of the obtained levels for biomarkers measured in a sample from a subject, optionally in combination with one or more patient clinical parameters or signs.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. Thus, the use of the term “comprising” and the like indicates that the listed integers are required or mandatory, but that other integers are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified for the listed elements. Thus, the phrase “consisting essentially of” indicates that the listed elements are required or mandatory, but that other elements are optional and may or may not be present depending upon whether or not they affect the activity or action of the listed elements.

As used herein, the term “correlates” or “correlates with” and like terms, refers to a statistical association between two or more things, such as events, characteristics, outcomes, numbers, data sets, etc., which may be referred to as “variables”. It will be understood that the things may be of different types. Often the variables are expressed as numbers (e.g. measurements, values, likelihood or risk), wherein a positive correlation means that as one variable increases, the other also increases, and a negative correlation (also called anti-correlation) means that as one variable increases, the other variable decreases. In various embodiments, correlating a biomarker or biomarker signature with a prognosis (e.g. a favourable outcome or a good prognosis such as no disease progression and/or no death from disease within about 18 months from commencement of chemotherapy or treatment with an androgen receptor signalling inhibitor, or an unfavourable outcome or a poor prognosis such as disease progression and/or death from disease within about 18 months from commencement of chemotherapy or treatment with an androgen receptor signalling inhibitor) comprises determining the level of at least one biomarker in a biological sample from a subject having the specified prognosis or a subject known to be free of that prognosis. In specific embodiments, a profile of biomarker levels is correlated to a global probability or a particular outcome, using receiver operating characteristic (ROC) curves.

The term “cut-off value” as used herein is a level or amount (or concentration) which may be an absolute level or a relative level or amount (or concentration), which is indicative of whether a subject having prostate cancer has a particular prognosis (e.g. a favourable outcome or a good prognosis such as no disease progression and/or no death from disease within about 18 months from commencement of chemotherapy or treatment with an androgen receptor signalling inhibitor, or an unfavourable outcome or a poor prognosis such as disease progression and/or death from disease within about 18 months from commencement of chemotherapy or treatment with an androgen receptor signalling inhibitor). Depending on the biomarker or combination of biomarkers, a subject having prostate cancer is regarded as having a particular prognosis, if either the level of the biomarker(s) detected and determined, respectively, is lower than the cut-off value, or the level of the biomarker(s) detected and determined, respectively, is higher than the cut-off value.

By “effective amount”, in the context of treating or inhibiting the development of a condition is meant the administration of an amount of an agent, compound or composition to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

As used herein, the term “higher” with reference to a biomarker measurement refers to a statistically significant and measurable difference in the level of a biomarker compared to the level of another biomarker or to a control level where the biomarker measurement is greater than the level of the other biomarker or the control level. The difference is suitably at least about 10%, at least about 20%, at least about 30%, at least about 40%, or at least about 50%.

As used herein, the term “increase” or “increased’ with reference to a biomarker level refers to a statistically significant and measurable increase in the biomarker level compared to the level of another biomarker or to a control level. The increase is suitably an increase of at least about 10%, at least about 20%, at least about 30%, at least about 40%, or at least about 50%.

The term “indicator” as used herein refers to a result or representation of a result, including any information, number (e.g. biomarker level including functionalised biomarker level and composite score), ratio, signal, sign, mark, or note by which a skilled person can estimate and/or determine a prognosis for a subject having a given disease or condition, or survival of a subject having a given disease or condition. In the case of the present invention, the “indicator” may optionally be used together with other clinical characteristics, to arrive at a prognosis for a subject with prostate cancer or a determination of survival of a subject with prostate cancer. That such an indicator is “determined” is not meant to imply that the indicator is 100% accurate. The skilled clinician may use the indicator together with other clinical parameters or signs to arrive at a prognosis or determination of survival.

The term “label” is used herein in a broad sense to refer to an agent that is capable of providing a detectable signal, either directly or through interaction with one or more additional members of a signal producing system and that has been artificially added, linked or attached via chemical manipulation to a molecule. Labels can be visual, optical, photonic, electronic, acoustic, optoacoustic, by mass, electro-chemical, electro-optical, spectrometry, enzymatic, or otherwise chemically, biochemically hydrodynamically, electrically or physically detectable. In specific embodiments, a molecule such as a lipid biomarker disclosed herein is labelled with a detectable molecule selected form the group consisting of isotopes, radioisotopes, fluorescent compounds, bioluminescent compounds, chemiluminescent compounds, metal chelators or enzymes. Examples of labels include, but are not limited to, isotopes (e.g. deuterium, 13C, 15N, and the like), radioisotopes (e.g. 3H, 14C, 35S, 1251 and 1311), fluorescent labels (e.g. FITC, rhodamine or lanthanide phosphors), luminescent labels such as luminol, enzymatic labels (e.g. horseradish peroxidase, beta-galactosidase, luciferase, alkaline phosphatase and acetylcholinesterase), biotinyl groups (which can be detected by marked avidin, for example, streptavidin containing a fluorescent marker or enzymatic activity that can be detected by optical or calorimetric methods), predetermined polypeptide epitopes recognised by a secondary reporter (e.g. leucine zipper pair sequences, binding sites for secondary antibodies, metal binding domains and epitope tags).

The “level” or “levels” of a biomarker as used herein is a detectable level of the biomarker in a sample. The level can be measured by methods known to those skilled in the art and also as disclosed herein (e.g. mass spectrometry). These terms encompass a quantitative level (e.g. weight or moles), a semi-quantitative level, a relative level (e.g. weight % or mole % within a class), a concentration and the like. Thus, these terms encompass absolute or relative levels or concentrations of a biomarker in a sample. The levels may be raw or normalised levels or may be mathematically transformed biomarker levels. Alternatively, the biomarker levels could be functionalised biomarker levels, which are levels that have been functionalised from one or more measured biomarker levels, for example by applying a function to the one or more measured biomarker levels. Biomarker levels can be of any appropriate form depending on the manner in which the levels are determined. For example, the biomarker levels could be determined using high-throughput technologies such as mass spectrometry, immunoassays, or any combination of such technologies, especially mass spectrometry.

Lipids are referred to herein using nomenclature standard in the art. For example, particular lipids are referred to herein using the common name for the type of lipid with the structures of the side chains within parentheses in the format: (number of carbon atoms: number of double bond equivalents). Both side chains are defined where appropriate. The inclusion of a “d” designation within the brackets refers to 1,3-dihydroxy. For example, ceramide (d18:1/24:0) is used herein to refer to N-lignoceroyl-D-erythro-sphingosine.

As used herein, the term “lower” with reference to a biomarker measurement refers to a statistically significant and measurable difference in the level of a biomarker compared to the level of another biomarker or to a control level where the biomarker measurement is less than the level of the other biomarker or the control level.

The difference is suitably at least about 10%, at least about 20%, at least about 30%, at least about 40% or at least about 50%.

As used herein, the term “normalisation” and its derivatives, when used in conjunction with measurement of biomarkers across samples and time, refer to mathematical methods, including but not limited to multiple of the median (MoM), standard deviation normalisation, sigmoidal normalisation, etc., where the intention is that these normalised values allow the comparison of corresponding normalised values from different datasets in a way that eliminates or minimises differences and gross influences.

The term “prognosis” as used herein refers to a prediction of the probable course and outcome of a clinical condition or disease. A prognosis is usually made by evaluating factors or symptoms of a disease that are indicative of a favourable or unfavourable course or outcome of the disease. The skilled person will understand that the term “prognosis” refers to an increased probability that a certain course or outcome (e.g. disease progression, no disease progression, death, survival, etc.) will occur; that is, that a course or outcome is more likely to occur in a subject exhibiting a given condition, when compared to those individuals not exhibiting the condition. In some embodiments, prognosis also refers to the ability to demonstrate a positive or negative response to therapy or other treatment regimens, for the disease or condition in the subject. In some embodiments, prognosis refers to the ability to predict the presence or diminishment of disease/condition associated symptoms. A prognostic method may comprise classifying a subject or sample obtained from a subject into one of multiple categories, wherein the categories correlate with different likelihoods that a subject will experience a particular outcome. For example, categories can be low risk and high risk, wherein subjects in the low risk category have a lower likelihood of experiencing a poor outcome (e.g. within a given time period such as 6 months, 1 year, 18 months, 2 years or 3 years) than do subjects in the high risk category. A poor outcome could be, for example, disease progression or death attributable to the disease.

As used herein, the term “reduce” or “reduced” with reference to a biomarker level refers to a statistically significant and measurable reduction in the biomarker level compared to the level of another biomarker or to a control level. The reduction is suitably a reduction of at least about 10%, at least about 20%, at least about 30%, at least about 40%, or at least about 50%.

The term “subject” as used herein refers to a prostate-containing mammalian subject, for whom the assessment of prognosis or survival, or therapy or prophylaxis is desired. Suitable subjects include, but are not limited to, primates; livestock animals such as sheep, cows, horses, deer, donkeys and pigs; laboratory test animals such as rabbits, mice, rats, guinea pigs and hamsters; companion animals such as cats and dogs; and captive wild animals such as foxes, deer and dingoes. In particular, the subject is a human.

As used herein, the terms “treatment”, “treating”, and the like, refer to obtaining a desired pharmacologic and/or physiologic effect. The effect may be therapeutic in terms of a partial or complete cure for a disease, disorder or condition and/or adverse effect attributable to the disease, disorder or condition. These terms also cover any treatment of a condition, disorder or disease in a subject, particularly in a human, and include: (a) inhibiting the disease, disorder or condition, i.e. arresting its development; or (b) relieving the disease, disorder or condition, i.e. causing regression of the disease, disorder or condition. In addition, this term includes palliative treatment, that is, treatment designed for the relief of symptoms rather than the curing of the disease, disorder or condition; preventative treatment, that is, treatment directed to minimising or partially or completely inhibiting the development of the associated disease, disorder or condition; and supportive treatment, that is, treatment employed to supplement another specific therapy directed toward the improvement of the associated disease, disorder or condition. It is understood that treatment, while intended to cure, ameliorate, stabilise, or prevent a disease, pathological condition or disorder, need not actually result in the cure, amelioration, stabilisation or prevention.

As used herein, the term “treatment regimen” refers to prophylactic and/or therapeutic (i.e. after onset of a specified condition) treatments, unless the context specifically indicates otherwise. The term “treatment regimen” encompasses natural substances and pharmaceutical agents (i.e. “drugs”) as well as any other treatment regimen including, but not limited to, dietary treatments, physical therapy or exercise regimens, surgical interventions, radiotherapy, chemotherapy, immunotherapy, lipid targeted therapy and combinations thereof. Desirable effects of treatment include decreasing the rate of disease progression, ameliorating or palliating the disease state, and remission or improved prognosis. For example, an individual is successfully “treated” if one or more symptoms associated with prostate cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of individuals.

Each embodiment described herein is to be applied mutatis mutandis to each and every embodiment unless specifically stated otherwise.

### 2. Abbreviations

The following abbreviations are used throughout the application:
ceramide (d18:1/18:0)=N-stearoyl-D-erythro-sphingosine
ceramide (d18:1/22:0)=N-behenoyl-D-erythro-sphingosine
ceramide (d18:1/24:0)=N-lignoceroyl-D-erythro-sphingosine
ceramide (d18:1/24:1)=N-nervonoyl-D-erythro-sphingosine
ceramide (d20:1/24:0)=N-lignoceroyl-D-erythro-sphingosine (C20 base)
ceramide (d20:1/24:1)=N-nervonoyl-D-erythro-sphingosine (C20 base)
phosphatidylcholine (16:0/16:0)=dipalmitoyl phosphatidylcholine
sphingomyelin (d18:1/16:0)=N-palmitoyl-D-erythro-sphingosylphosphorylcholine
BuMe=1-butanol and methanol mixture
Cer=ceramide
HDL=high-density lipoprotein
HPLC=high performance liquid chromatography
HR=hazard ratio
LASSO=Least Absolute Shrinkage and Selection Operator
LC-MS=liquid chromatography-mass spectrometry
NR=not reached
OS=overall survival
PC=phosphatidylcholine
R=Pearson's Correlation Coefficient
R2=coefficient of determination
ROC=Receiver Operating Characteristic
SM=sphingomyelin

### 3. Biomarkers for Determining Prognosis for Subjects with Prostate Cancer

The present invention is based on the discovery that abnormal lipid levels can be used to identify subjects with prostate cancer that are likely to be resistant to conventional treatment, for example, with chemotherapy of treatment with an androgen receptor signalling inhibitor and, accordingly, have a poor prognosis and reduced overall survival. The inventors have conceived that levels of specific lipids (e.g. ceramides, phosphatidylcholine and/or sphingomyelin), optionally together with other markers such as total cholesterol, triglycerides and high-density lipoprotein, may be used in kits, compositions and methods for determining or predicting prognosis for and survival of a subject with prostate cancer, particularly a metastatic castration-resistant prostate cancer.

In one aspect, there is provided a method for determining an indicator used in determining prognosis for a subject with prostate cancer, the method comprising, consisting or consisting essentially of:
a) determining the level of at least three lipid biomarkers in a biological sample obtained from the subject, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0); and
b) determining the indicator using the biomarker levels.

While the prognosis for subjects with all types of prostate cancer may be determined using the methods, the methods are particularly useful for determining the prognosis for a subject with castration-resistant prostate cancer. The prostate cancer may be a metastatic cancer, such as metastatic castration-resistant prostate cancer. In some embodiments, the subject has undergone, is undergoing or is commencing a treatment regimen for treating prostate cancer, such as chemotherapy (e.g. docetaxel, cabazitaxel, mitoxantrone, estramustine or carboplatin), administration of an androgen receptor signalling inhibitor (e.g. abiraterone, enzalutamide, apalutamide or daralutamide), administration of a targeted radioisotope (e.g. Lutetium-177 Prostate Specific Membrane Antigen) and/or administration of a poly-ADP-ribose polymerase (PARP) inhibitor (e.g. olaparib, rucaparib or niraparib).

The lipid biomarkers, in some embodiments, are selected from the group consisting of ceramide (d18:1/18:0), ceramide (d18:1/22:0), ceramide (d18:1/24:0), ceramide (d18:1/24:1), ceramide (d20:1/24:1) and phosphatidylcholine (16:0/16:0). In particular embodiments, the lipid biomarkers are selected from the group consisting of ceramide (d18:1/18:0), ceramide (d18:1/22:0), ceramide (d18:1/24:0), ceramide (d18:1/24:1) and ceramide (d20:1/24:1). In specific embodiments, the lipid biomarkers are ceramide (d18:1/18:0), ceramide (d18:1/24:0) and ceramide (d18:1/24:1).

While the method may involve determining the levels of the above listed lipids only, in some embodiments, the method further comprises determining the level of total cholesterol (i.e. the total level of cholesterol in the sample, including low-density lipoprotein cholesterol and high-density lipoprotein cholesterol), triglycerides (i.e. the total level of triglycerides in the sample) and/or high-density lipoprotein. Accordingly, in some embodiments, step a) further comprises determining the level of one or more biomarkers selected from the group consisting of total cholesterol, triglycerides and high-density lipoprotein; especially one or more biomarkers selected from the group consisting of total cholesterol and triglycerides. In specific embodiments, step a) further comprises determining the level of total cholesterol and triglycerides. In particular embodiments, step a) comprises determining the level of ceramide (d18:1/18:0), ceramide (d18:1/24:0), ceramide (d18:1/24:1), total cholesterol and triglycerides.

In an alternative aspect, step a) comprises determining the level of at least three biomarkers in a biological sample obtained from the subject, wherein the biomarkers comprise at least one biomarker selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0); and at least two biomarkers selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0), sphingomyelin (d18:1/16:0), total cholesterol, triglycerides and high-density lipoprotein. In particular embodiments, step a) comprises determining the level of ceramide (d18:1/18:0), ceramide (d18:1/24:0), ceramide (d18:1/24:1), total cholesterol and triglycerides.

In some embodiments, the method comprises determining the level of ceramide (d18:1/18:0), ceramide (d18:1/24:0), total cholesterol and triglycerides; ceramide (d18:1/18:0), ceramide (d18:1/24:0) and ceramide (d20:1/24:1); ceramide (d18:1/18:0), ceramide (d18:1/24:1), ceramide (d18:1/22:0), total cholesterol and triglycerides; ceramide (d18:1/18:0), ceramide (d18:1/22:0), ceramide (d18:1/24:0), ceramide (d18:1/24:1), ceramide (d20:1/24:0), ceramide (d20:1/24:1) and total cholesterol; ceramide (d18:1/18:0), ceramide (d18:1/22:0), ceramide (d18:1/24:0), ceramide (d18:1/24:1), ceramide (d20:1/24:0) and ceramide (d20:1/24:1); ceramide (d18:1/18:0), ceramide (d18:1/24:1), phosphatidylcholine (16:0/16:0) and total cholesterol; ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and total cholesterol; or ceramide (d18:1/18:0), ceramide (d18:1/24:0), ceramide (d18:1/24:1) and total cholesterol.

In some embodiments, the method further comprises applying a function to biomarker level(s) to yield at least one functionalised biomarker level and determining the indicator using the at least one functionalised biomarker level. For example, the function may include at least one of multiplying, dividing, adding and subtracting biomarker levels.

The method may further comprise combining the biomarker levels and/or functionalised biomarker levels to provide a composite score and determining the indicator using the composite score. In particular embodiments, the biomarker levels and/or functionalised biomarker levels are combined by adding, multiplying, subtracting, and/or dividing biomarker levels and/or functionalised biomarker levels.

The biomarker levels, functionalised biomarker level(s) or composite score may be further analysed with reference to a corresponding reference biomarker level range or cut-off levels, functionalised biomarker level range or cut-off levels, or reference composite score range or cut-off scores, to determine the indicator. For example, the reference level, score or range or cut-off levels or scores thereof may be obtained from a control biological sample obtained from a reference population of subjects with prostate cancer having a favourable outcome (e.g. no disease progression and/or no death from disease within about 18 months from commencement of chemotherapy or treatment with an androgen receptor signalling inhibitor) or an unfavourable outcome (e.g. disease progression and/or death from disease within about 18 months from commencement of chemotherapy or treatment with an androgen receptor signalling inhibitor); especially a favourable outcome.

The methods of the invention may be useful for determining whether the subject has a likelihood of a poor prognosis or a likelihood of a good prognosis. For example, a poor prognosis may comprise an unfavourable outcome, such as disease progression and/or death from disease within about 18 months from commencement of chemotherapy or treatment with an androgen receptor signalling inhibitor. A good prognosis, conversely, may comprise a favourable outcome, such as no disease progression and/or no death from disease within about 18 months from commencement of chemotherapy or treatment with an androgen receptor signalling inhibitor.

Suitably, the indicator indicates a likelihood of a poor prognosis, if the biomarker level(s), functionalised biomarker level(s) or composite score is (are) indicative of the level of the biomarker(s) in the sample that correlates with an increased likelihood of a poor prognosis relative to a predetermined reference biomarker level range or cut-off level, predetermined reference functionalized biomarker level range or cut-off level, or predetermined reference composite score range or cut-off value.

Alternatively, the indicator indicates a likelihood of a good prognosis, if the biomarker level(s), functionalised biomarker level(s) or composite score is (are) indicative of the level of the biomarker(s) in the sample that correlates with an increased likelihood of a good prognosis relative to a predetermined reference biomarker level range or cut-off level, predetermined reference functionalised biomarker level range or cut-off level, or predetermined reference composite score range or cut-off value.

In specific embodiments, the indicator indicates a likelihood of a poor prognosis if: the level of ceramide (d18:1/18:0) is higher than in control biological samples obtained from a reference population of subjects with prostate cancer with a favourable outcome; the level of ceramide (d18:1/22:0) is lower than in control biological samples obtained from a reference population of subjects with prostate cancer with a favourable outcome; the level of ceramide (d18:1/24:0) is lower than in control biological samples obtained from a reference population of subjects with prostate cancer with a favourable outcome; the level of ceramide (d18:1/24:1) is higher than in control biological samples obtained from a reference population of subjects with prostate cancer with a favourable outcome; the level of ceramide (d20:1/24:0) is lower than in control biological samples obtained from a reference population of subjects with prostate cancer with a favourable outcome; the level of ceramide (d20:1/24:1) is higher than in control biological samples obtained from a reference population of subjects with prostate cancer with a favourable outcome; and/or the level of phosphatidylcholine (16:0/16:0) is higher than in control biological samples obtained from a reference population of subjects with prostate cancer with a favourable outcome. Additionally, when step a) further comprises determining the level of one or more biomarkers selected from the group consisting of total cholesterol, triglycerides and high-density lipoprotein, the indicator indicates a likelihood of a poor prognosis if: the level of total cholesterol is lower than in control biological samples obtained from a reference population of subjects with prostate cancer with a favourable outcome; the level of triglycerides is lower than in control biological samples obtained from a reference population of subjects with prostate cancer with a favourable outcome; and/or the level of high-density lipoprotein is lower than in control biological samples obtained from a reference population of subjects with prostate cancer with a favourable outcome.

In particular embodiments, when step a) further comprises determining the level of one or more biomarkers selected from the group consisting of total cholesterol, triglycerides and high-density lipoprotein, the indicator indicates a likelihood of a poor prognosis if: the level of ceramide (d18:1/18:0) is higher than in control biological samples obtained from a reference population of subjects with prostate cancer with a favourable outcome; the level of ceramide (d18:1/22:0) is lower than in control biological samples obtained from a reference population of subjects with prostate cancer with a favourable outcome; the level of ceramide (d18:1/24:0) is lower than in control biological samples obtained from a reference population of subjects with prostate cancer with a favourable outcome; the level of ceramide (d18:1/24:1) is higher than in control biological samples obtained from a reference population of subjects with prostate cancer with a favourable outcome; the level of ceramide (d20:1/24:0) is lower than in control biological samples obtained from a reference population of subjects with prostate cancer with a favourable outcome; the level of ceramide (d20:1/24:1) is higher than in control biological samples obtained from a reference population of subjects with prostate cancer with a favourable outcome; the level of phosphatidylcholine (16:0/16:0) is higher than in control biological samples obtained from a reference population of subjects with prostate cancer with a favourable outcome; the level of total cholesterol is lower than in control biological samples obtained from a reference population of subjects with prostate cancer with a favourable outcome; the level of triglycerides is lower than in control biological samples obtained from a reference population of subjects with prostate cancer with a favourable outcome; and/or the level of high-density lipoprotein is lower than in control biological samples obtained from a reference population of subjects with prostate cancer with a favourable outcome.

In specific embodiments, the method comprises determining the indicator by dividing the level of ceramide (d18:1/24:0) by the level of ceramide (d18:1/24:1), and adding this value to the level of ceramide (d18:1/18:0), the level of total cholesterol, and the level of triglycerides, or functionalised levels of any of the foregoing levels.

For example, in some embodiments, the method comprises determining the indicator using Formula II:

$$[ A \times the level of ceramide ( d 18 : 1 / 18 : 0 ) in mg / L ] + [ B \times the level of total cholesterol in mmol / L ] + [ C \times the level of triglycerides in mmol / L ] + [ D \times ( the level of ceramide ( d 18 : 1 / 24 : 0 ) in mg / L / the level of ceramide ( d 18 : 1 / 24 : 1 ) in mg / L ) ] \qquad Formula II$$

wherein:
A is a number in the range of from about 9 to about 11 (and all one ten thousandth integers therebetween);
B is a number in the range of from about −0.1 to about −0.4 (and all one ten thousandth integers therebetween);
C is a number in the range of from about −0.1 to about −0.4 (and all one ten thousandth integers therebetween); and
D is a number in the range of from about −0.1 to about −0.4 (and all one ten thousandth integers therebetween).

In some embodiments, A is a number in the range of from about 9.5 to about 10.5; especially about 10; more especially about 10.0506.

In suitable embodiments, B is a number in the range of from about −0.15 to about −0.35; especially in the range of from about −0.2 to about −0.3; more especially about −0.28; most especially about −0.2783.

In some embodiments, C is a number in the range of from about −0.15 to about −0.35; especially in the range of from about −0.2 to about −0.3; more especially about −0.22; most especially about −0.2240.

In particular embodiments, D is a number in the range of from about −0.15 to about −0.35; especially in the range of from about −0.25 to about −0.35; more especially about −0.3; most especially about −0.2979.

In particular embodiments, the method comprises determining the indicator using Formula I:

$$[ 10.0506 \times the level of ceramide ( d 18 : 1 / 18 : 0 ) in mg / L ] + [ - 0.2783 \times the level of total cholesterol in mmol / L ] + [ - 0.224 \times the level of triglycerides in mmol / L ] + [ - 0.2979 \times ( the level of ceramide ( d 18 : 1 / 24 : 0 ) in mg / L / the level of ceramide ( d 18 : 1 / 24 : 1 ) in mg / L ) ] \qquad Formula I$$

A likelihood of a poor prognosis may be indicated when a score of greater than or equal to about −0.5 to about −2 (and all one ten thousandth integers therebetween) is calculated using Formula I or III; especially a score of greater than or equal to about −1.3 to about −1.16; more especially a score of equal to or greater than about −1.1903. Scores of less than this value indicate a likelihood of a good prognosis, especially a score of less than about −1.1903.

In particular embodiments, a score of greater than or equal to about −1.1903 calculated using Formula I indicates a likelihood of a poor prognosis, and a score of less than about −1.1903 calculated using Formula I indicates a likelihood of a good prognosis.

In alternative embodiments, the method comprises determining the indicator by subtracting the level of ceramide (d18:1/24:1) from the level of ceramide (d18:1/24:0), and adding this value to the level of ceramide (d18:1/18:0), the level of total cholesterol, and the level of triglycerides, or functionalised levels of any of the foregoing levels. In some embodiments, the method comprises determining the indicator using Formula III:

$$[ E \times the level of ceramide ( d 18 : 1 / 18 : 0 ) in mg / L ] + [ F \times the level of total cholesterol in mmol / L ] + [ G \times the level of triglycerides in mmol / L ] + \{ H \times [ the level of ceramide ( d 18 : 1 / 24 : 0 ) in mg / L ] - [ the level of ceramide ( d 18 : 1 / 24 : 1 ) in mg / L ] \} \qquad Formula III$$

wherein:
E is a number in the range of from about 10 to about 12 (and all one ten thousandth integers therebetween);
F is a number in the range of from about −0.1 to about −0.4 (and all one ten thousandth integers therebetween);
G is a number in the range of from about −0.1 to about −0.4 (and all one ten thousandth integers therebetween); and
H is a number in the range of from about −0.1 to about −0.4 (and all one ten thousandth integers therebetween).

In some embodiments, E is a number in the range of from about 10.5 to about 11.5; especially about 11; more especially about 10.9903.

In suitable embodiments, F is a number in the range of from about −0.15 to about −0.35; especially in the range of from about −0.2 to about −0.3; more especially about −0.28; most especially about −0.2779.

In some embodiments, G is a number in the range of from about −0.15 to about −0.35; especially in the range of from about −0.2 to about −0.3; more especially about −0.22; most especially about −0.2243.

In particular embodiments, H is a number in the range of from about −0.15 to about −0.35; especially in the range of from about −0.2 to about −0.3; more especially about −0.24; most especially about −0.2366.

In particular embodiments, the method comprises determining the indicator using Formula IV:

$$[ 10.9903 \times the level of ceramide ( d 18 : 1 / 18 : 0 ) in mg / L ] + [ - 0.2779 \times the level of total cholesterol in mmol / L ] + [ - 0.2243 \times the level of triglycerides in mmol / L ] + \{ - 0.2366 \times [ the level of ceramide ( d 18 : 1 / 24 : 0 ) in mg / L ] - [ the level of ceramide ( d 18 : 1 / 24 : 1 ) in mg / L ] \} \qquad Formula IV .$$

A likelihood of a poor prognosis may be indicated when a score of greater than or equal to about −0.5 to about −1.5 (and all one thousandth integers therebetween) is calculated using Formula III or IV; especially a score of greater than or equal to about −0.9 to about −0.75; more especially a score of equal to or greater than about −0.817. Scores of less than this value indicate a likelihood of a good prognosis, especially a score of less than about −0.817.

In particular embodiments, a score of greater than or equal to about −0.817 calculated using Formula IV indicates a likelihood of a poor prognosis, and a score of less than about −0.817 calculated using Formula IV indicates a likelihood of a good prognosis.

A subject determined to have a likelihood of a poor prognosis may be administered a treatment for prostate cancer. The subject may already be undergoing or commencing a treatment regimen for treating prostate cancer, such as chemotherapy (e.g. docetaxel, cabazitaxel, mitoxantrone, estramustine or carboplatin), administration of an androgen receptor signalling inhibitor (e.g. abiraterone or enzalutamide), and/or administration of a poly-ADP-ribose polymerase (PARP) inhibitor (e.g. olaparib, rucaparib or niraparib). In such embodiments, the subject may be administered a further treatment for prostate cancer or an alternative treatment for the cancer. The treatment may, for example, be more aggressive than the standard treatments for the cancer (e.g. chemotherapy or administration of an androgen receptor signalling inhibitor and/or a PARP inhibitor). In particular embodiments, the treatment is a lipid targeted therapy (i.e. a therapy directly or indirectly targeting one or more lipids, such as lipid activity, lipid metabolism or lipid synthesis), such as a ceramide inhibitor. Suitable lipid targeted therapies include, but are not limited to, a statin (e.g. atorvastatin, fluvastatin, lovastatin, mevastatin, pitavastatin, pravastatin, rosuvastatin or simvastatin), a proprotein convertase subtilisin/kexin type 9 (PCSK9) inhibitor (e.g. evolocumab or alirocumab) a sphingosine kinase 1 and/or 2 inhibitor (e.g. opaganib, PF-543 (1-[[4-[3-methyl-5-[(phenylsulfonyl)methyl]phenoxy]methyl]phenyl]methyl]-2R-pyrrolidinemethanol), SKI-349 (4-amino-2-((4-methoxyphenyl)amino) thiazol-5-yl) (3,4-dimethoxyphenyl) methanone), BML-258 (SKI-I; N′-[(2-hydroxy-1-naphthyl)methylene]-3-(2-naphthyl)-1H-pyrazole-5-carbohydrazide), LCL351 (L-erythro-2-N-(1′-carboxamidine)-sphingosine hydrochloride), LCL146 (D-erythro-2-N-(1′-carboxamidine)-sphingosine hydrochloride), VPC96091 ((S)-1-(4-dodecylbenzoyl) pyrrolidine-2-carboximidamide hydrochloride), SLP7111228 ((S)-2-((3-(4-octylphenyl)-1,2,4-oxadiazol-5-yl)methyl) pyrrolidine-1-carboximidamide hydrochloride), RB-005 (1-(4-octylphenethyl) piperidin-4-amine), (2S,3S)—N—((S)-1-(4-(5-(2-cyclopentylethyl)-1,2,4-oxadiazol-3-yl)phenyl)ethyl)-3-hydroxypyrrolidine-2-carboxamide, SKI-II (2-(p-hydroxyanilino)-4-(p-chlorophenyl) thiazole), MP-A08 (4-methyl-N-[2-[[2-[(4-methylphenyl) sulfonylamino]phenyl] iminomethyl]phenyl]benzenesulfonamide), safingol ((2S,3S)-2-aminooctadecane-1,3-diol), balanocarpol, SLR080811 ((S)-2-[3-(4-octylphenyl)-1,2,4-oxadiazol-5-yl]pyrrolidine-1-carboximidamide), Trans-12a [(1r,4r)-N, N, N-trimethyl-4 (4-octylphenyl)cyclohexanaminium iodide], K145 (3-(2-amino-ethyl)-5-[3-(4-butoxyl-phenyl)-propylidene]-thiazolidine-2,4-dione), (R)-FTY720-OMe [(2R)-2-amino-3-(O-methyl)-(2-(4′-n-octylphenyl)ethyl) propanol], SG-12 [(2S,3R)-2-amino-4-(4-octylphenyl) butane-1,3-diol], and the like), a dihydroceramide desaturase (Des1) inhibitor (e.g. XM462 (N-((2S,3S)-1,3-dihydroxy-4-(tridecylthio) butan-2-yl) octanamide), SKI-II, fenretinide, resveratrol and the like) and/or a sterol regulatory element-binding protein (SREBP) inhibitor (e.g. fatostatin and betulin). In particular embodiments, the subject is administered a PCSK9 inhibitor, such as evolocumab, or a sphingosine kinase 1 and/or 2 inhibitor, such as opaganib.

The biological sample from the subject may be any sample that contains one or more of the lipid biomarkers. For example, in some embodiments, the biological sample is blood, serum or plasma; especially plasma. A suitable biological sample may be obtained by any means known in the art, such as drawing of blood from a subject into a collection tube using a syringe. This may be followed by separation of the serum or plasma from the remaining blood components using suitable means, for example, by clotting and/or centrifugation.

The biological sample may then be enriched for the lipid components, or the lipid components may be extracted from the samples. For example, the sample may be mixed with a suitable organic solvent, such as an alcohol (e.g. 1-butanol, methanol, ethanol, isopropanol and the like, and combinations thereof), which may optionally comprise a protein precipitant such as ammonium formate or ammonium sulfate, followed by sonication and centrifugation. A skilled person will be well aware of suitable methods for lipid extraction and/or enrichment of samples.

The level of the biomarkers may be measured or assessed using any appropriate technique or means known to those of skill in the art. For example, the level of the biomarkers may be assessed using an antibody-based technique, non-limiting examples of which include immunoassays, such as the enzyme-linked immunosorbent assay (ELISA) and the radioimmunoassay (RIA) or mass spectrometry (MS) methods, including Liquid Chromatography-Mass Spectrometry (LC-MS), Direct Analysis in Real Time Mass Spectrometry (DART MS), SELDI-TOF and MALDI-TOF, gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g. MS/MS, MS/MS/MS, ESI-MS/MS, etc.). In particular embodiments, the mass spectrometry may include multiple-reaction monitoring. The level of particular biomarkers, such as cholesterol, triglycerides and high-density lipoprotein, may, alternatively, be determined using enzymatic techniques, such as enzymatic reaction. For example, total cholesterol may be measured by converting esterified cholesterol to cholesterol using cholesterol esterase, which is then acted upon by cholesterol oxidase to produce cholest-4-en-3-one and hydrogen peroxide. The hydrogen peroxide is then reacted with 4-aminophenazone in the presence of peroxidase to produce a coloured product (e.g. a red quinone-imine dye) that is measured at 505 nm (e.g. as described in Barbosa et al. (2019) Molecules, 24 (16): 2890). The total cholesterol may be measured using commercially available assays, such as CHOL2, Cholesterol Gen.2 (Reference No. 05168538 190* or 05168538 214*; Roche Diagnostics, F. Hoffmann-La Roche AG, Basel, Switzerland), which is to be used with a COBAS analyser. The level of triglycerides may be determined using, for example, reaction with lipase, and detection of the glycerol produced by the reaction. For example, the amount of triglycerides may be measured by converting triglycerides to glycerol using lipoprotein lipase, followed by oxidation to dihydroxyacetone phosphate and hydrogen peroxide. The hydrogen peroxide is then reacted with 4-aminophenazone and 4-chlorophenol under the catalytic action of peroxidase to form a red dyestuff. The intensity of the colour of the red dyestuff formed can be measured photometrically and is directly proportional to the triglyceride concentration. A suitable commercially available assay is, for example, TRIGL, triglycerides (Reference No. 05171407 190* or 05171407 214*; Roche Diagnostics, F. Hoffmann-La Roche AG, Basel, Switzerland), which is to be used with a COBAS analyser. The assessment of the level of high-density lipoprotein may be performed using, for example, PEG-cholesterol esterase and PEG-cholesterol oxidase or the protocol described in Moshides (1988) Scandinavian Journal of Clinical and Laboratory Investigation, 48 (1): 59-64. For example, non-HDL lipoproteins are combined with polyanions and a detergent forming a water soluble complex, which blocks the enzymatic reaction of non-HDL lipoproteins. HDL-cholesterol esters are broken down into free HDL-cholesterol and fatty acids by cholesterol esterase. In the presence of oxygen, HDL-cholesterol is oxidised by cholesterol oxidase to Δ⁴-cholestenone and hydrogen peroxide. In the presence of peroxidase, the hydrogen peroxide then reacts with 4-amino-antipyrine and N-ethyl-N-(3-methylphenyl)-N′-succinylethylenediamine to form a dye. The colour intensity of the dye can be measured photometrically and is directly proportional to the HDL-cholesterol concentration. A suitable commercially available assay is, for example, HDLC4, HDL-Cholesterol Gen.4 (Reference No. 07528582 190* or 07528582 214*; Roche Diagnostics, F. Hoffmann-La Roche AG, Basel, Switzerland), which is to be used with a COBAS analyser. Such reactions may be carried out in a high throughput manner, such as by using an analyser (e.g. a COBAS 8000 Analyser; Roche Diagnostics, F. Hoffmann-La Roche AG, Basel, Switzerland). In particular embodiments, the levels of the biomarkers are quantitative levels.

In specific embodiments, the levels of the biomarkers are determined using mass spectrometry, preferably using absolute quantification. For example, the sample may include a labelled internal standard for each biomarker of interest, such a stable isotopically labelled internal standard, representative examples of which include D₇-ceramide (d18:1/18:0), D₇-ceramide (d18:1/22:0), D₇-ceramide (d18:1/24:0), D₇-ceramide (d18:1/24:1), D₇-ceramide (d20:1/24:0), D₇-ceramide (d20:1/24:1), D₉-phosphatidylcholine (16:0/16:0), D₉-sphingomyelin (d18:1/16:0) or D₃₁-sphingomyelin (d18:1/16:0); especially D₇-ceramide (d18:1/18:0), D₇-ceramide (d18:1/24:0), D₇-ceramide (d18:1/24:1), D₉-phosphatidylcholine (16:0/16:0), D₉-sphingomyelin (d18:1/16:0) or D₃₁-sphingomyelin (d18:1/16:0).

In some embodiments, the levels of one or more of the ceramides, phosphatidylcholine and/or sphingomyelin are determined using mass spectrometry and the levels of the total cholesterol, triglycerides and/or high-density lipoprotein are determined using enzymatic reaction.

In another aspect, there is provided a method for determining or predicting prognosis for a subject with prostate cancer, the method comprising, consisting or consisting essentially of:
a) determining the level of at least three lipid biomarkers in a biological sample obtained from the subject, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0);
b) comparing the level of the biomarkers to the respective levels of the corresponding biomarkers in a reference sample; and
c) determining or predicting prognosis for the subject based on the determined levels of the biomarkers and said comparison.

Suitable embodiments of the method, including suitable biological samples and exemplary combinations of biomarkers, are as discussed above.

In some embodiments, the reference sample is a control biological sample obtained from a reference population of subjects with prostate cancer having a favourable outcome (e.g. no disease progression and/or no death from disease within about 18 months from commencement of chemotherapy or treatment with an androgen receptor signalling inhibitor).

In particular embodiments, predicting prognosis comprises predicting subject survival, such as survival beyond about 18 months from commencement of chemotherapy or treatment with an androgen receptor signalling inhibitor.

In some embodiments, the prostate cancer is castration-resistant prostate cancer. In particular embodiments, the prostate cancer is a metastatic cancer, especially metastatic castration-resistant prostate cancer.

In some embodiments, the lipid biomarkers are selected from the group consisting of ceramide (d18:1/18:0), ceramide (d18:1/22:0), ceramide (d18:1/24:0), ceramide (d18:1/24:1), ceramide (d20:1/24:1) and phosphatidylcholine (16:0/16:0); especially wherein the lipid biomarkers are selected from the group consisting of ceramide (d18:1/18:0), ceramide (d18:1/22:0), ceramide (d18:1/24:0), ceramide (d18:1/24:1) and ceramide (d20:1/24:1). In specific embodiments, the lipid biomarkers are ceramide (d18:1/18:0), ceramide (d18:1/24:0) and ceramide (d18:1/24:1).

In some embodiments, the method further comprises determining the level of total cholesterol, triglycerides and/or high-density lipoprotein. In such embodiments, step a) further comprises determining the level of one or more biomarkers selected from the group consisting of total cholesterol, triglycerides and high-density lipoprotein; especially wherein step a) further comprises determining the level of one or more biomarkers selected from the group consisting of total cholesterol and triglycerides. In specific embodiments, step a) further comprises determining the level of total cholesterol and triglycerides. In specific embodiments, step a) comprises determining the level of ceramide (d18:1/18:0), ceramide (d18:1/24:0), ceramide (d18:1/24:1), total cholesterol and triglycerides.

Alternatively, step a) may comprise determining the level of at least three biomarkers in a biological sample obtained from the subject, wherein the biomarkers comprise at least one biomarker selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0); and at least two biomarkers selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0), sphingomyelin (d18:1/16:0), total cholesterol, triglycerides and high-density lipoprotein. In particular embodiments, step a) comprises determining the level of ceramide (d18:1/18:0), ceramide (d18:1/24:0), ceramide (d18:1/24:1), total cholesterol and triglycerides.

In some embodiments, the method comprises determining the level of ceramide (d18:1/18:0), ceramide (d18:1/24:0), total cholesterol and triglycerides; ceramide (d18:1/18:0), ceramide (d18:1/24:0) and ceramide (d20:1/24:1); ceramide (d18:1/18:0), ceramide (d18:1/24:1), ceramide (d18:1/22:0), total cholesterol and triglycerides; ceramide (d18:1/18:0), ceramide (d18:1/22:0), ceramide (d18:1/24:0), ceramide (d18:1/24:1), ceramide (d20:1/24:0), ceramide (d20:1/24:1) and total cholesterol; ceramide (d18:1/18:0), ceramide (d18:1/22:0), ceramide (d18:1/24:0), ceramide (d18:1/24:1), ceramide (d20:1/24:0) and ceramide (d20:1/24:1); ceramide (d18:1/18:0), ceramide (d18:1/24:1), phosphatidylcholine (16:0/16:0) and total cholesterol; ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and total cholesterol; or ceramide (d18:1/18:0), ceramide (d18:1/24:0), ceramide (d18:1/24:1) and total cholesterol.

Suitable biological samples are discussed supra. In some embodiments, the biological sample is blood, serum or plasma; especially plasma.

The methods of the invention may be useful for determining whether the subject has a likelihood of a poor prognosis or a likelihood of a good prognosis. For example, a poor prognosis may comprise an unfavourable outcome, such as disease progression and/or death from disease within about 18 months from commencement of chemotherapy or treatment with an androgen receptor signalling inhibitor. A good prognosis, conversely, may comprise a favourable outcome, such as no disease progression and/or no death from disease within about 18 months from commencement of chemotherapy or treatment with an androgen receptor signalling inhibitor.

Where the reference sample is a control biological sample obtained from a reference population of subjects with prostate cancer having a favourable outcome, the subject may have a likelihood of a poor prognosis if: the level of ceramide (d18:1/18:0) is higher than in the reference sample; the level of ceramide (d18:1/22:0) is lower than in the reference sample; the level of ceramide (d18:1/24:0) is lower than in the reference sample; the level of ceramide (d18:1/24:1) is higher than in the reference sample; the level of ceramide (d20:1/24:0) is lower than in the reference sample; the level of ceramide (d20:1/24:1) is higher than in the reference sample; and/or the level of phosphatidylcholine (16:0/16:0) is higher than in the reference sample. When step a) further comprises determining the level of one or more biomarkers selected from the group consisting of total cholesterol, triglycerides and high-density lipoprotein, the subject may have a likelihood of a poor prognosis if: the level of total cholesterol is lower than in the reference sample; the level of triglycerides is lower than in the reference sample; and/or the level of high-density lipoprotein is lower than in the reference sample. In particular embodiments, the subject may have a likelihood of a poor prognosis if: the level of ceramide (d18:1/18:0) is higher than in the reference sample; the level of ceramide (d18:1/22:0) is lower than in the reference sample; the level of ceramide (d18:1/24:0) is lower than in the reference sample; the level of ceramide (d18:1/24:1) is higher than in the reference sample; the level of ceramide (d20:1/24:0) is lower than in the reference sample; the level of ceramide (d20:1/24:1) is higher than in the reference sample; the level of phosphatidylcholine (16:0/16:0) is higher than in the reference sample; the level of total cholesterol is lower than in the reference sample; the level of triglycerides is lower than in the reference sample; and/or the level of high-density lipoprotein is lower than in the reference sample.

The method may comprise applying a function to biomarker levels, and/or combining the biomarker levels and/or functionalised biomarker levels to provide a composite score as discussed above, which may then be compared to the levels or score of the reference sample. For example, in some embodiments, the method comprises determining the prognosis for the subject by dividing the level of ceramide (d18:1/24:0) by the level of ceramide (d18:1/24:1), and adding this value to the level of ceramide (d18:1/18:0), the level of total cholesterol, and the level of triglycerides, or functionalised levels of any of the foregoing levels. In such embodiments, the level of ceramides may suitably be in mg/L, and the level of total cholesterol and triglycerides may be in mmol/L. This may then be compared to the score obtained for a reference sample, such as a score for a population of subjects having prostate cancer with a favourable outcome (e.g. no disease progression and/or no death from disease within about 18 months from commencement of chemotherapy or treatment with an androgen receptor signalling inhibitor).

For example, in some embodiments, the method comprises determining the indicator using Formula II:

$$[ A \times the level of ceramide ( d 18 : 1 / 18 : 0 ) in mg / L ] + [ B \times the level of total cholesterol in mmol / L ] + [ C \times the level of triglycerides in mmol / L ] + [ D \times ( the level of ceramide ( d 18 : 1 / 24 : 0 ) in mg / L / the level of ceramide ( d 18 : 1 / 24 : 1 ) in mg / L ) ] \qquad Formula II$$

wherein:
A is a number in the range of from about 9 to about 11 (and all one ten thousandth integers therebetween);
B is a number in the range of from about −0.1 to about −0.4 (and all one ten thousandth integers therebetween);
C is a number in the range of from about −0.1 to about −0.4 (and all one ten thousandth integers therebetween); and
D is a number in the range of from about −0.1 to about −0.4 (and all one ten thousandth integers therebetween).

Suitable embodiments of A, B, C and D are discussed supra.

In specific embodiments, the method comprises determining the prognosis for the subject using Formula I:

$$[ 10.0506 \times the level of ceramide ( d 18 : 1 / 18 : 0 ) in mg / L ] + [ - 0.2783 \times the level of total cholesterol in mmol / L ] + [ - 0.224 \times the level of triglycerides in mmol / L ] + [ - 0.2979 \times ( the level of ceramide ( d 18 : 1 / 24 : 0 ) in mg / L / the level of ceramide ( d 18 : 1 / 24 : 1 ) in mg / L ) ] \qquad Formula I .$$

A likelihood of a poor prognosis may be indicated when a score of greater than or equal to about −0.5 to about −2 (and all one ten thousandth integers therebetween) is calculated using Formula I or II; especially a score of greater than or equal to about −1.3 to about −1.16; more especially a score of equal to or greater than about −1.1903. Scores of less than this value indicate a likelihood of a good prognosis, especially a score of less than about −1.1903.

In particular embodiments, a score of greater than or equal to about −1.1903 calculated using Formula I indicates a likelihood of a poor prognosis, and a score of less than about −1.1903 calculated using Formula I indicates a likelihood of a good prognosis.

In alternative embodiments, the method comprises determining the prognosis for the subject by subtracting the level of ceramide (d18:1/24:1) from the level of ceramide (d18:1/24:0), and adding this value to the level of ceramide (d18:1/18:0), the level of total cholesterol, and the level of triglycerides, or functionalised levels of any of the foregoing levels. In some embodiments, the method comprises determining the indicator using Formula III:

$$[ E \times the level of ceramide ( d 18 : 1 / 18 : 0 ) in mg / L ] + [ F \times the level of total cholesterol in mmol / L ] + [ G \times the level of triglycerides in mmol / L ] + \{ H \times [ the level of ceramide ( d 18 : 1 / 24 : 0 ) in mg / L ] - [ the level of ceramide ( d 18 : 1 / 24 : 1 ) in mg / L ] \} \qquad Formula III$$

wherein:
E is a number in the range of from about 10 to about 12 (and all one ten thousandth integers therebetween);
F is a number in the range of from about −0.1 to about −0.4 (and all one ten thousandth integers therebetween);
G is a number in the range of from about −0.1 to about −0.4 (and all one ten thousandth integers therebetween); and
H is a number in the range of from about −0.1 to about −0.4 (and all one ten thousandth integers therebetween).

Suitable embodiments of E, F, G and H are discussed supra.

In particular embodiments, the method comprises determining the prognosis for the subject using Formula IV:

$$[ 10.9903 \times the level of ceramide ( d 18 : 1 / 18 : 0 ) in mg / L ] + [ - 0.2779 \times the level of total cholesterol in mmol / L ] + [ - 0.2243 \times the level of triglycerides in mmol / L ] + \{ - 0.2366 \times [ the level of ceramide ( d 18 : 1 / 24 : 0 ) in mg / L ] - [ the level of ceramide ( d 18 : 1 / 24 : 1 ) in mg / L ] \} \qquad Formula IV .$$

A likelihood of a poor prognosis may be indicated when a score of greater than or equal to about −0.5 to about −1.5 (and all one thousandth integers therebetween) is calculated using Formula III or IV; especially a score of greater than or equal to about −0.9 to about −0.75; more especially a score of equal to or greater than about −0.817. Scores of less than this value indicate a likelihood of a good prognosis, especially a score of less than about −0.817.

In particular embodiments, a score of greater than or equal to about −0.817 calculated using Formula IV indicates a likelihood of a poor prognosis, and a score of less than about −0.817 calculated using Formula IV indicates a likelihood of a good prognosis.

Suitable subjects are as discussed above. For example, in some embodiments, the subject has undergone, is undergoing or is commencing a treatment regimen for treating prostate cancer, such as chemotherapy (e.g. docetaxel, cabazitaxel, mitoxantrone, estramustine or carboplatin), administration of an androgen receptor signalling inhibitor (e.g. abiraterone or enzalutamide), and/or administration of a poly-ADP-ribose polymerase (PARP) inhibitor (e.g. olaparib, rucaparib or niraparib).

The method may further comprise administering a treatment for prostate cancer to a subject determined to have a poor prognosis. Suitable treatments are discussed supra, such as a lipid targeted therapy (e.g. a statin, a PCSK9 inhibitor, a sphingosine kinase 1 and/or 2 inhibitor, a Des1 inhibitor and/or a SREBP inhibitor), for example, evolocumab or opaganib.

Suitable techniques for measuring or assessing the levels of the biomarkers are discussed above. In some embodiments, the levels of the biomarkers are quantitative levels. In particular embodiments, the levels of the biomarkers are determined using mass spectrometry; especially using absolute quantification as discussed supra.

The invention also provides the use of the biomarkers for determining or predicting the likelihood of survival of a subject with prostate cancer. Further provided herein is a method for determining or predicting the likelihood of survival of a subject with prostate cancer, the method comprising, consisting or consisting essentially of:
a) determining the level of at least three lipid biomarkers in a biological sample obtained from the subject, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0); and
b) comparing the level of the biomarkers to the respective levels of the corresponding biomarkers in a reference sample; and
c) determining or predicting the likelihood of survival of the subject based on the determined levels of the biomarkers and said comparison.

Suitable reference samples include, for example, a control biological sample obtained from a reference population of subjects with prostate cancer having a favourable outcome (e.g. no disease progression and/or no death from disease within about 18 months from commencement of chemotherapy or treatment with an androgen receptor signalling inhibitor).

Suitable types of prostate cancer and subjects are discussed supra. In particular embodiments, the prostate cancer is castration-resistant prostate cancer. In some embodiments, the cancer is a metastatic cancer, such as metastatic castration-resistant prostate cancer.

In exemplary embodiments, the lipid biomarkers are selected from the group consisting of ceramide (d18:1/18:0), ceramide (d18:1/22:0), ceramide (d18:1/24:0), ceramide (d18:1/24:1), ceramide (d20:1/24:1) and phosphatidylcholine (16:0/16:0); especially wherein the lipid biomarkers are selected from the group consisting of ceramide (d18:1/18:0), ceramide (d18:1/22:0), ceramide (d18:1/24:0), ceramide (d18:1/24:1) and ceramide (d20:1/24:1). In particular embodiments, the lipid biomarkers are ceramide (d18:1/18:0), ceramide (d18:1/24:0) and ceramide (d18:1/24:1).

Step a) may further comprise determining the level of one or more biomarkers selected from the group consisting of total cholesterol, triglycerides and high-density lipoprotein; especially the level of one or more biomarkers selected from the group consisting of total cholesterol and triglycerides. In particular embodiments, step a) further comprises determining the level of total cholesterol and triglycerides. In preferred embodiments, step a) comprises determining the level of ceramide (d18:1/18:0), ceramide (d18:1/24:0), ceramide (d18:1/24:1), total cholesterol and triglycerides.

In an alternative aspect, step a) comprises determining the level of at least three biomarkers in a biological sample obtained from the subject, wherein the biomarkers comprise at least one biomarker selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0); and at least two biomarkers selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0), sphingomyelin (d18:1/16:0), total cholesterol, triglycerides and high-density lipoprotein. In particular embodiments, step a) comprises determining the level of ceramide (d18:1/18:0), ceramide (d18:1/24:0), ceramide (d18:1/24:1), total cholesterol and triglycerides.

In some embodiments, the method comprises determining the level of ceramide (d18:1/18:0), ceramide (d18:1/24:0), total cholesterol and triglycerides; ceramide (d18:1/18:0), ceramide (d18:1/24:0) and ceramide (d20:1/24:1); ceramide (d18:1/18:0), ceramide (d18:1/24:1), ceramide (d18:1/22:0), total cholesterol and triglycerides; ceramide (d18:1/18:0), ceramide (d18:1/22:0), ceramide (d18:1/24:0), ceramide (d18:1/24:1), ceramide (d20:1/24:0), ceramide (d20:1/24:1) and total cholesterol; ceramide (d18:1/18:0), ceramide (d18:1/22:0), ceramide (d18:1/24:0), ceramide (d18:1/24:1), ceramide (d20:1/24:0) and ceramide (d20:1/24:1); ceramide (d18:1/18:0), ceramide (d18:1/24:1), phosphatidylcholine (16:0/16:0) and total cholesterol; ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and total cholesterol; or ceramide (d18:1/18:0), ceramide (d18:1/24:0), ceramide (d18:1/24:1) and total cholesterol.

The biological sample may be any sample that contains the biomarkers as discussed in detail elsewhere herein, such as blood, serum or plasma; especially plasma.

Survival includes no death from the prostate cancer within a certain period of time, such as from about 6 to about 36 months (and all integer months therebetween), including about 6, 8, 10, 12, 14, 16, 18, 20, 22, 24, 26, 28, 30, 32, 34 or 36 months; especially about 18 months. In particular embodiments, survival comprises no death from disease within about 18 months from commencement of chemotherapy or treatment with an androgen receptor signalling inhibitor. In such embodiments, non-survival comprises death from disease within about 18 months from commencement of chemotherapy or treatment with an androgen receptor signalling inhibitor.

In some embodiments, the reference sample is a control biological sample obtained from a reference population of subjects with prostate cancer having a favourable outcome and the subject has a likelihood of non-survival if: the level of ceramide (d18:1/18:0) is higher than in the reference sample; the level of ceramide (d18:1/22:0) is lower than in the reference sample; the level of ceramide (d18:1/24:0) is lower than in the reference sample; the level of ceramide (d18:1/24:1) is higher than in the reference sample; the level of ceramide (d20:1/24:0) is lower than in the reference sample; the level of ceramide (d20:1/24:1) is higher than in the reference sample; and/or the level of phosphatidylcholine (16:0/16:0) is higher than in the reference sample. In particular embodiments, step a) further comprises determining the level of one or more biomarkers selected from the group consisting of total cholesterol, triglycerides and high-density lipoprotein, and the subject has a likelihood of non-survival if: the level of total cholesterol is lower than in the reference sample; the level of triglycerides is lower than in the reference sample; and/or the level of high-density lipoprotein is lower than in the reference sample.

In some embodiments, the method comprises determining the likelihood of survival by dividing the level of ceramide (d18:1/24:0) by the level of ceramide (d18:1/24:1), and adding this value to the level of ceramide (d18:1/18:0), the level of total cholesterol, and the level of triglycerides, or functionalised levels of any of the foregoing levels. For example, in some embodiments, the method comprises determining the indicator using Formula II:

$$[ A \times the level of ceramide ( d 18 : 1 / 18 : 0 ) in mg / L ] + [ B \times the level of total cholesterol in mmol / L ] + [ C \times the level of triglycerides in mmol / L ] + [ D \times ( the level of ceramide ( d 18 : 1 / 24 : 0 ) in mg / L / the level of ceramide ( d 18 : 1 / 24 : 1 ) in mg / L ) ] \qquad Formula II$$

wherein:
A is a number in the range of from about 9 to about 11 (and all one ten thousandth integers therebetween);
B is a number in the range of from about −0.1 to about −0.4 (and all one ten thousandth integers therebetween);
C is a number in the range of from about −0.1 to about −0.4 (and all one ten thousandth integers therebetween); and
D is a number in the range of from about −0.1 to about −0.4 (and all one ten thousandth integers therebetween).

Suitable embodiments of A, B, C and D are discussed supra.

In particular embodiments, the method comprises determining the likelihood of survival using Formula I:

$$[ 10.0506 \times the level of ceramide ( d 18 : 1 / 18 : 0 ) in mg / L ] + [ - 0.2783 \times the level of total cholesterol in mmol / L ] + [ - 0.224 \times the level of triglycerides in mmol / L ] + [ - 0.2979 \times ( the level of ceramide ( d 18 : 1 / 24 : 0 ) in mg / L / the level of ceramide ( d 18 : 1 / 24 : 1 ) in mg / L ) ] \qquad Formula I .$$

A likelihood of non-survival may be indicated when a score of greater than or equal to about −0.5 to about −2 (and all one ten thousandth integers therebetween) is calculated using Formula I or II; especially a score of greater than or equal to about −1.3 to about −1.16; more especially a score of equal to or greater than about −1.1903. Scores of less than this value indicate a likelihood of survival, especially a score of less than about −1.1903.

In specific embodiments, a score of greater than or equal to about −1.1903 calculated using Formula I indicates a likelihood of non-survival, and a score of less than about −1.1903 calculated using Formula I indicates a likelihood of survival.

In alternative embodiments, the method comprises determining the likelihood of survival by subtracting the level of ceramide (d18:1/24:1) from the level of ceramide (d18:1/24:0), and adding this value to the level of ceramide (d18:1/18:0), the level of total cholesterol, and the level of triglycerides, or functionalised levels of any of the foregoing levels. In some embodiments, the method comprises determining the indicator using Formula III:

$$[ E \times the level of ceramide ( d 18 : 1 / 18 : 0 ) in mg / L ] + [ F \times the level of total cholesterol in mmol / L ] + [ G \times the level of triglycerides in mmol / L ] + \{ H \times [ the level of ceramide ( d 18 : 1 / 24 : 0 ) in mg / L ] - [ the level of ceramide ( d 18 : 1 / 24 : 1 ) in mg / L ] \} \qquad Formula III$$

wherein:
E is a number in the range of from about 10 to about 12 (and all one ten thousandth integers therebetween);
F is a number in the range of from about −0.1 to about −0.4 (and all one ten thousandth integers therebetween);
G is a number in the range of from about −0.1 to about −0.4 (and all one ten thousandth integers therebetween); and
H is a number in the range of from about −0.1 to about −0.4 (and all one ten thousandth integers therebetween).

Suitable embodiments of E, F, G and H are discussed supra.

In particular embodiments, the method comprises determining the likelihood of survival using Formula IV:

$$[ 10.9903 \times the level of ceramide ( d 18 : 1 / 18 : 0 ) in mg / L ] + [ - 0.2779 \times the level of total cholesterol in mmol / L ] + [ - 0.2243 \times the level of triglycerides in mmol / L ] + \{ - 0.2366 \times [ the level of ceramide ( d 18 : 1 / 24 : 0 ) in mg / L ] - [ the level of ceramide ( d 18 : 1 / 24 : 1 ) in mg / L ] \} \qquad Formula IV .$$

A likelihood of non-survival may be indicated when a score of greater than or equal to about −0.5 to about −1.5 (and all one thousandth integers therebetween) is calculated using Formula III or IV; especially a score of greater than or equal to about −0.9 to about −0.75; more especially a score of equal to or greater than about −0.817. Scores of less than this value indicate a likelihood of survival, especially a score of less than about −0.817.

In particular embodiments, a score of greater than or equal to about −0.817 calculated using Formula IV indicates a likelihood of non-survival, and a score of less than about −0.817 calculated using Formula IV indicates a likelihood of survival.

As discussed above in detail, the subject may have undergone, is undergoing or is commencing a treatment regimen for treating prostate cancer, such as chemotherapy (e.g. docetaxel, cabazitaxel, mitoxantrone, estramustine or carboplatin), administration of an androgen receptor signalling inhibitor (e.g. abiraterone or enzalutamide), and/or administration of a poly-ADP-ribose polymerase (PARP) inhibitor (e.g. olaparib, rucaparib or niraparib). Subjects determined to have a likelihood of non-survival (e.g. death within about 18 months), may be administered a treatment for prostate cancer. Suitable treatments are discussed supra, such as a lipid targeted therapy (e.g. a statin, a PCSK9 inhibitor, a sphingosine kinase 1 and/or 2 inhibitor, a Des1 inhibitor and/or a SREBP inhibitor), for example, evolocumab or opaganib.

Suitable methods for determining the levels of the biomarkers are as discussed elsewhere herein. In particular embodiments, the levels of the biomarkers are determined using mass spectrometry; especially using absolute quantification.

The invention also contemplates methods for determining an indicator used in determining a likelihood of survival of a subject with prostate cancer. Such methods comprise, consist or consist essentially of:
a) determining the level of at least three lipid biomarkers in a biological sample obtained from the subject, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0); and
b) determining the indicator using the biomarker levels.

Suitable types of prostate cancer and biological samples are as discussed above.

In particular embodiments, the lipid biomarkers are selected from the group consisting of ceramide (d18:1/18:0), ceramide (d18:1/22:0), ceramide (d18:1/24:0), ceramide (d18:1/24:1), ceramide (d20:1/24:1) and phosphatidylcholine (16:0/16:0); especially wherein the lipid biomarkers are selected from the group consisting of ceramide (d18:1/18:0), ceramide (d18:1/22:0), ceramide (d18:1/24:0), ceramide (d18:1/24:1) and ceramide (d20:1/24:1). In specific embodiments, the lipid biomarkers are ceramide (d18:1/18:0), ceramide (d18:1/24:0) and ceramide (d18:1/24:1).

In exemplary embodiments, step a) further comprises determining the level of one or more biomarkers selected from the group consisting of total cholesterol, triglycerides and high-density lipoprotein; especially the level of one or more biomarkers selected from the group consisting of total cholesterol and triglycerides. In specific embodiments, step a) further comprises determining the level of total cholesterol and triglycerides. In particular embodiments, step a) comprises determining the level of ceramide (d18:1/18:0), ceramide (d18:1/24:0), ceramide (d18:1/24:1), total cholesterol and triglycerides.

Alternatively, step a) may comprise determining the level of at least three biomarkers in a biological sample obtained from the subject, wherein the biomarkers comprise at least one biomarker selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0); and at least two biomarkers selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0), sphingomyelin (d18:1/16:0), total cholesterol, triglycerides and high-density lipoprotein. In particular embodiments, step a) comprises determining the level of ceramide (d18:1/18:0), ceramide (d18:1/24:0), ceramide (d18:1/24:1), total cholesterol and triglycerides.

The method may comprise determining the levels of particular combinations of the biomarkers. For example, in some embodiments, the method comprises determining the level of ceramide (d18:1/18:0), ceramide (d18:1/24:0), total cholesterol and triglycerides; ceramide (d18:1/18:0), ceramide (d18:1/24:0) and ceramide (d20:1/24:1); ceramide (d18:1/18:0), ceramide (d18:1/24:1), ceramide (d18:1/22:0), total cholesterol and triglycerides; ceramide (d18:1/18:0), ceramide (d18:1/22:0), ceramide (d18:1/24:0), ceramide (d18:1/24:1), ceramide (d20:1/24:0), ceramide (d20:1/24:1) and total cholesterol; ceramide (d18:1/18:0), ceramide (d18:1/22:0), ceramide (d18:1/24:0), ceramide (d18:1/24:1), ceramide (d20:1/24:0) and ceramide (d20:1/24:1); ceramide (d18:1/18:0), ceramide (d18:1/24:1), phosphatidylcholine (16:0/16:0) and total cholesterol; ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and total cholesterol; or ceramide (d18:1/18:0), ceramide (d18:1/24:0), ceramide (d18:1/24:1) and total cholesterol.

Survival includes no death from the prostate cancer within a certain predetermined period of time, such as from about 6 to about 36 months (and all integer months therebetween), including about 6, 8, 10, 12, 14, 16, 18, 20, 22, 24, 26, 28, 30, 32, 34 or 36 months; especially about 18 months. In particular embodiments, survival comprises no death from disease within about 18 months from commencement of chemotherapy or treatment with an androgen receptor signalling inhibitor. In such embodiments, non-survival comprises death from disease within about 18 months from commencement of chemotherapy or treatment with an androgen receptor signalling inhibitor.

In exemplary embodiments, the indicator indicates a likelihood of non-survival (e.g. death from disease within about 18 months) if: the level of ceramide (d18:1/18:0) is higher than in control biological samples obtained from a reference population of subjects with prostate cancer with a favourable outcome; the level of ceramide (d18:1/22:0) is lower than in control biological samples obtained from a reference population of subjects with prostate cancer with a favourable outcome; the level of ceramide (d18:1/24:0) is lower than in control biological samples obtained from a reference population of subjects with prostate cancer with a favourable outcome; the level of ceramide (d18:1/24:1) is higher than in control biological samples obtained from a reference population of subjects with prostate cancer with a favourable outcome; the level of ceramide (d20:1/24:0) is lower than in control biological samples obtained from a reference population of subjects with prostate cancer with a favourable outcome; the level of ceramide (d20:1/24:1) is higher than in control biological samples obtained from a reference population of subjects with prostate cancer with a favourable outcome; and/or the level of phosphatidylcholine (16:0/16:0) is higher than in control biological samples obtained from a reference population of subjects with prostate cancer with a favourable outcome. In further embodiments, step a) further comprises determining the level of one or more biomarkers selected from the group consisting of total cholesterol, triglycerides and high-density lipoprotein, and the indicator indicates a likelihood of non-survival if: the level of total cholesterol is lower than in control biological samples obtained from a reference population of subjects with prostate cancer with a favourable outcome; the level of triglycerides is lower than in control biological samples obtained from a reference population of subjects with prostate cancer with a favourable outcome; and/or the level of high-density lipoprotein is lower than in control biological samples obtained from a reference population of subjects with prostate cancer with a favourable outcome.

The reference population of subjects with prostate cancer with a favourable outcome may, for example, be a population of subjects who have not died within about 6 to about 36 months (and all integer months therebetween), including about 6, 8, 10, 12, 14, 16, 18, 20, 22, 24, 26, 28, 30, 32, 34 or 36 months; especially about 18 months from commencement of chemotherapy or treatment with an androgen receptor signalling inhibitor. In particular embodiments, the reference population of subjects with a favourable outcome have not died from disease within about 18 months from commencement of chemotherapy or treatment with an androgen receptor signalling inhibitor.

The method may comprise applying a function to biomarker levels, and/or combining the biomarker levels and/or functionalised biomarker levels to provide a composite score as discussed above, which may then be compared to a reference level or score to determine the indicator.

In particular embodiments, the method comprises determining the indicator by dividing the level of ceramide (d18:1/24:0) by the level of ceramide (d18:1/24:1), and adding this value to the level of ceramide (d18:1/18:0), the level of total cholesterol, and the level of triglycerides, or functionalised levels of any of the foregoing levels. For example, in some embodiments, the method comprises determining the indicator using Formula II:

$$[ A \times the level of ceramide ( d 18 : 1 / 18 : 0 ) in mg / L ] + [ B \times the level of total cholesterol in mmol / L ] + [ C \times the level of triglycerides in mmol / L ] + [ D \times ( the level of ceramide ( d 18 : 1 / 24 : 0 ) in mg / L / the level of ceramide ( d 18 : 1 / 24 : 1 ) in mg / L ) ] \qquad Formula II$$

wherein:
A is a number in the range of from about 9 to about 11 (and all one ten thousandth integers therebetween);
B is a number in the range of from about −0.1 to about −0.4 (and all one ten thousandth integers therebetween);
C is a number in the range of from about −0.1 to about −0.4 (and all one ten thousandth integers therebetween); and
D is a number in the range of from about −0.1 to about −0.4 (and all one ten thousandth integers therebetween).

Suitable embodiments of A, B, C and D are discussed supra.

In specific embodiments, the method comprises determining the indicator using Formula I:

$$[ 10.0506 \times the level of ceramide ( d 18 : 1 / 18 : 0 ) in mg / L ] + [ - 0.2783 \times the level of total cholesterol in mmol / L ] + [ - 0.224 \times the level of triglycerides in mmol / L ] + [ - 0.2979 \times ( the level of ceramide ( d 18 : 1 / 24 : 0 ) in mg / L / the level of ceramide ( d 18 : 1 / 24 : 1 ) in mg / L ) ] \qquad Formula I .$$

A likelihood of non-survival (e.g. death from disease within about 18 months from commencement of chemotherapy or treatment with an androgen receptor signalling inhibitor) may be indicated when a score of greater than or equal to about −0.5 to about −2 (and all one ten thousandth integers therebetween) is calculated using Formula I or II; especially a score of greater than or equal to about −1.3 to about −1.16; more especially a score of equal to or greater than about −1.1903. Scores of less than this value indicate a likelihood of survival (e.g. no death from disease within about 18 months from commencement of chemotherapy or treatment with an androgen receptor signalling inhibitor), especially a score of less than about −1.1903.

In particular embodiments, a score of greater than or equal to about −1.1903 calculated using Formula I indicates a likelihood of non-survival (e.g. death from disease within about 18 months from commencement of chemotherapy or treatment with an androgen receptor signalling inhibitor), and a score of less than about −1.1903 calculated using Formula I indicates a likelihood of survival (e.g. no death from disease within about 18 months from commencement of chemotherapy or treatment with an androgen receptor signalling inhibitor).

In alternative embodiments, the method comprises determining the indicator by subtracting the level of ceramide (d18:1/24:1) from the level of ceramide (d18:1/24:0), and adding this value to the level of ceramide (d18:1/18:0), the level of total cholesterol, and the level of triglycerides, or functionalised levels of any of the foregoing levels. In some embodiments, the method comprises determining the indicator using Formula III:

$$[ E \times the level of ceramide ( d 18 : 1 / 18 : 0 ) in mg / L ] + [ F \times the level of total cholesterol in mmol / L ] + [ G \times the level of triglycerides in mmol / L ] + \{ H \times [ the level of ceramide ( d 18 : 1 / 24 : 0 ) in mg / L ] - [ the level of ceramide ( d 18 : 1 / 24 : 1 ) in mg / L ] \} \qquad Formula III$$

wherein:
E is a number in the range of from about 10 to about 12 (and all one ten thousandth integers therebetween);
F is a number in the range of from about −0.1 to about −0.4 (and all one ten thousandth integers therebetween);
G is a number in the range of from about −0.1 to about −0.4 (and all one ten thousandth integers therebetween); and
H is a number in the range of from about −0.1 to about −0.4 (and all one ten thousandth integers therebetween).

Suitable embodiments of E, F, G and H are discussed above.

In particular embodiments, the method comprises determining the indicator using Formula IV:

$$[ 10.9903 \times the level of ceramide ( d 18 : 1 / 18 : 0 ) in mg / L ] + [ - 0.2779 \times the level of total cholesterol in mmol / L ] + [ - 0.2243 \times the level of triglycerides in mmol / L ] + \{ - 0.2366 \times [ the level of ceramide ( d 18 : 1 / 24 : 0 ) in mg / L ] - [ the level of ceramide ( d 18 : 1 / 24 : 1 ) in mg / L ] \} \qquad Formula IV .$$

A likelihood of non-survival may be indicated when a score of greater than or equal to about −0.5 to about −1.5 (and all one thousandth integers therebetween) is calculated using Formula III or IV; especially a score of greater than or equal to about −0.9 to about −0.75; more especially a score of equal to or greater than about −0.817. Scores of less than this value indicate a likelihood of survival, especially a score of less than about −0.817.

In particular embodiments, a score of greater than or equal to about −0.817 calculated using Formula IV indicates a likelihood of non-survival, and a score of less than about −0.817 calculated using Formula IV indicates a likelihood of survival.

While the subject may have undergone, be undergoing or commencing a treatment regimen for treating prostate cancer, such as chemotherapy, administration of an androgen receptor signalling inhibitor and/or administration of a PARP inhibitor, a subject determined to have a likelihood of non-survival may be administered a treatment for prostate cancer. Suitable treatments are discussed above. In particular embodiments, the treatment comprises administering a lipid targeted therapy, such as evolocumab or opaganib.

Suitable methods for determining the levels of the biomarkers are discussed elsewhere herein. In specific embodiments, the levels of the biomarkers are determined using mass spectrometry; especially using absolute quantification.

The methods of the invention may also be useful for monitoring the prognosis for a subject over a particular time period, such as one week, one month or for several years. This may involve determining the level of the biomarkers disclosed herein in biological samples taken from a single subject at two separate time points [e.g. from one week to three years (and all integer weeks and months therebetween)] and comparing the levels between the two samples. A change in the levels of the biomarkers between the samples is indicative of a change in the prognosis for the subject.

Accordingly, in another aspect, there is provided a method for monitoring the prognosis for a subject with prostate cancer, the method comprising, consisting or consisting essentially of:
a) determining the level of at least three lipid biomarkers in a first biological sample obtained from the subject, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0);
b) determining a first indicator using the biomarker levels;
c) determining the level of at least three lipid biomarkers in a second biological sample obtained from the subject, wherein the at least three lipid biomarkers are selected from the group consisting of ceramide (d18:1/18:0), ceramide (d18:1/22:0), ceramide ceramide (d18:1/24:1), ceramide (d20:1/24:0), ceramide (d18:1/24:0), (d20:1/24:1), phosphatidylcholine (16:0/16:0) and sphingomyelin (d18:1/16:0), and the second biological sample is obtained at a time point after the first biological sample is obtained;
d) determining a second indicator using the biomarker levels; and
e) comparing the indicator in the first and second biological samples;
wherein a change in the indicator between the first and second biological samples is indicative of a change in the prognosis for the subject.

Suitable embodiments of the method are as discussed above, including prostate cancer types, biological samples and methods for determining the biomarker levels, the indicator and the prognosis for the subject.

In some embodiments, the lipid biomarkers in steps a) and c) are selected from the group consisting of ceramide (d18:1/18:0), ceramide (d18:1/22:0), ceramide (d18:1/24:0), ceramide (d18:1/24:1), ceramide (d20:1/24:1) and phosphatidylcholine (16:0/16:0); especially wherein the lipid biomarkers in steps a) and c) are selected from the group consisting of ceramide (d18:1/18:0), ceramide (d18:1/22:0), ceramide (d18:1/24:0), ceramide (d18:1/24:1) and ceramide (d20:1/24:1). In particular embodiments, the lipid biomarkers in steps a) and c) are ceramide (d18:1/18:0), ceramide (d18:1/24:0) and ceramide (d18:1/24:1).

The method may comprise, as discussed in detail elsewhere herein, determining the level of one or more additional biomarkers in steps a) and c), such as total cholesterol, triglycerides and/or high-density lipoprotein; especially the level of one or more biomarkers selected from the group consisting of total cholesterol and triglycerides. In specific embodiments, steps a) and c) further comprise determining the level of total cholesterol and triglycerides. In particular embodiments, steps a) and c) comprise determining the level of ceramide (d18:1/18:0), ceramide (d18:1/24:0), ceramide (d18:1/24:1), total cholesterol and triglycerides.

Alternatively, steps a) and c) may comprise determining the level of at least three biomarkers in a biological sample obtained from the subject, wherein the biomarkers comprise at least one biomarker selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0); and at least two biomarkers selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0), sphingomyelin (d18:1/16:0), total cholesterol, triglycerides and high-density lipoprotein. In particular embodiments, step a) comprises determining the level of ceramide (d18:1/18:0), ceramide (d18:1/24:0), ceramide (d18:1/24:1), total cholesterol and triglycerides.

The method may comprise determining the levels of particular combinations of the biomarkers. For example, in some embodiments, the method comprises determining the level of ceramide (d18:1/18:0), ceramide (d18:1/24:0), total cholesterol and triglycerides; ceramide (d18:1/18:0), ceramide (d18:1/24:0) and ceramide (d20:1/24:1); ceramide (d18:1/18:0), ceramide (d18:1/24:1), ceramide (d18:1/22:0), total cholesterol and triglycerides; ceramide (d18:1/18:0), ceramide (d18:1/22:0), ceramide (d18:1/24:0), ceramide (d18:1/24:1), ceramide (d20:1/24:0), ceramide (d20:1/24:1) and total cholesterol; ceramide (d18:1/18:0), ceramide (d18:1/22:0), ceramide (d18:1/24:0), ceramide (d18:1/24:1), ceramide (d20:1/24:0) and ceramide (d20:1/24:1); ceramide (d18:1/18:0), ceramide (d18:1/24:1), phosphatidylcholine (16:0/16:0) and total cholesterol; ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and total cholesterol; or ceramide (d18:1/18:0), ceramide (d18:1/24:0), ceramide (d18:1/24:1) and total cholesterol.

If the prognosis for the subject has changed to a likelihood of a poor prognosis, the subject may be administered a treatment for prostate cancer, such as a lipid targeted therapy. Suitable treatments are as discussed supra. Alternatively, if the prognosis of the subject has changed to a good prognosis, the current treatment regime for the subject may be reconsidered and/or ceased (e.g. if the subject is being administered a lipid targeted therapy).

In any one of the aspects described above, biomarker data may be analysed by a variety of methods to identify the biomarkers and determine the statistical significance of differences in observed levels of the biomarkers between test and reference samples and/or populations in order to evaluate whether the subject has a likelihood of a good prognosis, poor prognosis, survival and non-survival as discussed herein. For any particular biomarker, a distribution of biomarker levels or abundances for poor outcome patients and good outcome patients will likely overlap. Under such conditions, a test does not absolutely distinguish the different outcomes with 100% accuracy, and the area of overlap indicates where the test cannot distinguish the poor outcome and the good outcome. A threshold is selected, above which (or below which, depending on how the biomarker(s) change with a specified prognosis) the test is considered to be “positive” and below which the test is considered to be “negative.” The area under the ROC curve (AUC) provides the C-statistic, which is a measure of the probability that the perceived measurement will allow correct identification of a condition (refer to, for example, Hanley et al. (1982) Radiology, 143:29-36).

Alternatively, or in addition, thresholds may be established by obtaining an earlier biomarker result from the same subject, to which later results may be compared. In these embodiments, the individual in effect acts as their own “control group.” For biomarkers that decrease inversely with prognostic risk, a decrease over time in the same subject can indicate a worsening of the cancer, a failure of a treatment regimen or a poor outcome, while an increase over time can indicate remission of the cancer, success of a treatment regimen or a good outcome.

In some embodiments, a positive likelihood ratio, negative likelihood ratio, odds ratio and/or AUC or receiver operating characteristic (ROC) values are used as a measure of a method's ability to prognose subject outcome. As used herein, the term “likelihood ratio” is the probability that a given test result would be observed in a subject with a particular prognostic outcome divided by the probability that that same result would be observed in a patient without the prognostic outcome. Thus, a positive likelihood ratio is the probability of a positive result observed in subjects with the specified prognostic outcome, divided by the probability of a positive results in subjects without the specified prognostic outcome. A negative likelihood ratio is the probability of a negative result in subjects without the specified prognostic outcome divided by the probability of a negative result in subjects with specified prognostic outcome. The term “odds ratio,” as used herein, refers to the ratio of the odds of an event occurring in one group (e.g. one of the prognostic outcomes discloses herein) to the odds of it occurring in another group (e.g. another of the disclosed prognostic outcomes), or to a data-based estimate of that ratio. The term “area under the curve” or “AUC” refers to the area under the curve of a receiver operating characteristic (ROC) curve, both of which are well known in the art. AUC measures are useful for comparing the accuracy of a classifier across the complete data range. Classifiers with a greater AUC have a greater capacity to classify unknowns correctly between two groups of interest (e.g. a first disclosed prognostic outcome such as a poor prognosis and a second disclosed prognostic outcome such as good prognosis). ROC curves are useful for plotting the performance of a particular feature (e.g. any of the biomarkers disclosed herein and/or any item clinical parameter or symptom information) in distinguishing or discriminating between two populations (e.g. a first disclosed prognostic outcome and a second disclosed prognostic outcome). Typically, the feature data across the entire population (e.g. subjects with a first disclosed prognostic outcome and subjects with a second disclosed prognostic outcome) are sorted in ascending order based on the value of a single feature. Then, for each value for that feature, the true positive and false positive rates for the data are calculated. The sensitivity is determined by counting the number of cases above the value for that feature and then dividing by the total number of cases. The specificity is determined by counting the number of controls below the value for that feature and then dividing by the total number of controls. Alternatively, specificity may be calculated by ROC curve and threshold value. Although this definition refers to scenarios in which a feature is elevated in one subject group compared to another subject group, this definition also applies to scenarios in which a feature is lower in one subject group compared to the other subject group (in such a scenario, samples below the value for that feature would be counted). ROC curves can be generated for a single feature as well as for other single outputs, for example, a combination of two or more features (e.g. a combination of two or more biomarker levels) can be mathematically combined (e.g. added, subtracted, multiplied, etc.) to produce a single value, and this single value can be plotted in a ROC curve. Additionally, any combination of multiple features (e.g. a combination of multiple biomarker levels), in which the combination derives a single output value, can be plotted in a ROC curve. These combinations of features may comprise a test. The ROC curve is the plot of the sensitivity of a test against the specificity of the test, where sensitivity is traditionally presented on the vertical axis and specificity is traditionally presented on the horizontal axis. Thus, “AUC ROC values” are equal to the probability that a classifier will rank a randomly chosen positive instance higher than a randomly chosen negative one. An AUC ROC value may be thought of as equivalent to the Mann-Whitney U test, which tests for the median difference between scores obtained in the two groups considered if the groups are of continuous data or to the Wilcoxon test of ranks.

In some embodiments, a biomarker or a panel of biomarkers is selected to discriminate between subjects with a first disclosed prognostic outcome and subjects with a second disclosed prognostic outcome, with at least about 50%, 55% 60%, 65%, 70%, 75%, 80%, 85%, 90% or 95% accuracy or having a C-statistic of at least about 0.50, 0.55, 0.60, 0.65, 0.70, 0.75, 0.80, 0.85, 0.90 or 0.95.

In the case of a positive likelihood ratio, a value of 1 indicates that a positive result is equally likely among subjects in both the “first condition” and “second condition” groups, a value greater than 1 indicates that a positive result is more likely in the first condition group, and a value less than 1 indicates that a positive result is more likely in the second condition group. In this context, “first condition” group is meant to refer to a group having one characteristic (e.g. a first disclosed prognostic outcome) and “second condition” group (e.g. a second disclosed prognostic outcome) lacking the same characteristic. In the case of a negative likelihood ratio, a value of 1 indicates that a negative result is equally likely among subjects in both the “first condition” and “second condition” groups, a value greater than 1 indicates that a negative result is more likely in the “first condition” group, and a value less than 1 indicates that a negative result is more likely in the “second condition” group. In the case of an odds ratio, a value of 1 indicates that a positive result is equally likely among subjects in both the “first condition” and “second condition” groups, a value greater than 1 indicates that a positive result is more likely in the “first condition” group, and a value less than 1 indicates that a positive result is more likely in the “second condition” group. In the case of an AUC ROC value, this is computed by numerical integration of the ROC curve. The range of this value can be 0.5 to 1.0. A value of 0.5 indicates that a classifier (e.g. a biomarker signature) is no better than a 50% chance to classify unknowns correctly between two groups of interest (e.g. a first disclosed prognostic outcome and a second disclosed prognostic outcome disclosed herein), while 1.0 indicates the relatively best diagnostic accuracy. In certain embodiments, individual biomarkers and/or biomarker panels are selected to exhibit a positive or negative likelihood ratio of at least about 1.5 or more or about 0.67 or less, at least about 2 or more or about 0.5 or less, at least about 5 or more or about 0.2 or less, at least about 10 or more or about 0.1 or less, or at least about 20 or more or about 0.05 or less.

In certain embodiments, individual biomarkers and/or biomarker panels are selected to exhibit an odds ratio of at least about 2 or more or about 0.5 or less, at least about 3 or more or about 0.33 or less, at least about 4 or more or about 0.25 or less, at least about 5 or more or about 0.2 or less, or at least about 10 or more or about 0.1 or less.

In some embodiments, individual biomarkers and/or biomarker panels are selected to exhibit an AUC ROC value of greater than 0.5, preferably at least 0.6, more preferably 0.7, still more preferably at least 0.8, even more preferably at least 0.9 and most preferably at least 0.95.

In some cases, multiple thresholds may be determined in so-called “tertile,” “quartile,” or “quintile” analyses. In these methods, the “diseased” and “control groups” (or “high risk” and “low risk”) groups are considered together as a single population, and are divided into 3, 4, or 5 (or more) “bins” having equal numbers of individuals. The boundary between two of these “bins” may be considered a “threshold.” A risk (of a particular prognosis for example) can be assigned based on which “bin” a test subject falls into.

In other embodiments, particular thresholds for the protein biomarker(s) measured are not relied upon to determine if the biomarker level(s) obtained from a subject are correlated to a particular prognosis. For example, a temporal change in the biomarker(s) can be used to rule in or out one or more particular prognoses. Alternatively, biomarker(s) may be correlated to a prognosis by the presence or absence of one or more biomarkers in a particular assay format. In the case of biomarker panels, the detection methods disclosed herein may utilise an evaluation of the entire population or subset of biomarkers disclosed herein to provide a single result value (e.g. a “panel response” value expressed either as a numeric score or as a percentage risk).

In particular embodiments, a panel of biomarkers is selected to assist in distinguishing a pair of groups (i.e. assist in assessing whether a subject has an increased likelihood of being in one group or the other group of the pair) selected from a “favourable outcome group” (e.g. a good prognosis, such as no disease progression and/or death from disease within about 18 months from commencement of chemotherapy or treatment with an androgen receptor signalling inhibitor) and an “unfavourable outcome group” (e.g. a poor prognosis, such as disease progression and/or death from disease within about 18 months from commencement of chemotherapy or treatment with an androgen receptor signalling inhibitor), or “low risk” and “high risk” with at least about 70%, 80%, 85%, 90% or 95% sensitivity, suitably in combination with at least about 70% 80%, 85%, 90% or 95% specificity. In some embodiments, both the sensitivity and specificity are at least about 75%, 80%, 85%, 90% or 95%.

The phrases “assessing the likelihood” and “determining the likelihood,” as used herein, refer to methods by which the skilled person can predict a favourable outcome and an unfavourable outcome in a subject, such as a good prognosis, poor prognosis, survival or non-survival. The skilled person will understand that this phrase includes within its scope an increased probability that the patient has one of the disclosed prognoses; that is, that the patient is more likely to have the prognosis. For example, the probability that an individual predicted to have a specified prognosis (e.g. a good prognosis or a poor prognosis) actually has the prognosis may be expressed as a “positive predictive value” or “PPV.” Positive predictive value can be calculated as the number of true positives divided by the sum of the true positives and false positives. PPV is determined by the characteristics of the predictive methods disclosed herein as well as the prevalence of the condition in the population analysed. The statistical algorithms can be selected such that the positive predictive value in a population having a condition prevalence is in the range of about 70% to about 99% and can be, for example, at least about 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In other examples, the probability that an individual predicted as not having a specified prognosis actually does not have that prognosis may be expressed as a “negative predictive value” or “NPV.” Negative predictive value can be calculated as the number of true negatives divided by the sum of the true negatives and false negatives. Negative predictive value is determined by the characteristics of the prognostic method as well as the prevalence of the disease in the population analysed. The statistical methods and models can be selected such that the negative predictive value in a population having a condition prevalence is in the range of about 70% to about 99% and can be, for example, at least about 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

In some embodiments, a subject is determined as having a significant likelihood of having or not having a specified prognosis (e.g. a good prognosis or a poor prognosis as disclosed herein). By “significant likelihood” is meant that the subject has a reasonable probability (e.g. about 0.6, 0.7, 0.8, 0.9 or more) of having, or not having, a specified prognosis.

The protein biomarker analysis disclosed herein permits the generation of high-density data sets that can be evaluated using informatics approaches. High data density informatics analytical methods are known and software is available to those in the art, e.g., cluster analysis (Pirouette, Informetrix), class prediction (SIMCA-P, Umetrics), principal components analysis of a computationally modeled dataset (SIMCA-P, Umetrics), 2D cluster analysis (GeneLinker Platinum, Improved Outcomes Software), and metabolic pathway analysis (biotech.icmb.utexas.edu). The choice of software packages offers specific tools for questions of interest (Kennedy et al. (1997) Solving Data Mining Problems Through Pattern Recognition. Indianapolis: Prentice Hall PTR; Golub et al., (1999) Science 286: 531-537; Eriksson et al. (2001) Multi and Megavariate Analysis Principles and Applications: Umetrics, Umea). In general, any suitable mathematic analyses can be used to evaluate at least one biomarker in a population disclosed herein with respect to a disclosed prognosis. For example, methods such as multivariate analysis of variance, multivariate regression, and/or multiple regression can be used to determine relationships between dependent variables (e.g. clinical measures) and independent variables (e.g. levels of biomarkers). Clustering, including both hierarchical and non-hierarchical methods, as well as non-metric Dimensional Scaling can be used to determine associations or relationships among variables and among changes in those variables.

In addition, principal component analysis is a common way of reducing the dimension of studies, and can be used to interpret the variance-covariance structure of a data set. Principal components may be used in such applications as multiple regression and cluster analysis. Factor analysis is used to describe the covariance by constructing “hidden” variables from the observed variables. Factor analysis may be considered an extension of principal component analysis, where principal component analysis is used as parameter estimation along with the maximum likelihood method. Furthermore, simple hypothesis such as equality of two vectors of means can be tested using Hotelling's T squared statistic.

In some embodiments, the data sets corresponding to biomarker panels disclosed herein are used to create a predictive rule or model based on the application of a statistical and machine learning algorithm. Such an algorithm uses relationships between a biomarker panel and a disclosed prognosis (e.g. a good prognosis or a poor prognosis as disclosed herein), observed in control subjects or typically cohorts of control subjects (sometimes referred to as training data), which provides combined control or reference biomarker panels for comparison with biomarker panels of a subject. The data are used to infer relationships that are then used to predict the prognosis or survival of a subject.

Practitioners skilled in the art of data analysis will recognise that many different forms of inferring relationships in the training data may be used without materially changing the detection methods disclosed herein. The data presented in the Tables, Examples and Figures herein have been used to generate illustrative minimal combinations of biomarkers (models) that differentiate between the disclosed prognoses (i.e. a good prognosis or a poor prognosis and survival and non-survival as disclosed herein). Illustrative models comprising at least three biomarkers were able to develop a classifier or generative algorithm (e.g. Formula I) for discriminating between two control groups as defined above with significantly improved positive predictive values compared to conventional methodologies. This algorithm can be advantageously applied to determine the probability of one of the prognoses disclosed herein in a subject, and thus prognose the subject as having decreased or poor survival prognosis, or as having increased or good survival prognosis.

In some embodiments, evaluation of biomarkers includes determining the levels of individual protein biomarkers, which correlate with a prognosis, as defined above. In certain embodiments, the techniques used for detection of protein biomarkers may include internal or external standards to permit quantitative or semi-quantitative determination of those biomarkers, to thereby enable a valid comparison of the level of the biomarkers in a biological sample with the corresponding biomarkers in a reference sample or samples. Such standards can be determined by the skilled person using standard protocols. In specific examples, absolute values for the level or functional activity of individual expression products are determined. Exemplary internal standards are discussed in further detail supra.

In semi-quantitative methods, a threshold or cut-off value is suitably determined and is optionally a predetermined value. In particular embodiments, the threshold value is predetermined in the sense that it is fixed, for example, based on previous experience with the assay and/or a population of affected and/or unaffected subjects. Alternatively, the predetermined value can also indicate that the method of arriving at the threshold is predetermined or fixed even if the particular value varies among assays or may even be determined for every assay run.

In some embodiments, the level of a biomarker is normalised. There is no intended limitation on the methodology used to normalise the values of the measured biomarkers provided that the same methodology is used for testing a human subject sample as was used to generate a risk categorisation table or threshold value. Many methods for data normalisation exist and are familiar to those skilled in the art. These include methods such as background subtraction, scaling, MoM analysis, linear transformation, least squares fitting, etc. The goal of normalisation is to equate the varying measurement scales for the separate biomarkers such that the resulting values may be combined according to a weighting scale as determined and designed by the user or by the machine learning system and are not influenced by the absolute or relative values of the biomarker found within nature.

Composite scores may be calculated using standard statistical analysis well known to one of skill in the art wherein the measurements of each biomarker in the panel are combined, optionally with clinical parameters, to provide a probability value. For example, generalized or multivariate logistic regression analysis may be used to derive a mathematical function with a set of variables corresponding to each biomarker and optional clinical parameter, which provides a weighting factor for each variable. The weighting factors are derived to optimise the agency of the function to predict the dependent variable, which is the dichotomy of a first prognostic outcome (e.g. a poor prognosis) as compared to a second prognostic outcome (e.g. a good prognosis) disclosed herein. The weighting factors are specific to the particular variable combination (e.g. biomarker panel analysed). The function can then be applied to the original samples to predict a probability of a disclosed prognosis or survival. In this way, a retrospective data set may be used to provide weighting factors for a particular panel of biomarkers, optionally in combination with clinical parameters, which is then used to calculate the probability of a disclosed prognosis in a patient.

Composite scores may be calculated for example using the statistical methodology disclosed in US Publ. No. 2008/013314 for handling and interpreting data from a multiplex assay. In this methodology, the amount of any one biomarker is compared to a predetermined cut-off distinguishing positive from negative for that biomarker as determined from a control population study of patients with a prognostic outcome (e.g. poor prognosis) and suitably matched controls (e.g. subjects with an unfavourable outcome) to yield a score for each biomarker based on that comparison; and then combining the scores for each biomarker to obtain a composite score for the biomarker(s) in the sample.

A predetermined cut-off can be based on ROC curves and the score for each biomarker can be calculated based on the specificity of the biomarker. Then, the total score can be compared to a predetermined total score to transform that total score to a qualitative determination of the likelihood or risk of having a particular prognosis as disclosed herein.

In certain embodiments, the detection methods utilise a risk categorisation table to generate a risk score for a subject based on a composite score by comparing the composite score with a reference set derived from a cohort of subjects with one of the prognostic outcomes disclosed herein. The detection methods may further comprise quantifying the increased risk for the presence of a disclosed prognostic outcome in the patient as a risk score, wherein the composite score (combined obtained biomarker levels or value and optionally obtained clinical parameter values) is matched to a risk category of a grouping of stratified subject populations wherein each risk category comprises a multiplier (or percentage) indicating an increased likelihood of having the prognostic outcome correlated to a range of composite scores. This quantification is based on the pre-determined grouping of a stratified cohort of subjects. In some embodiments, the grouping of a stratified population of subjects, or stratification of a prognosis cohort, is in the form of a risk categorization table. The selection of the prognosis cohort, the cohort of subjects that share disclosed prognostic outcome risk factors, are well understood by those skilled in the art of cancer research. However, the skilled person would also recognise that the resulting stratification, may be more multidimensional and take into account further environmental, occupational, genetic or biological factors (e.g. epidemiological factors).

After quantifying the increased risk for presence of a disclosed prognostic outcome (e.g. a poor prognosis such as disease progression and/or death from disease within about 18 months from commencement of chemotherapy or treatment with an androgen receptor signalling inhibitor, or a good prognosis such as no disease progression and/or death from disease within about 18 months from commencement of chemotherapy or treatment with an androgen receptor signalling inhibitor) in the form of a risk score, this score may be provided in a form amenable to understanding by a physician. In certain embodiments, the risk score is provided in a report. The report may, for example, comprise one or more of the following: subject information, a risk categorisation table, a risk score relative to a cohort population, one or more biomarker test scores, a biomarker composite score, a master composite score, identification of the risk category for the subject, an explanation of the risk categorisation table, and the resulting test score, a list of biomarkers tested, a description of the disease cohort, environmental and/or occupational factors, cohort size, biomarker velocity, genetic mutations, family history, margin of error, and so on.

### 4. Kits and Compositions

The invention further provides compositions which are useful for determining or predicting prognosis for and survival of a subject with prostate cancer. Such compositions may comprise biological samples comprising the lipid biomarkers, together with reagents for identifying the presence and/or levels of the lipid biomarkers.

The composition may comprise one or more reagents that permit quantification of at least one biomarker or each biomarker disclosed herein. Suitable reagents include, but are not limited to, antibodies, or labelled biomarkers such as an isotopically-labelled biomarker (e.g. for mass spectrometry quantification). In particular embodiments, the reagent is one or more isotopically labelled internal standards for each biomarker of interest, such as D₇-ceramide (d18:1/18:0), D₇-ceramide (d18:1/22:0), D₇-ceramide (d18:1/24:0), D₇-ceramide (d18:1/24:1), D₇-ceramide (d20:1/24:0), D₇-ceramide (d20:1/24:1), D₉-phosphatidylcholine (16:0/16:0), D₉-sphingomyelin (d18:1/16:0) or D₃₁-sphingomyelin (d18:1/16:0); especially D₇-ceramide (d18:1/18:0), D₇-ceramide (d18:1/24:0), D₇-ceramide (d18:1/24:1), D₉-phosphatidylcholine (16:0/16:0), D₉-sphingomyelin (d18:1/16:0) or D₃₁-sphingomyelin (d18:1/16:0).

Accordingly, the invention further provides a composition comprising a biological sample from a subject with prostate cancer and an isotope-labelled lipid corresponding to each of at least three lipid biomarkers in the sample, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0).

In some embodiments, the subject has castration-resistant prostate cancer, especially metastatic castration-resistant prostate cancer.

In some embodiments, the lipid biomarkers are selected from the group consisting of ceramide (d18:1/18:0), ceramide (d18:1/22:0), ceramide (d18:1/24:0), ceramide (d18:1/24:1), ceramide (d20:1/24:1) and phosphatidylcholine (16:0/16:0); especially wherein the lipid biomarkers are selected from the group consisting of ceramide (d18:1/18:0), ceramide (d18:1/22:0), ceramide (d18:1/24:0), ceramide (d18:1/24:1) and ceramide (d20:1/24:1). In specific embodiments, the lipid biomarkers are ceramide (d18:1/18:0), ceramide (d18:1/24:0) and ceramide (d18:1/24:1).

The lipids may be labelled with any isotope suitable for detection via quantitative detection techniques, e.g. mass spectrometry, such as deuterium, ¹³C, ¹⁵N, and the like. In particular embodiments, the isotope is deuterium. Suitable isotope-labelled lipids corresponding to the biomarkers of the invention include, but are not limited to, D₇-ceramide (d18:1/18:0), D₇-ceramide (d18:1/22:0), D₇-ceramide (d18:1/24:0), D₇-ceramide (d18:1/24:1), D₇-ceramide (d20:1/24:0), D₇-ceramide (d20:1/24:1), D₉-phosphatidylcholine (16:0/16:0), D₉-sphingomyelin (d18:1/16:0) or D₃₁-sphingomyelin (d18:1/16:0); especially D₇-ceramide (d18:1/18:0), D₇-ceramide (d18:1/24:0), D₇-ceramide (d18:1/24:1), D₉-phosphatidylcholine (16:0/16:0), D₉-sphingomyelin (d18:1/16:0) or D₃₁-sphingomyelin (d18:1/16:0).

Suitable biological samples are discussed in detail elsewhere herein. In particular embodiments, the biological sample is blood, serum or plasma; especially plasma.

The reagents and samples required for detecting the biomarkers disclosed herein may be assembled together in a kit. For example, the kit may comprise one or more reagents that permit quantification of at least one biomarker or each biomarker disclosed herein. Suitable reagents are as discussed above. In particular embodiments, the one or more reagents is a labelled biomarker, such as an isotopically-labelled biomarker, especially one or more isotopically-labelled internal standards for each biomarker of interest, such as D₇-ceramide (d18:1/18:0), D₇-ceramide (d18:1/22:0), D₇-ceramide (d18:1/24:0), D₇-ceramide (d18:1/24:1), D₇-ceramide (d20:1/24:0), D₇-ceramide (d20:1/24:1), D₉-phosphatidylcholine (16:0/16:0), D₉-sphingomyelin (d18:1/16:0) or D₃₁-sphingomyelin (d18:1/16:0); especially D₇-ceramide (d18:1/18:0), D₇-ceramide (d18:1/24:0), D₇-ceramide (d18:1/24:1), D₉-phosphatidylcholine (16:0/16:0), D₉-sphingomyelin (d18:1/16:0) or D₃₁-sphingomyelin (d18:1/16:0).

Accordingly, in another aspect, there is provided a kit for determining or predicting prognosis or survival for a subject with prostate cancer, comprising one or more reagents for determining the level of at least three lipid biomarkers in a biological sample from the subject, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0).

In the context of the present invention, “kit” is understood to mean a product containing the different reagents necessary for carrying out the methods of the invention packed so as to allow their transport and storage. Optionally, the kits may contain instructions for the simultaneous, sequential or separate use of the different components contained in the kit. The instructions may be in the form of printed material or in the form of an electronic support capable of storing instructions such that they can be read by a subject, such as electronic storage media (e.g. magnetic disks, tapes and the like), optical media (e.g. CD-ROM or DVD) and the like. Alternatively or in addition, the media can contain internet addresses or links thereto that provide the instructions. The kits may contain software for interpreting assay data to determine the prognosis and/or survival of the subject. In some embodiments, the kits may provide a means to access a machine learning system provided, for example, as a software as a service (SaaS) deployment.

In specific embodiments, the one or more reagents comprises a labelled lipid, such as an isotope-labelled lipid, corresponding to each of the at least three lipid biomarkers in the sample. Exemplary isotope-labelled lipids are discussed supra. The reagents may be in liquid form or can be lyophilised or otherwise dried. Suitable containers for the reagents include, for example, bottles, vials, syringes, and test tubes, which may be formed from a variety of materials, including glass or plastic. The kit may also comprise a package insert containing written instructions for the methods described herein (e.g. methods of determining the prognosis or survival for a subject).

In some embodiments, the lipid biomarkers are selected from the group consisting of ceramide (d18:1/18:0), ceramide (d18:1/22:0), ceramide (d18:1/24:0), ceramide (d18:1/24:1), ceramide (d20:1/24:1) and phosphatidylcholine (16:0/16:0); especially wherein the lipid biomarkers are selected from the group consisting of ceramide (d18:1/18:0), ceramide (d18:1/22:0), ceramide (d18:1/24:0), ceramide (d18:1/24:1) and ceramide (d20:1/24:1). In some embodiments, the lipid biomarkers are ceramide (d18:1/18:0), ceramide (d18:1/24:0) and ceramide (d18:1/24:1).

In some embodiments, the kit may further comprise one or more reagents for determining the level of total cholesterol, triglycerides and/or high-density lipoprotein in the biological sample. For example, the one or more reagents may include an enzyme, such as cholesterol esterase or a derivative thereof, cholesterol oxidase or a derivative thereof and/or lipase.

In an alternative aspect, the kit comprises one or more reagents for determining the level of at least three biomarkers in a biological sample from the subject, wherein the biomarkers comprise at least one biomarker selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0); and at least two biomarkers selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0), sphingomyelin (d18:1/16:0), total cholesterol, triglycerides and high-density lipoprotein. In some embodiments, the biomarkers are ceramide (d18:1/18:0), ceramide (d18:1/24:0), ceramide (d18:1/24:1), total cholesterol and triglycerides.

Suitable biological samples are discussed in detail herein. In some embodiments, the biological sample is blood, serum or plasma; especially plasma. The kit may optionally include a container and/or device (such as a syringe) for collection of the biological sample from the subject.

The kit may further comprise appropriate reagents for detection of labels (where appropriate), positive and negative controls, washing solutions, blotting membranes, microtiter plates, dilution buffers and the like. The kit may also comprise reagents for enriching and/or extracting lipid components, representative examples of which include an alcohol (e.g. 1-butanol, methanol, ethanol, isopropanol and the like), and optionally a protein precipitant such as ammonium formate or ammonium sulfate.

### 5. Methods of Treatment

The methods for determining the prognosis and/or survival of a subject are also useful for managing treatment decisions for the subject having prostate cancer, especially metastatic castration-resistant prostate cancer, including treating or inhibiting or delaying the progression of the cancer. For example, where a subject is identified as having a poor prognosis (e.g. disease progression and/or death from disease within about 18 months from commencement of chemotherapy or treatment with an androgen receptor signalling inhibitor) or a likelihood of non-survival using the methods of the invention, the subject may be administered an alternative therapy, including combination therapy, an increased dosage of a cancer therapy, an alternative cancer therapy, or may be placed into palliative care. In some embodiments, a subject identified as having a poor prognosis may be administered an alternative cancer therapy, either alone or in combination with standard or existing therapies, such as a lipid targeted therapy.

Accordingly, in a further aspect, there is provided a method for treating or inhibiting the progression of prostate cancer in a subject, comprising, consisting or consisting essentially of:
a) identifying a subject with prostate cancer having a poor prognosis using the method of the invention; and
b) administering a treatment for the prostate cancer.

Suitable treatments include, but are not limited to, a lipid targeted therapy (i.e. a therapy directly or indirectly targeting one or more lipids, such as lipid activity, lipid metabolism or lipid synthesis), such as a ceramide inhibitor. Suitable lipid targeted therapies include, but are not limited to, a statin, a PCSK9 inhibitor, a sphingosine kinase 1 and/or 2 inhibitor, a Des1 inhibitor and/or a SREBP inhibitor. Suitable statins, PCSK9 inhibitors, sphingosine kinase 1 and/or 2 inhibitors, Des1 inhibitors and/or SREBP inhibitors are discussed in Section 3 supra. In particular embodiments, the subject is administered a PCSK9 inhibitor, such as evolocumab, or a sphingosine kinase 1 and/or 2 inhibitor, such as opaganib.

The treatment may, alternatively or in addition, comprise surgery, radiotherapy, chemotherapy and other cancer therapies.

Radiotherapies include radiation and waves that induce DNA damage for example, γ-irradiation, X-rays, UV irradiation, microwaves, electronic emissions, radioisotopes, and the like. Therapy may be achieved by irradiating the localized tumour site with the above described forms of radiations. It is most likely that all of these factors effect a broad range of damage DNA, the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells. Non-limiting examples of radiotherapies include conformal external beam radiotherapy (50-100 Grey given as fractions over 4-8 weeks), either single shot or fractionated, high dose rate brachytherapy, permanent interstitial brachytherapy or systemic radio-isotopes (e.g. Strontium 89 or Lutetium-177 Prostate Specific Membrane Antigen). In some embodiments the radiotherapy may be administered in combination with a radiosensitising agent, representative examples of which include efaproxiral, etanidazole, fluosol, misonidazole, nimorazole, temoporfin and tirapazamine.

Numerous cancer therapy agents exists including chemotherapeutic agents, which may be cytostatic or cytotoxic, such as antiproliferative/antineoplastic drugs and combinations thereof, as used in medical oncology (e.g. alkylating agents such as cisplatin, carboplatin, cyclophosphamide, nitrogen mustard, melphalan, chlorambucil, busulphan and nitrosoureas; antimetabolites such as antifolates including fluoropyridines like 5-fluorouracil and tegafur, raltitrexed, methotrexate, cytosine arabinoside and hydroxyurea; anti-tumour antibiotics e.g. anthracyclines like adriamycin, bleomycin, doxorubicin, daunomycin, epirubicin, idarubicin, mitomycin-C, dactinomycin and mithramycin; antimitotic agents e.g. vinca alkaloids like vincristine, vinblastine, vindesine and vinorelbine and taxoids like paclitaxel and docetaxel; and topoisomerase inhibitors such as epipodophyllotoxins like etoposide and teniposide, amsacrine, topotecan and camptothecin); cytostatic agents (e.g. such as antiestrogens for example tamoxifen, toremifene, raloxifene, droloxifene and idoxifene; oestrogen receptor down regulators e.g. fulvestrant; antiandrogens such as bicalutamide, flutamide, nilutamide and cyproterone acetate; UH antagonists or LHRH agonists such as goserelin, leuprorelin and buserelin; progestogens such as megestrol acetate; aromatase inhibitors including anastrozole, letrozole, vorozole and exemestane; and inhibitors of 5α-reductase such as finasteride); agents which inhibit cancer cell invasion (e.g. metalloproteinase inhibitors like marimastat and inhibitors of urokinase plasminogen activator receptor function); inhibitors of growth factor function (e.g. growth factor antibodies, growth factor receptor antibodies such as the anti-erbb2 antibody trastuzumab and the anti-erbb1 antibody cetuximab; farnesyl transferase inhibitors, MEK inhibitors, tyrosine kinase inhibitors and serine/threonine kinase inhibitors, other inhibitors of the epidermal growth factor family such as other EGFR family tyrosine kinase inhibitors including N-(3-chloro-4-fluorophenyl)-7-methoxy-6-(3-morpholinopropoxy) quinazolin-4-amine (gefitinib, AZD1839), N-(3-ethynylphenyl)-6,7-bis (2-methoxyethoxy) quinazolin-4-amine (erlotinib, OSI-774) and 6-acrylamido-N-(3-chloro-4-fluorophenyl)-7-(3-morpholinopropoxy) quinazolin-4-amine (CI 1033), inhibitors of the platelet-derived growth factor family and inhibitors of the hepatocyte growth factor family); anti-angiogenic agents (e.g. those which inhibit the effects of vascular endothelial growth factor, for example, the anti-vascular endothelial cell growth factor antibody bevacizumab; compounds such as those disclosed in WO 97/22596, WO 97/30035, WO 97/32856 and WO 98/13354; and compounds that work by other mechanisms such as linomide, inhibitors of integrin αvβ3 function and angiostatin); vascular damaging agents (e.g. Combretastatin A4 and compounds disclosed in WO 99/02166, WO00/40529, WO 00/41669, WO01/92224, WO02/04434 and WO02/08213); antisense therapies (e.g. those which are directed to the targets listed above, such as ISIS 2503, an antisense inhibitor of H-ras); gene therapy approaches (e.g. approaches to replace aberrant genes such as aberrant p53 or aberrant GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy); immunotherapy approaches (e.g. immune checkpoint inhibitors such as those that target CTLA-4 and thus block or inhibit the interaction between CTLA-4 and CD80/CD86 e.g. CTLA-4 inhibitors, such as ipilimumab or tremelimumab; those that target PD-1 and thus block or inhibit the interaction between PD-1 and PD-L1 e.g. PD-1 inhibitors, representative examples of which include pembrolizumab, pidilizumab, nivolumab, REGN2810, CT-001, AMP-224, BMS-936558, MK-3475, MEDI0680 and PDR001; and those that target PD-L1 and thus block or inhibit the interaction between PD-1 and PD-L1 e.g. PD-L1 inhibitors such as atezolizumab, durvalumab, avelumab, BMS-936559 and MEDI4736; and ex vivo and in vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies).

Typically, cancer treatments and therapies, such as a lipid targeted therapy, are administered in pharmaceutical compositions together with a pharmaceutically acceptable carrier and in an effective amount to achieve their intended purpose. The dose of active compounds administered to a subject should be sufficient to achieve a beneficial response in the subject over time, such as a reduction in tumour burden, an improved prognosis, and the like. The quantity of the pharmaceutically active compounds(s) to be administered may depend on the subject to be treated inclusive of the age, sex, weight and general health condition thereof. A skilled person will readily be able to determine the suitable amounts and/or dosages of the active compound(s) for administration and suitable treatment regimens without undue experimentation.

The treatment may be administered in concert with an adjunctive cancer therapy or treatment, representative examples of which include agents to reduce pain, hair loss, vomiting, immune suppression, nausea, diarrhoea, rash, sensory disturbance, anaemia and fatigue.

If a subject is identified as having a good prognosis (e.g. no disease progression and/or no death from disease within about 18 months), for instance after being administered a cancer treatment, the patient may be continued to be administered the cancer treatment or cease to be administered the cancer treatment.

The methods of the invention may also be used to select a subject with prostate cancer, especially metastatic castration-resistant prostate cancer, for treatment with a lipid targeted therapy. For such uses, the methods involve identifying a subject with prostate cancer having a poor prognosis using the method of the invention.

Suitable lipid targeted therapies are as described supra. In some embodiments, the treatment comprises administering a PCSK9 inhibitor, such as evolocumab, or a sphingosine kinase 1 and/or 2 inhibitor, such as opaganib.

The response of a subject with prostate cancer, especially metastatic castration-resistant prostate cancer, to a therapeutic treatment may be monitored using the methods of the invention. Such methods comprise, consist or consist essentially of:
a) determining the level of at least three lipid biomarkers in a first biological sample obtained from the subject, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0), and the first biological sample is obtained before or after commencement of treatment;
b) determining a first indicator using the biomarker levels;
c) determining the level of at least three lipid biomarkers in a second biological sample obtained from the subject, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0), and the second biological sample is obtained at a time point after commencement of therapeutic treatment and after the first biological sample is obtained;
d) determining a second indicator using the biomarker levels; and
e) comparing the indicator in the first and second biological samples;
wherein a change in the indicator between the first and second biological samples is indicative of whether or not the subject is responding to the therapeutic treatment.

Suitable embodiments of the method are as discussed above, including biological samples and methods for determining the biomarker levels and the indicator.

In some embodiments, the lipid biomarkers in steps a) and c) are selected from the group consisting of ceramide (d18:1/18:0), ceramide (d18:1/22:0), ceramide (d18:1/24:0), ceramide (d18:1/24:1), ceramide (d20:1/24:1) and phosphatidylcholine (16:0/16:0); especially wherein the lipid biomarkers in steps a) and c) are selected from the group consisting of ceramide (d18:1/18:0), ceramide (d18:1/22:0), ceramide (d18:1/24:0), ceramide (d18:1/24:1) and ceramide (d20:1/24:1). In particular embodiments, the lipid biomarkers in steps a) and c) are ceramide (d18:1/18:0), ceramide (d18:1/24:0) and ceramide (d18:1/24:1).

The method may comprise, as discussed in detail elsewhere herein, determining the level of one or more additional biomarkers in steps a) and c), such as total cholesterol, triglycerides and/or high-density lipoprotein; especially the level of one or more biomarkers selected from the group consisting of total cholesterol and triglycerides. In specific embodiments, steps a) and c) further comprise determining the level of total cholesterol and triglycerides. In particular embodiments, steps a) and c) comprise determining the level of ceramide (d18:1/18:0), ceramide (d18:1/24:0), ceramide (d18:1/24:1), total cholesterol and triglycerides.

Alternatively, steps a) and c) may comprise determining the level of at least three biomarkers in a biological sample obtained from the subject, wherein the biomarkers comprise at least one biomarker selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0); and at least two biomarkers selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0), sphingomyelin (d18:1/16:0), total cholesterol, triglycerides and high-density lipoprotein. In particular embodiments, step a) comprises determining the level of ceramide (d18:1/18:0), ceramide (d18:1/24:0), ceramide (d18:1/24:1), total cholesterol and triglycerides.

The method may comprise determining the levels of particular combinations of the biomarkers. For example, in some embodiments, the method comprises determining the level of ceramide (d18:1/18:0), ceramide (d18:1/24:0), total cholesterol and triglycerides; ceramide (d18:1/18:0), ceramide (d18:1/24:0) and ceramide (d20:1/24:1); ceramide (d18:1/18:0), ceramide (d18:1/24:1), ceramide (d18:1/22:0), total cholesterol and triglycerides; ceramide (d18:1/18:0), ceramide (d18:1/22:0), ceramide (d18:1/24:0), ceramide (d18:1/24:1), ceramide (d20:1/24:0), ceramide (d20:1/24:1) and total cholesterol; ceramide (d18:1/18:0), ceramide (d18:1/22:0), ceramide (d18:1/24:0), ceramide (d18:1/24:1), ceramide (d20:1/24:0) and ceramide (d20:1/24:1); ceramide (d18:1/18:0), ceramide (d18:1/24:1), phosphatidylcholine (16:0/16:0) and total cholesterol; ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and total cholesterol; or ceramide (d18:1/18:0), ceramide (d18:1/24:0), ceramide (d18:1/24:1) and total cholesterol.

In some embodiments, the first indicator may be determined before administering a therapeutic treatment and the second indicator may be determined after administration of the therapeutic treatment to the subject. A change in indicator from a poor prognosis (e.g. first indicator) to a good prognosis (e.g. second indicator) indicates a likelihood that the therapeutic treatment was effective in treating the cancer and that the cancer is not progressing. If there is no change in indicator and the prognosis remains poor, for example, this indicates a likelihood that the therapeutic treatment was ineffective in treating the cancer and that the cancer is progressing.

In some examples, the time difference between the first sample (i.e. early time point) and the second sample (i.e. later time point) is at least about 1 week to at least about 3 years (and all integer weeks and months therebetween), including at least about 1, 2, 3, 4, 5, 6, 7, 8, 9, 10, 11 or 12 weeks or at least about 1, 2, 3, 4, 5, 6, 7, 8, 9, 10, 11 or 12 months, or at least about 1, 2 or 3 years. The time difference could also be determined by the number of treatment cycles. In some examples, the time difference between the early time point and the later time point is 1, 2, 3, 4, 5, 6, 7, 8, 9, 10, 11 or 12 treatment cycles.

If the indicator has changed to indicate a likelihood of a poor prognosis, the subject may be administered a further or more aggressive treatment for prostate cancer, such as a lipid targeted therapy. Suitable treatments are as discussed supra. Alternatively, if the indicator has changed to indicate a likelihood of a good prognosis, the current treatment regime for the subject may be maintained and/or ceased (e.g. if the subject is being administered a lipid targeted therapy).

### 6. Devices

Further encompassed are devices and apparatuses for implementing the methods of the invention, such as a processing device. Accordingly, in another aspect, there is provided an apparatus for determining an indicator used in assessing prognosis for a subject with prostate cancer, the apparatus comprising at least one electronic processing device that:
a) determines a biomarker level of at least three lipid biomarkers in a biological sample obtained from the subject, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0); and
b) determines the indicator using the derived biomarker levels.

Suitable embodiments of the prognostic method are discussed in Section 3 supra, including the type of prostate cancer, the biological sample, preferred biomarkers and combinations thereof, and methods for determining the biomarker levels and indicator.

In preferred embodiments, the lipid biomarkers are selected from the group consisting of ceramide (d18:1/18:0), ceramide (d18:1/22:0), ceramide (d18:1/24:0), ceramide (d18:1/24:1), ceramide (d20:1/24:1) and phosphatidylcholine (16:0/16:0); especially the group consisting of ceramide (d18:1/18:0), ceramide (d18:1/22:0), ceramide (d18:1/24:0), ceramide (d18:1/24:1) and ceramide (d20:1/24:1). In particular embodiments, the lipid biomarkers are ceramide (d18:1/18:0), ceramide (d18:1/24:0) and ceramide (d18:1/24:1).

Step a) may further comprise determining the level of one or more biomarkers selected from the group consisting of total cholesterol, triglycerides and high-density lipoprotein; especially one or more biomarkers selected from the group consisting of total cholesterol and triglycerides. In specific embodiments, step a) further comprises determining the level of total cholesterol and triglycerides. In particular embodiments, step a) comprises determining the level of ceramide (d18:1/18:0), ceramide (d18:1/24:0), ceramide (d18:1/24:1), total cholesterol and triglycerides.

Step a) may, alternatively, comprise determining the level of at least three biomarkers in a biological sample obtained from the subject, wherein the biomarkers comprise at least one biomarker selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0); and at least two biomarkers selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0), sphingomyelin (d18:1/16:0), total cholesterol, triglycerides and high-density lipoprotein. In particular embodiments, step a) comprises determining the level of ceramide (d18:1/18:0), ceramide (d18:1/24:0), ceramide (d18:1/24:1), total cholesterol and triglycerides.

In particular embodiments, the method comprises determining the level of ceramide (d18:1/18:0), ceramide (d18:1/24:0), total cholesterol and triglycerides; ceramide (d18:1/18:0), ceramide (d18:1/24:0) and ceramide (d20:1/24:1); ceramide (d18:1/18:0), ceramide (d18:1/24:1), ceramide (d18:1/22:0), total cholesterol and triglycerides; ceramide (d18:1/18:0), ceramide (d18:1/22:0), ceramide (d18:1/24:0), ceramide (d18:1/24:1), ceramide (d20:1/24:0), ceramide (d20:1/24:1) and total cholesterol; ceramide (d18:1/18:0), ceramide (d18:1/22:0), ceramide (d18:1/24:0), ceramide (d18:1/24:1), ceramide (d20:1/24:0) and ceramide (d20:1/24:1); ceramide (d18:1/18:0), ceramide (d18:1/24:1), phosphatidylcholine (16:0/16:0) and total cholesterol; ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and total cholesterol; or ceramide (d18:1/18:0), ceramide (d18:1/24:0), ceramide (d18:1/24:1) and total cholesterol.

The method may further comprise retrieving previously determined indicator references from a database, the indicator references being determined based on indicators determined from a reference population; comparing the indicator to the indicator references to thereby determine a probability indicative of the subject having or not having a disclosed prognosis; and generating a representation of the probability, the representation being displayed to a user to allow the user to assess the likelihood of the subject having disclosed prognosis.

The apparatus may further include any one or more of a sampling device that obtains a biological sample taken from a subject, the sample including at least three biomarkers as disclosed herein; a measuring device that quantifies the level of each of the biomarkers; and at least one processing device that receives the biomarker levels from the measuring device, determines an indicator that is indicative of a disclosed prognosis using the biomarker levels optionally in combination with one or more clinical parameters or signs of the subject, compares the indicator to at least one indicator reference, determines a likelihood of the subject having or not having the disclosed prognosis using the results of the comparison, and generates a representation of the indicator and the likelihood for display to a user.

In some embodiments, the apparatus comprises a processor configured to execute computer readable media instructions, such as a computer program or software application (e.g. a machine learning system), to receive the biomarker levels from the evaluation of the biomarkers in the biological sample and, in combination with other risk factors (e.g. medical history of the patient, publically available sources of information pertaining to a poor prognosis for prostate cancer) may determine a master composite score and compare it to a grouping of stratified cohort population comprising multiple risk categories (e.g. a risk categorisation table) and provide a risk score. Methods and techniques for determining a master composite score and a risk score are known in the art.

The apparatus can take any of a variety of forms, for example, a handheld device, a tablet, or any other type of computer or electronic device. The apparatus may also comprise a processor configured to execute instructions (e.g. a computer software product), an application for a handheld device, a handheld device configured to perform the method, a world-wide-web (WWW) page or other cloud or network accessible location, or any computing device. In other embodiments, the apparatus may include a handheld device, a tablet or any other type of computer or electronic device for accessing a machine learning system provided as a software as a service (Saas) deployment. Accordingly, the correlation may be displayed as a graphical representation, which, in some embodiments, is stored in a database or memory, such as a random access memory, read-only memory, disk, virtual memory, etc. Other suitable representations, or exemplifications known in the art may also be used.

The apparatus may further comprise a storage means for storing the correlation, an input means, and a display means for displaying the status of the subject in terms of the particular prognosis disclosed herein (e.g. a poor prognosis such as disease progression and/or death from disease within about 18 months from commencement of chemotherapy or treatment with an androgen receptor signalling inhibitor). The storage means can be, for example, random access memory, read-only memory, a cache, a buffer, a disk, virtual memory, or a database. The input means can be, for example, a keypad, a keyboard, stored data, a touch screen, a voice-activated system, a downloadable program, downloadable data, a digital interface, a hand-held device or an infrared signal device. The display means can be, for example, a computer monitor, a cathode ray tube (CRT), a digital screen, a light-emitting diode (LED), a liquid crystal display (LCD), an X-ray, a compressed digitised image, a video image or a hand-held device. The apparatus can further comprise or communicate with a database, wherein the database stores the correlation of factors and is accessible to the user.

In particular embodiments, the apparatus is a computing device, for example, in the form of a computer or hand-held device that includes a processing unit, memory and storage. The computing device can include or have access to a computing environment that comprises a variety of computer-readable media, such as volatile memory and non-volatile memory, removable storage and/or non-removable storage. Computer storage includes, for example, RAM, ROM, EPROM and EEPROM, flash memory or other memory technologies, CD ROM, Digital Versatile Disks (DVD) or other optical disk storage, magnetic cassettes, magnetic tape, magnetic disk storage or other magnetic storage devices, or other medium known in the art to be capable of storing computer-readable instructions. The computing device can also include or have access to a computing environment that comprises input, output and/or a communication connection. The input can be one or several devices, such as a keyboard, mouse, touch screen or stylus. The output can also be one or several devices, such as a video display, a printer, an audio output device, a touch stimulation output device or a screen reading output device. If desired, the computing device can be configured to operate in a networked environment using a communication connection to connect to one or more remote computers. The communication connection can be, for example, a Local Area Network (LAN), a Wide Area Network (WAN) or other networks and can operate over the cloud, a wired network, wireless radio frequency network and/or an infrared network.

In order that the invention may be readily understood and put into practical effect, particular preferred embodiments will now be described by way of the following non-limiting examples.

### EXAMPLES

### Materials and Methods

### Chemicals and Reagents

Standards and deuterated standards of ceramides, phosphatidylcholine and sphingomyelin were purchased from Cayman Chemicals (Ann Arbor, MI, USA) except for D₃₁-SM(d18:1/16:0) which was from BOC sciences (Shirley, NY, USA). Quality control standards (requiring an independent source) for Cer(d18:1/18:0), Cer(d18:1/22:0), Cer(d18:1/24:0) and Cer(d18:1/24:1) were purchased from Toronto Research Chemicals (TRC) (Toronto, ON, Canada), and for PC(16:0/16:0) from Avanti Lipids (Birmingham, AL, USA). A quality control standard for SM(d18:1/16:0) was not available.

All high performance liquid chromatography (HPLC) solvents were LC-MS grade. Acetonitrile, isopropanol, methanol and 1-butanol were sourced from LiChrosolv, Merck (Darmstadt, Germany). Ammonium formate was sourced from Sigma-Aldrich (St Louis, MO, USA). Ultrapure water was produced using the Puris-esse water purification system (Model: Esse-Up Analy-TOC-M, Rotek Australia, Bayswater, Australia).

Cer(d20:1/24:0) and Cer(d20:1/24:1) were synthesised in-house by amide coupling reaction between long chain acids and sphingosines using hydroxybenzotriazole activated esters. All chemicals for the synthesis of cer(d20:1/24:0) and cer(d20:1/24:1) were purchased from Cayman Chemicals except for 1-hydroxybenzotriazole hydrate which was purchased from Toronto Research Chemicals and triethylamine and 1-Ethyl-3-(3-dimethylaminopropyl) carbodiimide) which were purchased from Sigma-Aldrich.

Ceramide (d20:1/24:0) was prepared by adding a solution of triethylamine (15 μmol) and d(20:1) sphingosine (3.3 mg, 10 μmol) in anhydrous dichloromethane (10 mL) dropwise to a solution of lignoceric acid (3.6 mg, 10 μmol), 1-hydroxybenzotriazole hydrate (2.0 mg, 15 μmol) and triethylamine (15 μmol) in anhydrous dichloromethane/acetonitrile (9:1) over 2 minutes. The mixture was stirred for 3 hours before being quenched with aqueous NaHCO₃ (5%, 20 mL). The aqueous layer was discarded, and the organic layer washed with aqueous HCl (0.1 M, 20 mL) and water (20 mL) before being evaporated to dryness. The crude mixture was then redissolved in methanol and purified by HPLC, to yield 1.42 mg of pure ceramide (d20:1/24:0). M/z MRM pairs chosen were 678.6/292.2, 678.6/292.2 and 678.6/280.35 and HPLC retention time was 7.3 minutes.

Ceramide (d20:1/24:1) was synthesised by adding a solution of triethylamine (15 μmol) and d(20:1) sphingosine (3.3 mg, 10 μmol) in anhydrous dichloromethane (10 mL) dropwise to a solution of nervonic acid (3.6 mg, 10 μmol), 1-hydroxybenzotriazole hydrate (2.0 mg, 15 μmol) and triethylamine (15 μmol) in anhydrous dichloromethane/acetonitrile (9:1) over 2 minutes. The mixture was stirred for 3 hours before being quenched with aqueous NaHCO₃ (5%, 20 mL). The aqueous layer was discarded, and the organic layer washed with aqueous HCl (0.1 M, 20 mL) and water (20 mL) before being evaporated to dryness. The crude mixture was then redissolved in methanol and purified by HPLC, to yield 4.90 mg of pure ceramide (d20:1/24:1). M/z MRM pairs chosen were 676.6/292.35, 676.6/280.3 and 676.6/310.2 and HPLC retention time was 6.7 minutes.

### Plasma Samples

A single source of thawed fresh frozen plasma (FFP) (FFP-1) was obtained from the Royal Prince Alfred Blood Bank. This was used for method optimisation, creation of standard curves and quality control (QC) samples.

Clinical plasma samples were collected from two cohorts (Discovery & Validation) prior to starting treatment according to a standardised blood collection protocol (data supplement). The Discovery cohort comprised 105 men with metastatic castration-resistant prostate cancer (mCRPC) commencing taxane chemotherapy at seven sites in New South Wales, Australia. The Validation cohort comprised 183 men with mCRPC commencing taxane chemotherapy (docetaxel or cabazitaxel) or an ARSI (abiraterone or enzalutamide) as first or second line treatment at seven sites in New South Wales and Victoria, Australia.

All participants provided written informed consent (Monash Health Institutional Review Board (15571X), Royal Prince Alfred Hospital Human Research Ethics Committee (X14-0406, X19-0320), Australia-New Zealand Clinical Trials Registry ACTRN12607000077460, ACTRN12611000540910).

For blood collection of the discovery cohort, non-fasted blood samples were obtained from patients. In brief, peripheral blood samples were collected in BD Vacutainer tubes containing K2EDTA, centrifuged at 3000×g for 5 minutes at room temperature, and the plasma aliquoted into cryovials and stored at −80° C. cryostorage until required. For the validation cohort, non-fasted blood samples were obtained from patients. Peripheral blood from patients was sampled and lipid extraction performed as previously described (refer to Lin et al. (2017) International Journal of Cancer, 141:2112-2120; Lin et al. (2021) Prostate Cancer and Prostatic Diseases, 24:860-870; and Lin et al. (2021) EBioMedicine, 72:103625). Whole blood was collected into 10 mL EDTA-containing tubes and two-step centrifugation was performed to separate plasma and buffy coat. Blood was first centrifuged at 1600×g for 15 minutes and the supernatant was transferred to a fresh tube, where it was centrifuged again at 5000×g for 10 minutes. Aliquots of the plasma after the second centrifugation were stored at −80° C. until required.

### Preparation of Stock and Working Solutions

Stock solutions of standards (1 mg/mL) were prepared in isopropanol. A working solution was prepared by diluting stock solutions with 1-butanol/methanol (v/v 1:1) (BuMe) to concentrations of 0.25 mg/L Cer(d18:1/18:0), 3.75 mg/L Cer(d18:1/22:0), 6.5 mg/L Cer(d18:1/24:0), 5.25 mg/L Cer(d18:1/24:1), 0.1 mg/L Cer(d20:1/24:0, 0.1 mg/L Cer(d20:1/24:1), 20 mg/L PC(16:0/16:0) and 50 mg/L SM(d18:1/16:0).

A mixed solution of internal standards (IS) was combined with a protein precipitation solution (PPS) consisting of 10 mM ammonium formate in BuMe. The concentration of the internal standards in PPS were 0.5 mg/L D7-Cer(d18:1/18:0), 0.65 mg/L D7-Cer(d18:1/24:0), 0.52 mg/L D7-Cer(d18:1/24:1), 2.5 mg/L D9-PC(16:0/16:0) and 2.5 mg/L D9-SM(d18:1/16:0) or D31-SM(d18:1/16:0)).

A single source of thawed fresh frozen plasma (FFP) (FFP-1) was obtained from the Royal Prince Alfred Blood Bank. This was used for method optimisation, creation of standard curves and quality control (QC) samples.

### Lipid Extraction of Plasma Samples

800 μL of IS-PPS was combined with 100 μL of plasma and 100 μL of BuME, vortexed for 10 seconds, then sonicated for 60 mins in a water bath at 20° C. and finally centrifuged at 16,000 g for 10 minutes. 750 μL of the supernatant was transferred to a glass vial for LC-MS analysis.

### LC-MS Analysis

Lipid extracts were analysed on a Shimadzu LCMS-8050 using multiple-reaction monitoring (MRM). Optimal mass spectrometer conditions for the analytes were determined by infusing individual lipid standards at 1 μg/mL.

### Determination of Plasma Lipid Concentration

The concentration of lipids in clinical plasma samples were normalised to internal standards (ISs) and calculated from calibration standards consisting of spiked FFP-1 that were freshly prepared and included in every analytical run (refer to Table 1). Spiked FFP-1 were prepared by mixing 100 μL of FFP-1 with 100 μL of lipid standard and extracted in the same manner as the clinical plasma samples (replacing BuMe and plasma with 200 μL of spiked FFP-1). The highest spike concentration was the same as the working solution of the lipid in pure BuMe. Subsequent spike concentrations were respectively 80%, 60%, 40%, 20% and 0% of the working solution. The concentration of the analytes in the calibration standards was pre-determined by the method of standard addition which was performed on eight replicate sets of the calibration standards. For each replicate set, a calibration curve was obtained by simple linear regression of the standards, and the concentration of analytes in blank FFP-1 (blank spike) was extrapolated from the regression line. The mean concentration in blank FFP-1 across the eight replicates was used to calculate the concentration of the analytes at each point on the calibration curve.

Discovery cohort samples were extracted and analysed across two runs. Validation cohort samples were extracted and analysed in 7 runs over 6 days. Each run included FFP-1 calibration standards and QCs. All patients from the Discovery and Validation cohorts had previously been assayed for the presence or absence of the poor prognostic signature reported previously (refer to Lin et al. (2017) International Journal of Cancer, 141:2112-2120; and Lin et al. (2021) EBioMedicine, 72:103625).

### Quality Controls (QC)

QCs, consisting of spiked FFP-1 at three concentrations for each analyte, are included in every analytical run to monitor LC-MS method performance. To minimise error, independent sources of base materials should be used for QCs and calibration standards, thus the analytes for the QCs were sourced from an alternate company to the lipids used in our calibration curves where available. QC concentrations were based on the distribution of concentrations detected in plasma samples in our previously published studies (refer to Lin et al. (2017) International Journal of Cancer, 141:2112-2120; Lin et al. (2021) Prostate Cancer and Prostatic Diseases, 24:860-870; and Lin et al. (2021) EBioMedicine, 72:103625).

### Recovery of Lipids and Matrix Effects

FFP and aqueous calibration standards were analysed in eight replicate runs for linearity of response of analytes. Recovery of the analytes was calculated as the calibration curve slope in FFP divided by the curve slope in water on the same analytical run. Reduction in recovery of <10% was considered non-significant. Matrix effects were determined by comparing analyte concentrations between spikes added prior and following lipid extraction of three different thawed FFP samples, with differences of <10% considered non-significant. The highest calibration spike concentration was used. For pre-extraction spikes, the spike was added to the FFP before lipid extraction. For post-extraction spikes, the spike was added after the sonication step of the lipid extraction process, prior to the centrifugation step.

### Optimal LC-MS Conditions

Optimal LC-MS conditions were as follows: interface temperature, 300° C.; nebulising gas flow, 1.6 L/min; drying gas flow, 10 L/min; heating gas flow, 10 L/min; column oven temperature, 60° C.; ion spray voltage, 4.0 kV. Chromatographic separation was performed on an ACE Excel 2 C18, 50 mm column (Advanced Chromatography Technologies, Ltd, Reading, United Kingdom). Mobile phase consisted of (A) 50% water, 30% acetonitrile, 20% isopropanol with 10 mM ammonium formate and (B) 1% water, 9% acetonitrile, 90% isopropanol with 10 mM ammonium formate. Injection volume was 5 μL and the flow rate was set to 0.8 mL/min. The gradient was as follows: starting with 10% B, increasing to 45% B over 1.9 minutes, then to 53% over 0.01 minutes, to 65% over 4.39 minutes, to 89% over 0.1 minutes, to 92% over 1.3 minutes and to 100% over 0.1 minutes. The solvent was then held at 100% B for 0.5 minutes. Solvent was decreased from 100% B to 10% B over 0.1 minutes and held for an additional 2.1 minutes (for a total cycle time of 10.5 minutes). Dwell time was set at 24 ms for each of the signal transitions except for the transitions belonging to PC(16:0/16:0), SM(d18:1/16:0) and their corresponding internal standards, where the dwell times were set at 3 ms.

M/z MRM pairs and internal standards are provided in Table 1.

TABLE 1
M/z MRM PAIRS AND INTERNAL STANDARDS

| | | Corresponding | Retention |
|---|---|---|---|
| | m/z MRM | deuterated | time |
| Lipid species | pair | internal standard | (mins) |
| Cer(d18:1/18:0) | 566.5/264.3 | D₇-Cer(d18:1/18:0) | 4.6 |
| | 566.5/282.3 | | |
| Cer(d18:1/22:0) | 622.6/264.2 | D₇-Cer(d18:1/24:1) | 5.95 |
| | 622.6/282.2 | | |
| Cer(d18:1/24:0) | 650.6/264.3 | D₇-Cer(d18:1/24:0) | 6.7 |
| | 650.6/282.3 | | |
| Cer(d18:1/24:1) | 648.6/264.3 | D₇-Cer(d18:1/24:1) | 5.9 |
| | 648.6/282.3 | | |
| Cer(d20:1/24:0) | 678.6/292.2 | D₇-Cer(d18:1/24:0) | 7.3 |
| | 678.6/292.2 | | |
| | 678.6/280.35 | | |
| Cer(d20:1/24:1) | 676.6/292.35 | D₇-Cer(d18:1/24:0) | 6.7 |
| | 676.6/280.3 | | |
| | 676.6/310.2 | | |
| PC (16:0/16:0) | 734.5/184.55 | D₉-PC(16:0/16:0) | 4.3 |
| | 734.5/478.6 | | |
| | 734.5/496.7 | | |
| SM(d18:1/16:0) | 704.5/185.1 | D₉-SM(d18:1/16:0) or | 3.6 |
| | 704.5/126.0 | D₃₁-SM(d18:1/16:0) | |
| D₇-Cer(d18:1/18:0) | 573.5/259.3 | — | 4.6 |
| | 573.5/289.3 | | |
| D₇-Cer(d18:1/24:0) | 657.6/271.3 | — | 6.7 |
| | 657.6/289.3 | | |
| D₇-Cer(d18:1/24:1) | 655.6/271.3 | — | 5.9 |
| | 655.6/289.3 | | |
| D₉-PC(16:0/16:0) | 744.5/185.2 | — | 4.3 |
| | 744.5/488.3 | | |
| | 744.5/506.5 | | |
| D₉-SM(d18:1/16:0) | 713.6/185.1 | — | 3.6 |
| | 713.6/125.7 | | |
| D₃₁-SM(d18:1/16:0) | 736.6/185.15 | — | 3.7 |
| | 736.6/126.0 | | |

Abbreviations: m/z, mass to charge ratio; MRM, multiple reaction monitoring; Cer, Ceramide; PC, phosphatidylcholine; SM, sphingomyelin.

Due to the several-fold higher concentrations of PC(16:0/16:0) and SM(d18:1/16:0) in endogenous plasma, the m/z MRM pairs for PC(16:0/16:0) and SM(d18:1/16:0) were adjusted in order to attenuate the signal. This involved increasing the m/z of the product ions for PC(16:0/16:0) by 0.2 and the SM(d18:1/16:0) parent and product ions by 1.0 to give the final m/z MRM pairs described above. The optimised collision energies were also adjusted by +10. The corresponding ISs were adjusted accordingly.

No signals of the respective ISs were observed in extracted blank FFP (when extracted without the presence of ISs), showing no interference coming from the IS. The retention time of respective endogenous species matched the ISs. D₇-cer(d18:1/24:1) was used as the IS for both Cer(d18:1/22:0) and Cer(d18:1/24:1), and D₇-Cer(d18:1/24:0) was used as the IS for Cer(d18:1/24:0), Cer(d20:1/24:0) and Cer(d20:1/24:1) due to their similar retention times and the unavailability of a deuterated standard for Cer(d18:1/22:0), Cer(d20:1/24:0) and Cer(d20:1/24:1).

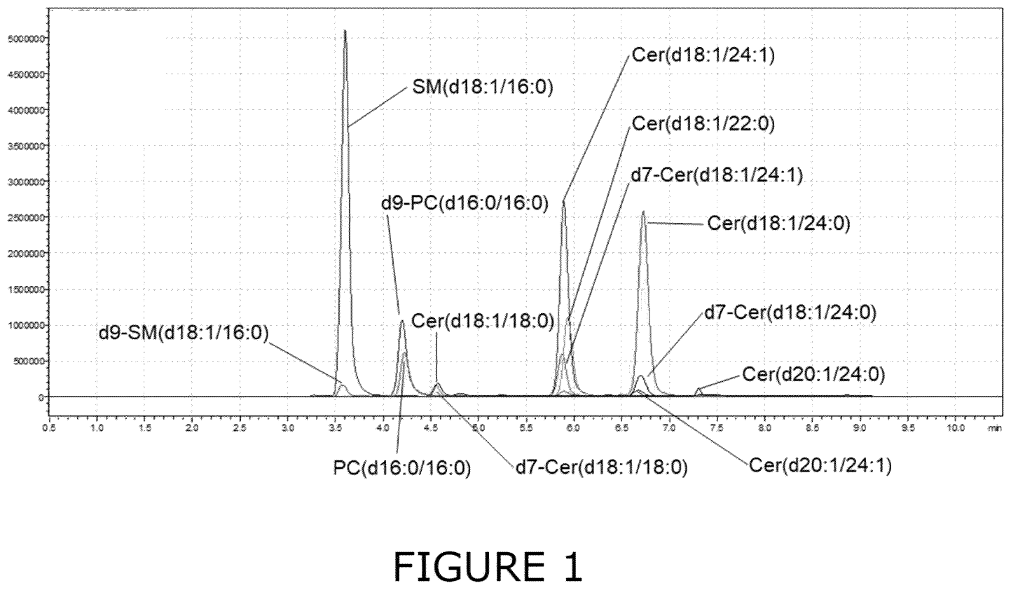

The LC-MS approach provided baseline separation of all lipid species (refer to FIG. 1).

### Linearity of Calibration Curves, Analyte Recovery and Matrix Effects

The calibration curves of lipid standards prepared in plasma showed linearity for all candidate lipids with mean coefficients of determination of 0.938-0.998 (refer to Table 2). The recovery of the spiked lipid standards from plasma ranged from 93-102% for all lipids except for SM(d18:1/16:0) at 44% and cer(d20:1/24:0) at 72% (refer to Table 3). In order to include a wide variety of analytes in a single assay, these two low recovery values were considered acceptable. Plasma matrix effects were minimal, with pre-extraction peak areas being 97-115% of the post extraction peak areas across all lipid species (refer to Table 3).

TABLE 2
RESULTS OF LINEARITY ASSESSMENT FOR EACH LIPID IN FFP
ACROSS EACH RUN REPLICATE FOR THE DETERMINATION OF
THE CONCENTRATIONS ON THE CALIBRATION CURVE
Coefficient of Determination (R²)

| | Cer | Cer | Cer | Cer |
|---|---|---|---|---|
| Run no: | (d18:1/18:0) | (d18:1/22:0) | (d18:1/24:0) | (d18:1/24:1) |
| 1 | 0.9642 | 0.994 | 0.9918 | 0.9981 |
| 2 | 0.9937 | 0.9979 | 0.9864 | 0.9997 |
| 3 | 0.9937 | 0.9953 | 0.9947 | 0.9891 |
| 4 | 0.9856 | 0.9986 | 0.9763 | 0.999 |
| 5 | 0.9714 | 0.999 | 0.9962 | 0.9992 |
| 6 | 0.9739 | 0.9949 | 0.9875 | 0.9979 |
| 7 | 0.9628 | 0.999 | 0.9889 | 0.9989 |
| 8 | 0.9424 | 0.99 | 0.9396 | 0.9982 |
| Mean | 0.973 | 0.996 | 0.983 | 0.998 |
| % CV | 1.68 | 0.30 | 1.76 | 0.32 |
| | Cer | Cer | PC | SM |
| Run no: | (d20:1/24:0) | (d20:1/24:1) | (16:0/16:0) | (d18:1/16:0) |
| 1 | 0.9987 | 0.9984 | 0.9923 | 0.9831 |
| 2 | 0.9879 | 0.9926 | 0.9906 | 0.9763 |
| 3 | 0.995 | 0.9982 | 0.9768 | 0.9897 |
| 4 | 0.9897 | 0.994 | 0.9938 | 0.9686 |
| 5 | 0.9771 | 0.9965 | 0.993 | 0.9111 |
| 6 | 0.987 | 0.9844 | 0.9457 | 0.9235 |
| 7 | 0.9952 | 0.9987 | 0.9344 | 0.8671 |
| 8 | 0.9949 | 0.9988 | 0.9922 | 0.8872 |
| Mean | 0.991 | 0.995 | 0.977 | 0.938 |
| % CV | 0.65 | 0.46 | 2.28 | 4.71 |

Abbreviations: Cer, Ceramide; PC, phosphatidylcholine; SM, sphingomyelin.

TABLE 3
ASSAY RECOVERY AND PLASMA MATRIX EFFECTS

| Analyte | | | |
|---|---|---|---|
| cer | cer | cer | cer |
| (d18:1/18:0) | (d18:1/22:0) | (d18:1/24:0) | (d18:1/24:1) |

Assay recovery (%)

| | | | | |
|---|---|---|---|---|
| Mean | 93.92 | 96.48 | 101.97 | 101.08 |
| % CV | 5.74 | 2.88 | 6.73 | 5.59 |

Matrix effects (%)

| Mean | 97.47 | 106.72 | 108.91 | 106.60 |
|---|---|---|---|---|
| % CV | 1.01 | 1.84 | 1.52 | 2.69 |
| | Analyte | | | |
| | cer | cer | PC | SM |
| | (d20:1/24:0) | (d20:1/24:1) | (16:0/16:0) | (d18:1/16:0) |

Assay recovery (%)

| | | | | |
|---|---|---|---|---|
| Mean | 72.39 | 102.00 | 93.75 | 44.00 |
| % CV | 5.18 | 8.43 | 19.33 | 15.63 |

Matrix effects (%)

| | | | | |
|---|---|---|---|---|
| Mean | 100.25 | 114.09 | 98.72 | 99.01 |
| % CV | 3.20 | 6.02 | 1.22 | 4.27 |

Abbreviations: Cer, Ceramide; PC, phosphatidylcholine, SM, sphingomyelin, % CV, coefficient of variation.

### Quality Control (QC) Samples and Stability Assessments

QCs were prepared fresh for every analytical run according to the spike concentrations in Table 4. The spiked concentrations of low-quality control (LQC), medium-quality control (MQC) and high-quality control (HQC) were respectively based on the 20^{th}, 50^{th} and 80^{th} percentiles of endogenous plasma concentrations measured by previous studies (refer to (refer to Lin et al. (2017) International Journal of Cancer, 141: 2112-2120; Lin et al. (2021) Prostate Cancer and Prostatic Diseases, 24:860-870; and Lin et al. (2021) EBioMedicine, 72:103625). The spiked concentration for upper limit of quantification quality control (ULOQ) was double the 80th percentile value. The lower limit of quantification quality control (LLOQ) was prepared using a stock solution made at a concentration of 10% of Standard 2 of the calibration spike. Samples were extracted according to usual sample preparation; however, an aqueous solution of 1% bovine serum albumin was used instead of FFP-1.

TABLE 4
CONCENTRATION OF SPIKED ANALYTES
IN QUALITY CONTROLS

| | | | | Upper Limit of |
|---|---|---|---|---|
| | Low | Medium | High | Quantification |
| Lipid species | (mg/L) | (mg/L) | (mg/L) | (mg/L) |
| Cer (d18:1/18:0) | 0.05 | 0.064 | 0.074 | 0.149 |
| Cer (d18:1/22:0) | 0.714 | 0.857 | 1.429 | 2.857 |
| Cer (d18:1/24:0) | 1.857 | 2.429 | 2.857 | 5.714 |
| Cer (d18:1/24:1) | 0.857 | 1.143 | 1.429 | 2.857 |
| Cer (d20:1/24:0) | 0.017 | 0.026 | 0.029 | 0.057 |
| Cer (d20:1/24:1) | 0.011 | 0.014 | 0.02 | 0.04 |
| PC (16:0/16:0) | 4.857 | 9.429 | 13.429 | 26.857 |
| SM (d18:1/16:0) | 67.143 | 80 | 92.857 | 185.714 |

Abbreviations: Cer, Ceramide; PC, phosphatidylcholine, SM, sphingomyelin.

On-board and in-freezer stability were determined by analysis of freshly extracted QC samples compared to the same samples re-analysed after being stored in the autosampler for 96 hours and in the −20° C. freezer for seven days respectively. Mean recovery for on-board stability testing was between 95-104% for all species except Cer(d20:1/24:0) where it was 80% (refer to Table 5) and mean recovery for in-freezer stability testing was between 93-120% for all species (refer to Table 6).

TABLE 5
ON BOARD STABILITY - MEAN RECOVERY OF
ANALYTES FROM LQC, MQC, HQC AND ULOQ

| | Analyte | | | |
|---|---|---|---|---|
| | Cer | Cer | Cer | Cer |
| | (d18:1/18:0) | (d18:1/22:0) | (d18:1/24:0) | (d18:1/24:1) |
| Mean | 103.48 | 103.48 | 101.43 | 100.56 |
| recovery | | | | |
| (%) | | | | |
| Standard | 3.67 | 2.92 | 7.30 | 2.19 |
| deviation | | | | |
| % CV | 2.82 | 2.82 | 7.20 | 2.17 |
| | Analyte | | | |
| | Cer | Cer | PC | SM |
| | (d20:1/24:0) | (d20:1/24:1) | (16:0/16:0) | (d18:1/16:0) |
| Mean | 79.61 | 100.83 | 95.42 | 96.45 |
| recovery | | | | |
| (%) | | | | |
| Standard | 8.99 | 5.08 | 13.83 | 7.62 |
| deviation | | | | |
| % CV | 11.29 | 5.03 | 14.49 | 7.90 |

Abbreviations: Cer, Ceramide; PC, phosphatidylcholine; SM, sphingomyelin; % CV, coefficient of variation.

TABLE 6
IN-FREEZER STABILITY - MEAN RECOVERY OF
ANALYTES FROM LQC, MQC, HQC AND ULOQ

| | Analyte | | | |
|---|---|---|---|---|
| | Cer | Cer | Cer | Cer |
| | (d18:1/18:0) | (d18:1/22:0) | (d18:1/24:0) | (d18:1/24:1) |
| Mean | 102.67 | 98.91 | 94.97 | 98.54 |
| recovery | | | | |
| (%) | | | | |
| Standard | 8.88 | 8.33 | 6.79 | 7.44 |
| deviation | | | | |
| % CV | 8.65 | 8.42 | 7.15 | 7.55 |
| | Analyte | | | |
| | Cer | Cer | PC | SM |
| | (d20:1/24:0) | (d20:1/24:1) | (16:0/16:0) | (d18:1/16:0) |
| Mean | 114.21 | 119.30 | 100.56 | 93.51 |
| recovery | | | | |
| (%) | | | | |
| Standard | 11.16 | 16.30 | 8.18 | 4.35 |
| deviation | | | | |
| % CV | 9.78 | 13.67 | 8.14 | 4.65 |

Abbreviations: Cer, Ceramide; PC, phosphatidylcholine; SM, sphingomyelin; % CV, coefficient of variation.

Stability across three freeze/thaw (F/T) cycles was determined in five human plasma samples. Plasma had been stored for at least five years in a −80° C. freezer and had been collected as part of the same trial protocol as the Discovery cohort. This plasma was thawed and 100 μL of plasma was extracted according to the standard protocol, and analysed on the LC-MS. The remaining plasma was refrozen at −80° C. This process was repeated twice over the subsequent two weeks. Recovery was calculated as the percentage of the concentration in the first freeze/thawed sample of each of the lipid species in each plasma sample. Mean recovery was calculated using the recovery of each freeze thaw cycle, on each of the plasma samples. Mean recovery was >97% for each analyte (refer to Table 7).

TABLE 7
MEAN RECOVERY OF ANALYTES FROM PLASMA
SAMPLES ACROSS THREE FREEZE-THAW CYCLES

| | Analyte | | | |
|---|---|---|---|---|
| | Cer | Cer | Cer | Cer |
| | (d18:1/18:0) | (d18:1/22:0) | (d18:1/24:0) | (d18:1/24:1) |
| Mean | 99.53 | 96.67 | 111.19 | 97.74 |
| recovery | | | | |
| (%) | | | | |
| Standard | 4.45 | 4.68 | 11.55 | 8.57 |
| deviation | | | | |
| % CV | 4.47 | 4.85 | 10.39 | 8.77 |
| | Analyte | | | |
| | Cer | Cer | PC | SM |
| | (d20:1/24:0) | (d20:1/24:1) | (16:0/16:0) | (d18:1/16:0) |
| Mean | 104.74 | 102.97 | 105.62 | 115.33 |
| recovery | | | | |
| (%) | | | | |
| Standard | 13.18 | 4.23 | 15.59 | 19.27 |
| deviation | | | | |
| % CV | 12.58 | 4.11 | 14.76 | 16.71 |

Abbreviations: Cer, Ceramide; PC, phosphatidylcholine; SM, sphingomyelin; % CV, coefficient of variation.

### Inter- and Intra-Assay Variation

Inter-assay variation was determined by analysing precision (denoted by percentage coefficient of variation (% CV) of each analyte at the LQC, MQC and HQC over eight run replicates (refer to Table 8). Intra-assay variation was determined by analysing seven replicates of LQC, MQC and HQC in the same analytical run (refer to Table 9). Target % CV was below ten percent. CVs were below ten percent for all lipids except Cer(d20:1/24:0) and Cer(d20:1/24:1) where it was <13%. The higher CV of these two lipids reflects their lower endogenous concentration and remains acceptable as the variables are continuous and used for prognostication.

TABLE 8
INTER-ASSAY VARIABILITY
OF ANALYTES IN QC SAMPLES

| | | Mean | | Expected | |
|---|---|---|---|---|---|
| | | concen- | | concen- | |
| | QC | tration | | tration | Accuracy |
| Analyte | sample | (mg/L) | % CV | (mg/L) | (%) |
| Cer(d18:1/18:0) | LQC | 0.08 | 3.9 | 0.08 | 105.94 |
| | MQC | 0.11 | 1.8 | 0.094 | 115.96 |
| | HQC | 0.12 | 1.7 | 0.104 | 118.87 |
| Cer(d18:1/22:0) | LQC | 1.05 | 2.7 | 1.144 | 91.72 |
| | MQC | 1.25 | 3.9 | 1.287 | 97.10 |
| | HQC | 1.87 | 5.1 | 1.859 | 100.74 |
| Cer(d18:1/24:0) | LQC | 2.95 | 1.9 | 3.112 | 94.70 |
| | MQC | 3.5 | 3.2 | 3.684 | 94.96 |
| | HQC | 3.92 | 2.7 | 4.112 | 95.27 |
| Cer(d18:1/24:1) | LQC | 1.57 | 3.7 | 1.368 | 114.46 |
| | MQC | 1.94 | 2.6 | 1.654 | 117.44 |
| | HQC | 2.36 | 4.1 | 1.94 | 121.39 |
| Cer(d20:1/24:0) | LQC | 0.0123 | 11.3 | 0.029 | 42.36 |
| | MQC | 0.0136 | 9.5 | 0.038 | 35.71 |
| | HQC | 0.0144 | 9 | 0.041 | 35.19 |
| Cer(d20:1/24:1) | LQC | 0.012 | 9.3 | 0.018 | 66.67 |
| | MQC | 0.0126 | 9.7 | 0.021 | 60.12 |
| | HQC | 0.0148 | 12.1 | 0.027 | 54.63 |
| PC(16:0/16:0) | LQC | 9.86 | 5.7 | 10.427 | 94.58 |
| | MQC | 14.5 | 1.8 | 14.999 | 96.70 |
| | HQC | 17.97 | 4.5 | 18.999 | 94.57 |
| SM(d18:1/16:0) | LQC | 157.7 | 6.5 | 152.141 | 103.65 |
| | MQC | 173.09 | 8.9 | 164.998 | 104.91 |
| | HQC | 177.2 | 8.3 | 177.855 | 99.63 |

Abbreviations: QC, quality control; CV, coefficient of variation; Cer, Ceramide; LQC, low quality control; MQC, middle quality control; HQC, high quality control; PC, phosphatidylcholine; SM, sphingomyelin.

TABLE 9
INTRA-ASSAY VARIABILITY OF ANALYTES

| Analyte | QC sample | CV (%) |
|---|---|---|
| Cer(d18:1/18:0) | LQC | 2.8 |
| | MQC | 3.4 |
| | HQC | 3 |
| Cer(d18:1/22:0) | LQC | 3 |
| | MQC | 2.1 |
| | HQC | 2.3 |
| Cer(d18:1/24:0) | LQC | 4.9 |
| | MQC | 5.7 |
| | HQC | 6.1 |
| Cer(d18:1/24:1) | LQC | 3 |
| | MQC | 1.4 |
| | HQC | 2 |
| Cer(d20:1/24:0) | LQC | 10.3 |
| | MQC | 8.6 |
| | HQC | 9 |
| Cer(d20:1/24:1) | LQC | 6.2 |
| | MQC | 11.4 |
| | HQC | 12.1 |
| PC(16:0/16:0) | LQC | 7.7 |
| | MQC | 5.6 |
| | HQC | 6.1 |
| SM(d18:1/16:0) | LQC | 4.5 |
| | MQC | 3.5 |
| | HQC | 3.7 |

Abbreviations: QC, quality control; CV, coefficient of variation; Cer, Ceramide; LQC, low quality control; MQC, middle quality control; HQC, high quality control; PC, phosphatidylcholine; SM, sphingomyelin.

### Limits of Quantification

The signal-to-noise ratio of analytes in LLOQ was greater than 20 for all lipids except for Cer(d20:1/24:1) where it was greater than 10. This was considered acceptable (refer to Table 10). The % CV for the signal-to-noise ratio of the LLOQ for PC(16:0/16:0) and SM(d18:1/16:0) was >10%. Mean LLOQ value for SM(d18:1/16:0) was below zero due to extrapolated values from the calibration curve. As the concentration of the LLOQ for these analytes was well below the target concentrations (and was much lower than any of the measured clinical samples), this was considered acceptable.

TABLE 10
LLOQ SIGNAL-TO-NOISE RATIO, MEAN
VALUE, AND COEFFICIENT OF VARIATION
ACROSS EIGHT RUN REPLICATES

| | Analyte | | | |
|---|---|---|---|---|
| | Cer | Cer | Cer | Cer |
| | (d18:1/18:0) | (d18:1/22:0) | (d18:1/24:0) | (d18:1/24:1) |
| Minimum | 20.16 | 53.21 | 54.31 | 73.58 |
| value | | | | |
| signal- | | | | |
| to-noise | | | | |
| ratio | | | | |
| Mean | 0.47 | 1.45 | 1.36 | 0.67 |
| (mg/L) | | | | |
| % CV | 0.98 | 3.96 | 9.48 | 8.66 |
| | Analyte | | | |
| | Cer | Cer | PC | SM |
| | (d20:1/24:0) | (d20:1/24:1) | (16:0/16:0) | (d18:1/16:0) |
| Minimum | 37.67 | 13.69 | 29.55 | 39.4 |
| value | | | | |
| signal- | | | | |
| to-noise | | | | |
| ratio | | | | |
| Mean | 0.09486 | 0.1936 | 1.35 | −15.88 |
| (mg/L) | | | | |
| % CV | 9.45 | 1.97 | 47.21 | −188.15 |

Abbreviations: Cer, Ceramide; PC, phosphatidylcholine; SM, sphingomyelin; % CV, coefficient of variation.

### Endogenous Lipid Concentrations in FFP-1

Table 11 shows the endogenous concentration of lipids in FFP-1 determined by the method of standard addition for the calibration curve.

TABLE 11
ENDOGENOUS CONCENTRATION (MG/L) OF EACH
LIPID SPECIES IN BLANK FFP-1 FOR EACH
OF THE EIGHT ANALYTICAL REPLICATES

| | Analyte | | | |
|---|---|---|---|---|
| | Cer | Cer | Cer | Cer |
| | (d18:1/18:0) | (d18:1/22:0) | (d18:1/24:0) | (d18:1/24:1) |
| Run #01 | 0.034 | 0.370 | 1.211 | 0.456 |
| Run #02 | 0.034 | 0.370 | 1.199 | 0.456 |
| Run #03 | 0.021 | 0.504 | 1.290 | 0.616 |
| Run #04 | 0.032 | 0.434 | 1.175 | 0.514 |
| Run #05 | 0.032 | 0.463 | 1.290 | 0.526 |
| Run #06 | 0.028 | 0.404 | 1.239 | 0.48 |
| Run #07 | 0.028 | 0.463 | 1.290 | 0.543 |
| Run #08 | 0.031 | 0.430 | 1.346 | 0.498 |
| Mean | 0.030 | 0.430 | 1.255 | 0.511 |
| concentration | | | | |
| (mg/L) | | | | |
| Standard | 0.00 | 0.05 | 0.06 | 0.05 |
| deviation | | | | |
| % CV | 14.17 | 10.97 | 4.63 | 10.32 |
| | Analyte | | | |
| | Cer | Cer | PC | SM |
| | (d20:1/24:0) | (d20:1/24:1) | (16:0/16:0) | (d18:1/16:0) |
| Run #01 | 0.009 | 0.006 | 6.200 | 99.439 |
| Run #02 | 0.009 | 0.006 | 6.200 | 99.439 |
| Run #03 | 0.009 | 0.006 | 4.988 | 77.591 |
| Run #04 | 0.013 | 0.007 | 5.772 | 99.421 |
| Run #05 | 0.002 | 0.007 | 5.114 | 60.835 |
| | (excluded) | | | (excluded) |
| Run #06 | 0.010 | 0.008 | 5.139 | 71.804 |
| Run #07 | 0.025 | 0.006 | 5.253 | 75.309 |
| Run #08 | 0.007 | 0.008 | 5.436 | 71.983 |
| Mean | 0.012 | 0.007 | 5.570 | 84.998 |
| concentration | | | | |
| (mg/L) | | | | |
| Standard | 0.01 | 0.00 | 0.49 | 13.65 |
| deviation | | | | |
| % CV | 53.44 | 13.13 | 8.83 | 16.05 |

Abbreviations: Cer, Ceramide; PC, phosphatidylcholine; SM, sphingomyelin; % CV, coefficient of variation.

### Measurement of Standard Lipids

Total cholesterol, high-density lipoprotein (HDL) and triglycerides were measured by enzymatic reaction using the COBAS 8000 analyser (module C702; Roche Diagnostics, F. Hoffmann-La Roche AG, Basel, Switzerland), with the CHOL2, Cholesterol Gen.2 (Reference No. 05168538 190* or 05168538 214*; Roche Diagnostics, F. Hoffmann-La Roche AG, Basel, Switzerland); TRIGL, triglycerides (Reference No. 05171407 190* or 05171407 214*; Roche Diagnostics, F. Hoffmann-La Roche AG, Basel, Switzerland); and HDLC4, HDL-Cholesterol Gen.4 (Reference No. 07528582 190* or 07528582 214*; Roche Diagnostics, F. Hoffmann-La Roche AG, Basel, Switzerland) assays.

### High Throughput Plasma Lipidomic Analysis Methods

Lipids were extracted from 10 μL of plasma mixed with internal standards using a butanol/methanol extraction method as described above. Pooled human plasma from healthy individuals and NIST SRM 1950 human reference plasma were extracted and analysed together with the study plasma samples as quality controls.

Lipid extracts for the discovery cohort were analysed using an Agilent 1200 liquid chromatography system combined with an applied biosystems API 4000 Q/TRAP mass spectrometer as described in Lin et al. (2017) International Journal of Cancer, 141: 2112-2120. Lipid extracts for the validation cohort were analysed using an Agilent 6490 QQQ mass spectrometer with an Agilent 1290 series HPLC system as described previously (refer to Lin et al. (2021) Prostate Cancer and Prostatic Diseases, 24:860-870; and Lin et al. (2021) EBioMedicine, 72:103625).

The concentration of lipid species was calculated by comparison with relevant internal standards and adjusted with response factors [lipid concentration=(area of analyte/area of corresponding internal standard) x concentration of internal standard x response factor].

For the discovery cohort, 323 lipid species from 22 lipid classes/subclasses were quantified, whereas for the validation cohort, 824 lipid species from 47 lipid classes were quantified.

### Dataset Normalisation, Alignment and Calculation of the 3-Lipid Signature (3LS)

To determine the presence of the circulating 3LS, both cohorts were first normalised independently according to the Probabilistic Quotient (PBQ) normalisation method as previously described Lin et al. (2017) International Journal of Cancer, 141:2112-2120. The reference sample used in PBQ normalisation was created from the mean levels of each lipid species across all the plasma samples of each cohort respectively. Final lipid levels were transformed to logarithm-2 of pmol/mL for statistical analysis.

Normalisation is a data pre-processing step that is essential for large scale analyses of multi-variable data. This normalisation step adjusts for biases that can arise from sample preparation (e.g. sample loss, evaporation, irregular extraction efficiency, pipetting errors), biological effects (e.g. differences in water content) or biological variation (e.g. differences in individuals unrelated to disease pathology).

To account for LC-MS platform and batch differences, the normalised lipidomic datasets required alignment. The validation lipidomic dataset was therefore aligned to the original cohort in Lin et al. (2017) International Journal of Cancer, 141:2112-2120 from which the 3LS was derived, using the ComBat algorithm (R package sva, v3.34.0).

Lastly, the 3LS was calculated for both cohorts as per the formula below:

$$mCRPC three - lipid signature$$

$$Patient has the three - lipid signature when P \geq 0.5$$

$$P = \frac{{e}^{y}}{1 + {e}^{y}}$$

$$y = ( 3.1319 \times ceramide ( d 18 : 1 / 24 : 1 ) ) + ( 2.1724 \times sphingomyelin ( d 18 : 2 / 16 : 0 ) ) + ( 1.8593 \times phosphatidylcholine ( 16 : 0 / 16 : 0 ) ) - 91.218$$

### Statistical Analysis

Statistical analysis was done with R software version 4.1.1 or IBM SPSS version 27. Overall survival (OS) was calculated from the date of treatment commencement to death and censored at date of last follow-up if the event had not occurred.

Pearson's correlation coefficients were used to assess the linear relationship between the lipid concentrations measured on the targeted lipid assay and those by the high-throughput lipidomic assay, or between pairs of lipid species (R package ‘ggplot2’ version 3.3.5, ‘ggpubr’ version 0.4.0). Univariable cox regression was used to determine the relationship between lipids and OS (R package ‘survival’ version 3.2-13).

Various Cox regression prognostic models of OS were built using the Discovery cohort. The least absolute shrinkage and selection operator (LASSO) method was used to select predictor variables from different combinations of lipids (R packages ‘survival’ version 3.2-13, ‘glmnet’ version 4.1-2). Unnecessary covariates were determined with the smallest value of lambda. The model with the highest concordance index (C-statistic) was considered the best model.

The sum of the variables of the cox regression formula (i.e. the lipid concentrations multiplied by the logarithm of the hazard ratio (HR) for each variable) was used as the risk score. A high score indicates high-risk (poor prognosis), whereas a low score indicates low-risk (good prognosis). To determine the optimal cut-point of the risk score that designates if a person is high-risk or low-risk, the scores between the median and worst 30^{th} percentile for the Discovery cohort were evaluated as potential cut-points. The worst 30^{th} percentile was chosen as this is the proportion of patients who had the 3LS in the Discovery cohort.

The clinical outcomes of the risk groups produced by the different cut-points were compared by univariable cox regression and Weibull regression (R package ‘survival’ version 3.2-13). The cut-point that gave the highest C-statistic and Weibull regression model log-likelihood was chosen and the performance of the model was assessed within the Validation cohort.

### Example 1—LC-MS Assay Development

Cer(d18:1/18:0), cer(d18:1/22:0), cer(d18:1/24:0), cer(d18:1/24:1), cer(d20:1/24:0), cer(d20:1/24;1), PC(16:0/16:0) and SM(d18:1/16:0) were selected as candidates for the lipid biomarker assay. A targeted LC-MS assay was developed to measure these lipids in a single run as discussed above. Total cholesterol, HDL and triglycerides were measured separately by enzymatic reaction. The LC-MS approach provided baseline separation of all lipid species (refer to FIG. 1). This assay method development meets and exceeds National Association of Testing Authorities requirements.

### Example 2-Prognostic Ability of the Targeted Lipid Biomarker Assay

Plasma samples from two cohorts of men with mCPRC were used to assess the prognostic ability of the targeted lipid biomarker assay. These cohorts, referred to as discovery and validation cohorts, consisted of 105 and 183 men respectively. The plasma lipid profiles from these men had previously been assessed via high throughput lipidomic analysis (refer to Lin et al. (2017) International Journal of Cancer, 141:2112-2120; and Lin et al. (2021) EBioMedicine, 72:103625). Patient characteristics of the cohorts are provided in Table 12. All patients in the discovery cohort were commencing treatment with taxane chemotherapy (docetaxel/cabazitaxel) whereas those in the validation cohort were commencing taxane chemotherapy or an androgen receptor signalling inhibitor (enzalutamide/abiraterone).

TABLE 12
PATIENT CHARACTERISTICS OF THE DISCOVERY
AND VALIDATION COHORTS

| | Discovery cohort | | Validation cohort | |
|---|---|---|---|---|
| Number of men | 105 | | 183 | |
| Median age, years [Q1, Q3] | 70 | [64, 76] | 73 | [66, 80] |
| Median follow-up time for | 12 | [4, 25] | 30 | [16, 46] |
| surviving patients, months [Q1, | | | | |
| Q3] | | | | |
| Median follow-up time for all | 16 | [11, 28] | 19 | [11, 31] |
| patients, months [Q1, Q3] | | | | |
| Median Overall Survival, months | 18 | [12, 34] | 22 | [12, 35] |
| [Q1, Q3] | | | | |
| Deaths | 86 | (82%) | 120 | (66%) |
| Prostate Specific Antigen at time | 128 | [48, 327] | 31 | [12, 79] |
| of sample collection, ng/mL | | | | |
| [Q1, Q3] | | | | |
| Gleason grade | | | | |
| ≤6 | 8 | (8%) | 10 | (5.46%) |
| 7 | 22 | (21%) | 35 | (19.1%) |
| 8 | 17 | (16%) | 23 | (12.6%) |
| ≥9 | 33 | (31%) | 85 | (46.4%) |
| Missing | 25 | (24%) | 30 | (16.4%) |
| Type of metastases at baseline | | | | |
| Bone | 92 | (88%) | 160 | (87%) |
| Soft tissue | 36 | (34%) | 93 | (51%) |
| Missing | 9 | (9%) | 0 | (0%) |
| Treatment | | | | |
| Docetaxel | 104 | (99%) | 48 | (26%) |
| Cabazitaxel | 1 | (1%) | 8 | (4%) |
| Enzalutamide | 0 | | 88 | (48%) |
| Abiraterone | 0 | | 39 | (21%) |
| Line of systemic treatment | | | | |
| First line | 101 | (96%) | 130 | (71%) |
| Second Line | 3 | (3%) | 53 | (29%) |
| Third Line | 1 | (1%) | 0 | (0%) |
| Subsequent lines of systemic | | | | |
| treatment | | | | |
| 0 lines | 28 | (27%) | 87 | (48%) |
| 1 line | 36 | (34%) | 57 | (31%) |
| 2 lines | 25 | (24%) | 31 | (17%) |
| 3 lines | 12 | (11%) | 8 | (4%) |
| ≥4 lines | 4 | (4%) | 0 | (0%) |
| 3-Lipid signature positive | 31 | (30%) | 69 | (38%) |

Wherein: Q1 = first quartile, Q3 = third quartile.

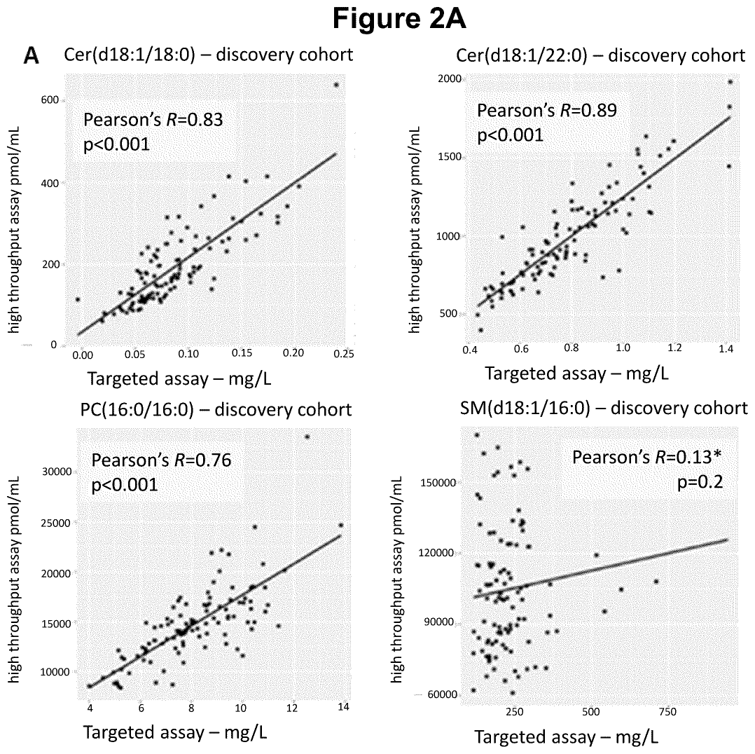

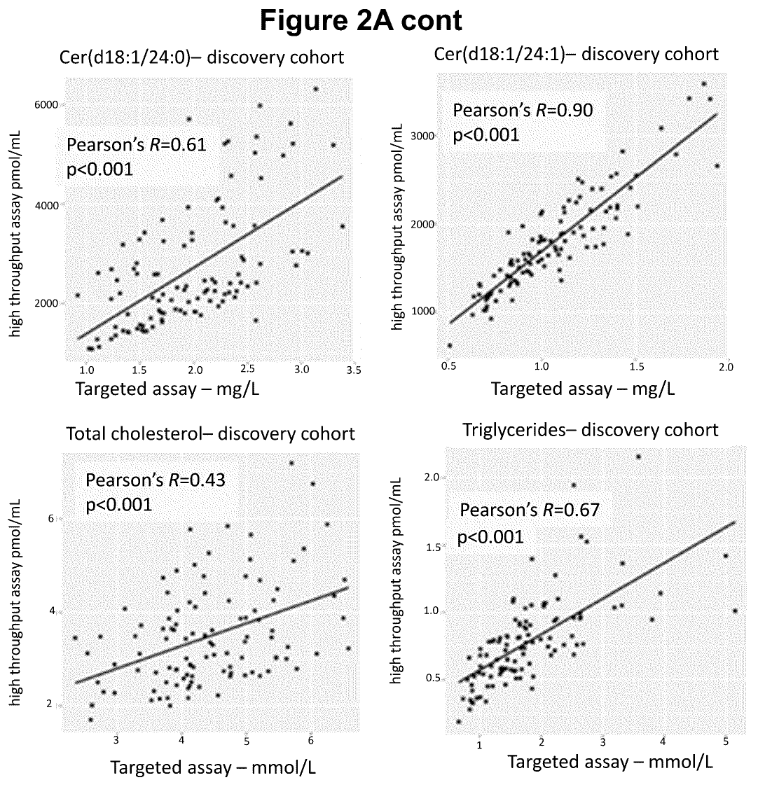

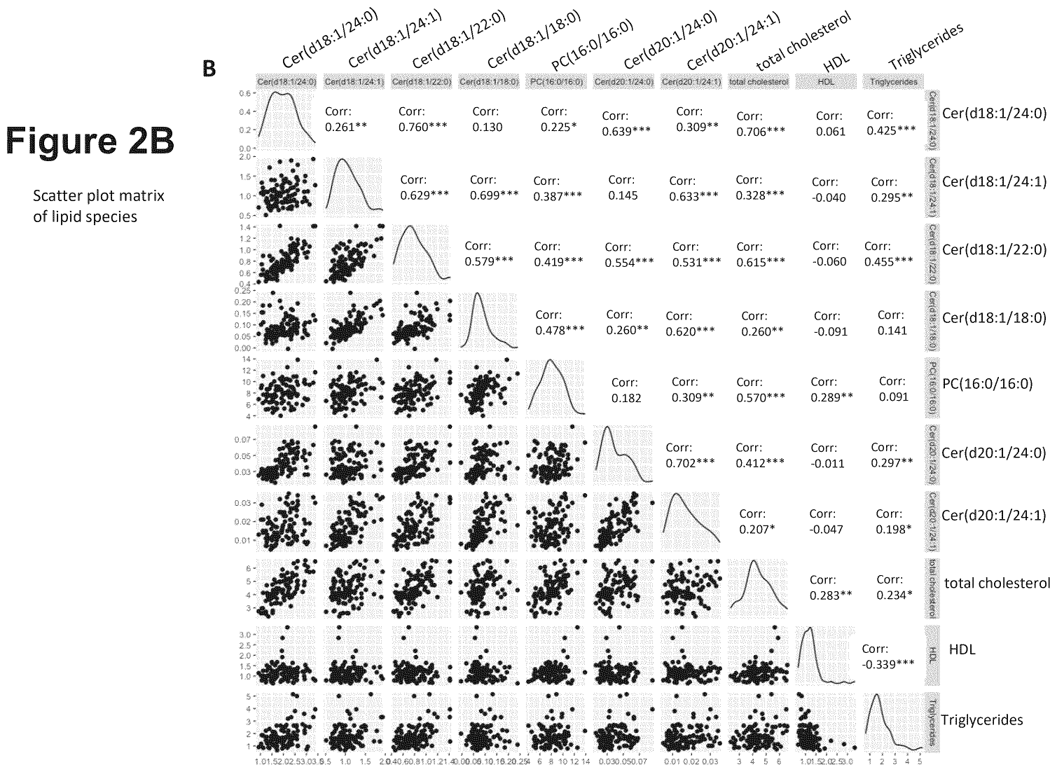

The concentration of lipids measured in plasma by the LC-MS targeted assay were highly correlated to the measurements by the high-throughput assay (Pearson's R>0.6) for all lipids except SM(d18:1/16:0) in the Discovery cohort (Pearson's R=0.13) (FIG. 2). SM(d18:1/16:0) was excluded from subsequent analyses.

None of the lipids display collinearity with each other (Pearson's R>0.75), except for Cer(d18:1/24:0) and Cer(d18:1/22:0) with Pearson's R=0.76 (S5), thus both lipids were never included in any model.

Within the Discovery cohort, four lipids measured on the high-throughput assay were associated with OS (p<0.05) (Cerd18:1/24:0), Cer(d18:1/24:1), total cholesterol and triglycerides). Similar associations were observed on the targeted assay, where Cer(d18:1/24:0), Cer(d18:1/24:1) and Cer(d20:1/24:1) were associated with OS (p<0.05) (refer to Table 13).

Within the Validation cohort, four lipids measured on the high-throughput assay were associated with OS (p<0.05) (Cerd18:1/18:0), Cer(d18:1/24:1), Cer(d20:1/24:1) and total cholesterol). Similar associations were observed with the lipids measured on the targeted assay, where Cer(d18:1/18:0), Cer(d18:1/24:0), Cer(d20:1/24:1), total cholesterol and HDL were associated with OS (p<0.05) (refer to Table 13).

TABLE 13
UNIVARIABLE COX REGRESSION OF LOG2 TRANSFORMED
LIPID SPECIES CONCENTRATIONS AS MEASURED
ON THE TARGETED AND HIGH-THROUGHPUT ASSAYS,
IN THE DISCOVERY AND VALIDATION COHORTS

| | Targeted LC-MS assay | | High-throughput lipidomics | |
|---|---|---|---|---|
| | Hazard Ratio | p- | Hazard Ratio | p- |
| Lipid | [95% CI] | value | [95% CI] | value |

DISCOVERY COHORT

| | | | | |
|---|---|---|---|---|
| Cer(d18:1/18:0) | 1.07 [0.96-1.20] | 0.2 | 1.44 [1.00-2.07] | 0.051 |
| Cer(d18:1/22:0) | 0.88 [0.48-1.60] | 0.7 | 0.88 [0.52-1.46] | 0.6 |
| Cer(d18:1/24:0) | 0.50 [0.29-0.86] | 0.013 | 0.60 [0.42-0.87] | 0.007 |
| Cer(d18:1/24:1) | 2.17 [1.18-4.02] | 0.013 | 1.92 [1.10-3.32] | 0.021 |
| Cer(d20:1/24:0) | 0.85 [0.55-1.31] | 0.5 | — | — |
| Cer(d20:1/24:1) | 1.47 [1.04-2.09] | 0.030 | — | — |
| PC(16:0/16:0) | 1.32 [0.69-2.50] | 0.4 | 1.43 [0.76-2.67] | 0.3 |
| Total cholesterol | 0.58 [0.29-1.15] | 0.12 | 0.52 [0.31-0.87] | 0.014 |
| HDL | 0.88 [0.55-1.43] | 0.6 | | |
| Triglycerides | 0.68 [0.45-1.02] | 0.062 | 0.61 [0.43-0.86] | 0.005 |

VALIDATION COHORT

| | | | | |
|---|---|---|---|---|
| Cer(d18:1/18:0) | 1.36 [1.02-1.82] | 0.037 | 1.57 [1.10-2.22] | 0.012 |
| Cer(d18:1/22:0) | 1.08 [0.87-1.33] | 0.5 | 0.97 [0.66-1.44] | 0.9 |
| Cer(d18:1/24:0) | 0.78 [0.62-0.98] | 0.032 | 0.82 [0.55-1.21] | 0.3 |
| Cer(d18:1/24:1) | 1.46 [0.92-2.32] | 0.11 | 1.62 [1.03-2.53] | 0.036 |
| Cer(d20:1/24:0) | 0.96 [0.71-1.31] | 0.8 | 0.95 [0.63-1.43] | 0.8 |
| Cer(d20:1/24:1) | 1.46 [1.06-2.01] | 0.019 | 1.50 [1.07-2.10] | 0.018 |
| PC(16:0/16:0) | 1.04 [0.59-1.85] | 0.9 | 0.95 [0.59-1.53] | 0.8 |
| Total cholesterol | 0.56 [0.34-0.94] | 0.027 | 0.54 [0.30-0.98] | 0.041 |
| HDL | 0.58 (0.39-0.86) | 0.006 | | — |
| Triglycerides | 0.95 (0.72-1.25) | 0.7 | 0.93 [0.73-1.18] | 0.6 |

Wherein: Cer, ceramide; PC, phosphatidylcholine; HDL, high-density lipoprotein; and CI, confidence interval.
Blank cells containing a “—” indicate lipids which were not measured on the high-throughput lipidomics assay

### Example 3—Model Development Using the Discovery Cohort and Performance in the Validation Cohort

Twelve models consisting of different combinations of candidate lipids were derived (refer to Table 14).

TABLE 14
DETAILS OF EACH OF THE PROGNOSTIC MODELS INVESTIGATED

| | Variables in final | | p value |
|---|---|---|---|
| Variables entered | model following | | of log- |
| into the Model⁺ | LASSO shrinkage | C-statistic | rank test |
| Model 1: | Cer(d18:1/24:0), | 0.657 | 9e−6 |
| Cer(d18:1/18:0), Cer(d18:1/22:0), | Cer(d18:1/18:0), | | |
| Cer(d18:1/24:0), Cer(d18:1/24:1), | Total cholesterol, | | |
| Cer(d20:1/24:0), Cer(d20:1/24:1), | Triglycerides | | |
| PC(16:0/16:0), total cholesterol, | | | |
| HDL, triglycerides | | | |
| Model 2: | Cer(d18:1/24:0), | 0.638 | 3e−5 |
| Cer(d18:1/18:0), Cer(d18:1/22:0), | Cer(d18:1/18:0), | | |
| Cer(d18:1/24:0), Cer(d18:1/24:1), | Cer(d20:1/24:1) | | |
| Cer(d20:1/24:0), Cer(d20:1/24:1), | | | |
| PC(16:0/16:0) | | | |
| Model 3: | Cer(d18:1/24:1), | 0.658 | 2e−5 |
| Cer(d18:1/18:0), Cer(d18:1/22:0), | Cer(d18:1/22:0), | | |
| Cer(d18:1/24:1), Cer(d20:1/24:0), | Cer(d18:1/18:0), | | |
| Cer(d20:1/24:1), PC(16:0/16:0), | Total cholesterol, | | |
| total cholesterol, HDL, triglycerides | Triglycerides | | |
| Model 4: | Total cholesterol, | 0.584 | 0.2 |
| total cholesterol, HDL, triglycerides | HDL, Triglycerides | | |
| Model 5: | Cer(d18:1/18:0), | 0.660 | 6e−6 |
| Cer(d18:1/18:0), Cer(d18:1/22:0), | Total cholesterol, | | |
| Cer(d18:1/24:0), Cer(d18:1/24:1), | Triglycerides, | | |
| Cer(d20:1/24:0), Cer(d20:1/24:1), | Cer(d18:1/24:0)/ | | |
| PC(16:0/16:0), total cholesterol, | Cer(d18:1/24:1) | | |
| HDL, triglycerides, ratio of | | | |
| Cer(d18:1/24:0)/Cer(d18:1/24:1) | | | |
| Model 6: | Cer(d18:1/18:0), | 0.660 | 8e−6 |
| Cer(d18:1/18:0), Cer(d18:1/22:0), | Total cholesterol, | | |
| Cer(d18:1/24:0), Cer(d18:1/24:1), | Triglycerides, | | |
| Cer(d20:1/24:0), Cer(d20:1/24:1), | Cer(d18:1/24:0) − | | |
| PC(16:0/16:0), total cholesterol, | Cer(d18:1/24:1) | | |
| HDL, triglycerides, difference | | | |
| between Cer(d18:1/24:0) and | | | |
| Cer(d18:1/24:1) | | | |
| Model 7: | Cer(d18:1/18:0), | Model | |
| Cer(d18:1/18:0), Cer(d18:1/22:0), | Total cholesterol, | created | |
| Cer(d18:1/24:0), Cer(d18:1/24:1), | Triglycerides, | identical | |
| Cer(d20:1/24:0), Cer(d20:1/24:1), | Cer(d18:1/24:0)/ | to Model | |
| PC(16:0/16:0), total cholesterol, | Cer(d18:1/24:1) | 5 | |
| HDL, triglycerides, ratio of | | | |
| Cer(d18:1/24:0)/Cer(d18:1/24:1), | | | |
| difference between Cer(d18:1/24:0) | | | |
| and Cer(d18:1/24:1) | | | |
| Model 8: | Cer(d18:1/18:0), | 0.653 | 2e−5 |
| Cer(d18:1/18:0), Cer(d18:1/22:0), | total cholesterol, | | |
| Cer(d18:1/24:0), Cer(d18:1/24:1), | Cer(d18:1/24:0)/ | | |
| Cer(d20:1/24:0), Cer(d20:1/24:1), | Cer(d18:1/24:1) | | |
| PC(16:0/16:0), total cholesterol, | | | |
| HDL, ratio of | | | |
| Cer(d18:1/24:0)/Cer(d18:1/24:1) | | | |
| Model 9: | Total cholesterol, | 0.653 | 3e−5 |
| Cer(d18:1/18:0), Cer(d18:1/22:0), | Cer(d18:1/18:0)/ | | |
| Cer(d18:1/24:0), Cer(d18:1/24:1), | total ceramide, | | |
| Cer(d20:1/24:0), Cer(d20:1/24:1), | Cer(d18:1/24:0)/ | | |
| PC(16:0/16:0), total cholesterol, | total ceramide | | |
| HDL, triglycerides, ratio of | | | |
| Cer(d18:1/18:0)/total ceramide, | | | |
| ratio of Cer(d18:1/22:0)/total | | | |
| ceramide**, ratio of | | | |
| Cer(d18:1/24:0)/total ceramide**, | | | |
| ratio of Cer(d18:1/24:1)/total | | | |
| ceramide**, ratio of | | | |
| Cer(d20:1/24:0)/total ceramide**, | | | |
| ratio of Cer(d20:1/24:1)/total | | | |
| ceramide** | | | |
| Model 10: | Cer(d18:1/18:0)/ | 0.648 | 1e−5 |
| Cer(d18:1/18:0), Cer(d18:1/22:0), | total ceramide, | | |
| Cer(d18:1/24:0), Cer(d18:1/24:1), | Cer(d18:1/24:0)/ | | |
| Cer(d20:1/24:0), Cer(d20:1/24:1), | Cer(d18:1/24:1) | | |
| PC(16:0/16:0), total cholesterol, | | | |
| HDL, triglycerides, ratio of | | | |
| Cer(d18:1/18:0)/total ceramide, | | | |
| ratio of Cer(d18:1/22:0)/total | | | |
| ceramide**, ratio of | | | |
| Cer(d18:1/24:0)/total ceramide**, | | | |
| ratio of Cer(d18:1/24:1)/total | | | |
| ceramide**, ratio of | | | |
| Cer(d20:1/24:0)/total ceramide**, | | | |
| ratio of Cer(d20:1/24:1)/total | | | |
| ceramide**, ratio of | | | |
| Cer(d18:1/24:0)/Cer(d18:1/24:1), | | | |
| difference between Cer(d18:1/24:0) | | | |
| & Cer(d18:1/24:1) | | | |
| Model 11: | Cer(d18:1/18:0)/ | 0.656 | 5e−6 |
| Cer(d18:1/18:0), Cer(d18:1/22:0), | total cholesterol, | | |
| Cer(d18:1/24:0), Cer(d18:1/24:1), | Cer(d18:1/24:1)/ | | |
| Cer(d20:1/24:0), Cer(d20:1/24:1), | total cholesterol, | | |
| PC(16:0/16:0), total cholesterol, | PC(16:0/16:0)/ | | |
| HDL, triglycerides, ratio of | total cholesterol | | |
| Cer(d18:1/18:0)/total cholesterol, | | | |
| ratio of Cer(d18:1/22:0)/total | | | |
| cholesterol, ratio of | | | |
| Cer(d18:1/24:0)/total cholesterol, | | | |
| ratio of Cer(d18:1/24:1)/total | | | |
| cholesterol, ratio of | | | |
| Cer(d20:1/24:0)/total cholesterol, | | | |
| ratio of Cer(d20:1/24:1)/total | | | |
| cholesterol, ratio of | | | |
| PC(16:0/16:0)/total cholesterol | | | |
| Model 12: | Total cholesterol, | 0.649 | 1e−5 |
| Cer(d18:1/18:0), Cer(d18:1/22:0), | Cer(d18:1/18:0)/ | | |
| Cer(d18:1/24:0), Cer(d18:1/24:1), | total ceramide, | | |
| Cer(d20:1/24:0), Cer(d20:1/24:1), | Cer(d18:1/18:0)/ | | |
| PC(16:0/16:0), total cholesterol, | total cholesterol, | | |
| HDL, triglycerides, ratio of | PC(16:0/16:0)/ | | |
| Cer(d18:1/18:0)/total ceramide**, | total cholesterol, | | |
| ratio of Cer(d18:1/22:0)/total | Cer(d18:1/24:0)/ | | |
| ceramide**, ratio of | Cer(d18:1/24:1) | | |
| Cer(d18:1/24:0)/total ceramide**, | | | |
| ratio of Cer(d18:1/24:1)/total | | | |
| ceramide**, ratio of | | | |
| Cer(d20: 1/24:0)/total ceramide**, | | | |
| ratio of Cer(d20:1/24:1)/total | | | |
| ceramide**, ratio of | | | |
| Cer(d18:1/18:0)/total cholesterol, | | | |
| ratio of Cer(d18:1/22:0)/total | | | |
| cholesterol, ratio of | | | |
| Cer(d18:1/24:0)/total cholesterol, | | | |
| ratio of Cer(d18:1/24:1)/total | | | |
| cholesterol, ratio of | | | |
| Cer(d20:1/24:0)/total cholesterol, | | | |
| ratio of Cer(d20:1/24:1)/total | | | |
| cholesterol, ratio of | | | |
| PC(16:0/16:0)/total cholesterol, | | | |
| ratio of | | | |
| Cer(d18:1/24:0)/Cer(d18:1/24:1), | | | |
| difference between Cer(d18:1/24:0) | | | |
| & Cer(d18:1/24:1) | | | |

Wherein: HR, hazard ratio; C-statistic, concordance; Cer, Ceramide; PC, phosphatidylcholine; and HDL, high-density lipoprotein.
⁺The values of the lipids measured by LC-MS are in mg/L. The values of lipids measured by enzymatic colorimetric assays are in mmol/L.

** Total ceramide=Cer(d18:1/18:0)+Cer(d18:1/22:0)+Cer(d18:1/24:0)+Cer(d18:1/24:1)+Cer(d20:1/24:0)+Cer(d20:1/24:1).

The formulae for each of the models post LASSO shrinkage were as follows:

$$Σβ x = ( - 0.241 \times Cer ( d 18 : 1 / 24 : 0 ) ) + ( 12.07 \times Cer ( d 18 : 1 / 18 : 0 ) ) + ( - 0.2657 \times total cholesterol ) + ( - 0.2164 \times triglycerides ) \qquad Model 1$$

$$Σβ x = ( - 0.6597 \times Cer ( d 18 : 1 / 24 : 0 ) ) + ( 9.2732 \times Cer ( d 18 : 1 / 18 : 0 ) ) + ( 10.5265 \times Cer ( d 20 : 1 / 24 : 1 ) ) \qquad Model 2$$

$$Σβ x = ( 0.4434 \times Cer ( d 18 : 1 / 24 : 1 ) ) + ( - 0.8446 \times Cer ( d 18 : 1 / 22 : 0 ) + ( 12.2 \times Cer ( d 18 : 1 / 18 : 0 ) ) + ( - 0.2957 \times total cholesterol ) + ( - 0.2128 \times triglycerides ) ) \qquad Model 3$$

$$Σβ x = ( - 0.1209 \times total cholesterol ) + ( - 0.2398 \times HDL ) + ( - 0.3013 \times triglycerides ) \qquad Model 4$$

$$Σβ x = ( 10.0506 \times Cer ( d 18 : 1 / 18 : 0 ) ) + ( - 0.2783 \times total cholesterol ) + ( - 0.224 \times triglycerides ) + ( - 0.2979 \times ( Cer ( d 18 : 1 / 24 : 0 ) / Cer ( d 18 : 1 / 24 : 1 ) ) ) \qquad Model 5$$

$$Σβ x = ( 10.9903 \times Cer ( d 18 : 1 / 18 : 0 ) ) + ( - 0.2779 \times total cholesterol ) + ( - 0.2243 \times triglycerides ) + ( - 0.2366 \times ( Cer ( d 18 : 1 / 24 : 0 ) / Cer ( d 18 : 1 / 24 : 1 ) ) ) \qquad Model 6$$

$$Σβ x = ( 10.0506 \times Cer ( d 18 : 1 / 18 : 0 ) ) + ( - 0.2783 \times total cholesterol ) + ( - 0.224 \times triglycerides ) + ( - 0.2979 \times ( Cer ( d 18 : 1 / 24 : 0 ) / Cer ( d 18 : 1 / 24 : 1 ) ) ) \qquad Model 7$$

$$Σβ x = ( 9.6096 \times Cer ( d 18 : 1 / 18 : 0 ) ) + ( - 0.2863 \times total cholesterol ) + ( - 0.3543 \times ( Cer ( d 18 : 1 / 24 : 0 ) / Cer ( d 18 : 1 / 24 : 1 ) ) ) \qquad Model 8$$

$$Σβ x = ( - 0.1579 \times total cholesterol ) + ( - 1.949 \times Cer ( d 18 : 1 / 24 : 1 ) / total ceramide ) + ( 43.75 \times Cer ( d 18 : 1 / 18 : 0 ) / \text{total ceramide} ) \qquad Model 9$$

$$Σβ x = ( 42.01 \times Cer ( d 18 : 1 / 18 : 0 ) / total ceramide ) + ( - 0.3235 \times ( Cer ( d 18 : 1 / 24 : 0 ) / Cer ( d 18 : 1 / 24 : 1 ) ) ) \qquad Model 10$$

$$Σβ x = ( 33.84 \times ( Cer ( d 18 : 1 / 18 : 0 ) / total cholesterol ) + ( 1.59 \times ( Cer ( d 18 : 1 / 24 : 1 ) / total cholestrol ) + ( 0.3304 \times ( Pc ( 16 : 0 / 16 : 0 ) / total cholestero l ) ) \qquad Model 11$$

$$Σβ x = ( - 0.1495 \times total cholesterol ) + ( 49.98 \times ( Cer ( d 18 : 1 / 18 : 0 ) / total ceramide ) ) + ( - 8.275 \times ( Cer ( d 18 : 1 / 18 : 0 ) / total cholesterol ) + ( 0.1557 \times ( PC ( 16 : 0 / 16 : 0 ) / total cholesterol ) + ( - 0.2464 \times ( Cer ( d 18 : 1 / 24 : 0 ) / Cer ( d 18 : 1 / 24 : 1 ) ) ) \qquad Model 12$$

Models 5 and 6 (models starting with all lipids plus the ratio or difference between Cer(d18:1/24:0) and Cer(d18:1/24:1) respectively) had the highest C-statistic (0.660) and were selected for further evaluation. The optimal cut-points of the risk scores that defines if a person is low- or high-risk for models 5 and 6 were −1.1903 and −0.817 respectively (refer to Tables 15 and 16).

TABLE 15
UNIVARIATE COX REGRESSION AND WEIBULL REGRESSION
MODEL RESULTS OF GROUPS FORMED BY MODEL 5 SCORES
BETWEEN THE MEDIAN AND THE WORST 30%

| | | | Weibull |
|---|---|---|---|
| | Univariate cox | | parametric |
| Model cut off | regression HR | | regression log- |
| (score for Σβx) | [95% CI] | Concordance | likelihood |
| −1.3809 (median) | 2.42 [1.55-3.77] | 0.606 | −345.0 |
| −1.3622 | 2.31 [1.48-3.59] | 0.597 | −345.8 |
| −1.3362 | 2.70 [1.70-4.27] | 0.606 | −343.3 |
| −1.3178 | 2.56 [1.62-4.05] | 0.597 | −344.2 |
| −1.2942 | 2.80 [1.76-4.46] | 0.605 | −342.9 |
| −1.2888 | 2.76 [1.74-4.38] | 0.605 | −343.3 |
| −1.2828 | 2.96 [1.85-4.73] | 0.611 | −342.2 |
| −1.281 | 2.96 [1.85-4.71] | 0.613 | −342.4 |
| −1.2711 | 2.89 [1.82-4.60] | 0.612 | −343.0 |
| −1.2607 | 2.88 [1.81-4.58] | 0.613 | −343.2 |
| −1.2340 | 2.93 [1.84-4.66] | 0.616 | −343.2 |
| −1.2293 | 2.97 [1.87-4.73] | 0.618 | −343.3 |
| −1.2219 | 3.03 [1.90-4.84] | 0.621 | −343.2 |
| −1.2198 | 3.72 [2.27-6.10] | 0.629 | −340.3 |
| −1.1953 | 3.73 [2.28-6.12] | 0.630 | −340.2 |
| −1.1903** | 3.75 [2.29-6.15] | 0.631 | −340.1 |
| −1.1885 | 3.67 [2.24-6.01] | 0.629 | −340.6 |
| −1.181 | 3.60 [2.20-5.89] | 0.626 | −341.2 |
| −1.1708 | 3.61 [2.21-5.90] | 0.627 | −341.2 |
| −1.1669 | 3.71 [2.26-6.08] | 0.629 | −341.2 |
| −1.166 (worst 30%) | 3.80 [2.31-6.26] | 0.630 | −341.3 |

**optimal cut-off.

TABLE 16
UNIVARIATE COX REGRESSION AND WEIBULL REGRESSION
MODEL RESULTS OF GROUPS FORMED BY MODEL 6 SCORES
BETWEEN THE MEDIAN AND THE WORST 30%

| | | | | Weibull |
|---|---|---|---|---|
| | Univariate cox | | | parametric |
| Model cut off | regression HR | | | regression log- |
| (score for Σβx) | [95% CI] | | Concordance | likelihood |
| −0.926 (median) | 2.74 | [1.72-4.35] | 0.603 | −343.1 |
| −0.914 | 2.77 | [1.74-4.39] | 0.608 | −342.8 |
| −0.913 | 3.03 | [1.90-4.85] | 0.615 | −341.3 |
| −0.912 | 2.98 | [1.87-4.75] | 0.616 | −341.7 |
| −0.906 | 2.97 | [1.87-4.72] | 0.617 | −342.0 |
| −0.861 | 2.99 | [1.89-4.76] | 0.620 | −342.0 |
| −0.8397 | 2.85 | [1.79-4.51] | 0.611 | −343.0 |
| −0.8396 | 2.78 | [1.76-4.41] | 0.610 | −343.5 |
| −0.832 | 3.00 | [1.88-4.78] | 0.616 | −342.4 |
| −0.8238 | 3.01 | [1.89-4.79] | 0.617 | −342.3 |
| −0.8237 | 3.05 | [1.92-4.86] | 0.620 | −342.2 |
| −0.817** | 3.71 | [2.26-6.08] | 0.628 | −339.3 |
| −0.808 | 3.63 | [2.21-5.94] | 0.627 | −339.9 |
| −0.785 | 3.54 | [2.16-5.78] | 0.624 | −340.5 |
| −0.766 | 3.51 | [2.15-5.73] | 0.624 | −341.0 |
| −0.755 | 3.53 | [2.16-5.77] | 0.625 | −341.2 |
| −0.77 | 3.65 | [2.23-5.97] | 0.627 | −341.2 |
| −0.762 | 3.63 | [2.21-5.95] | 0.626 | −341.7 |
| −0.76 | 3.64 | [2.22-5.97] | 0.627 | −341.5 |
| −0.757 | 3.58 | [2.18-5.87] | 0.625 | −342.1 |
| −0.75 (worst 30%) | 3.672 | [2.23-6.04] | 0.626 | −342.2 |

**Optimal cut-off.

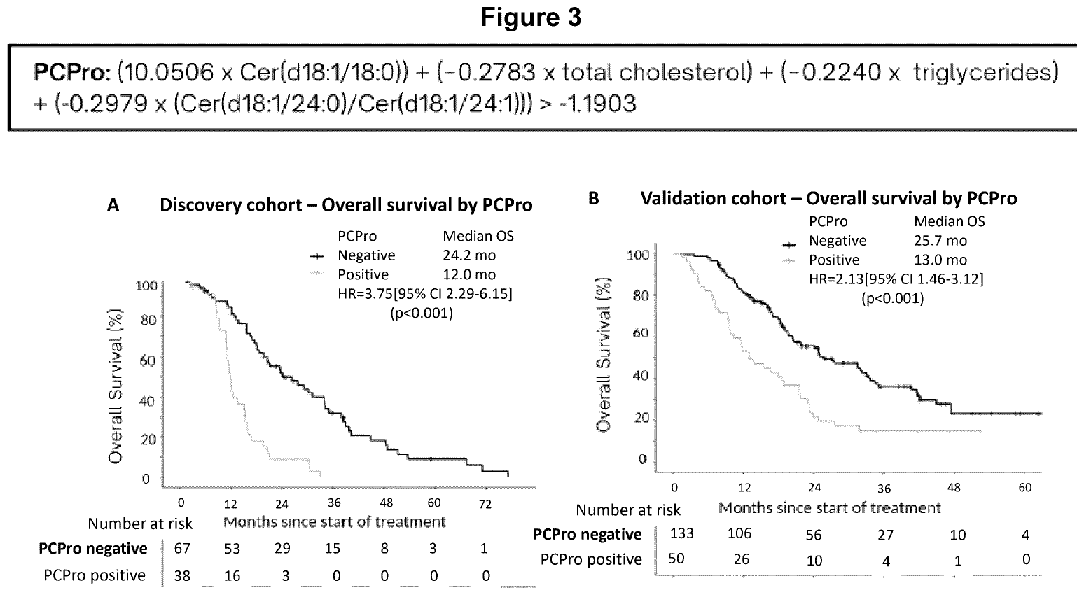

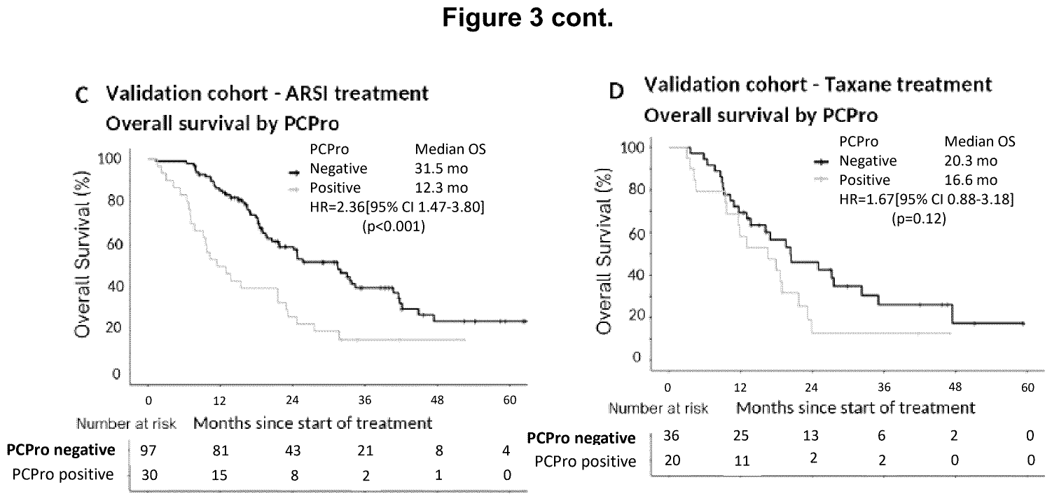

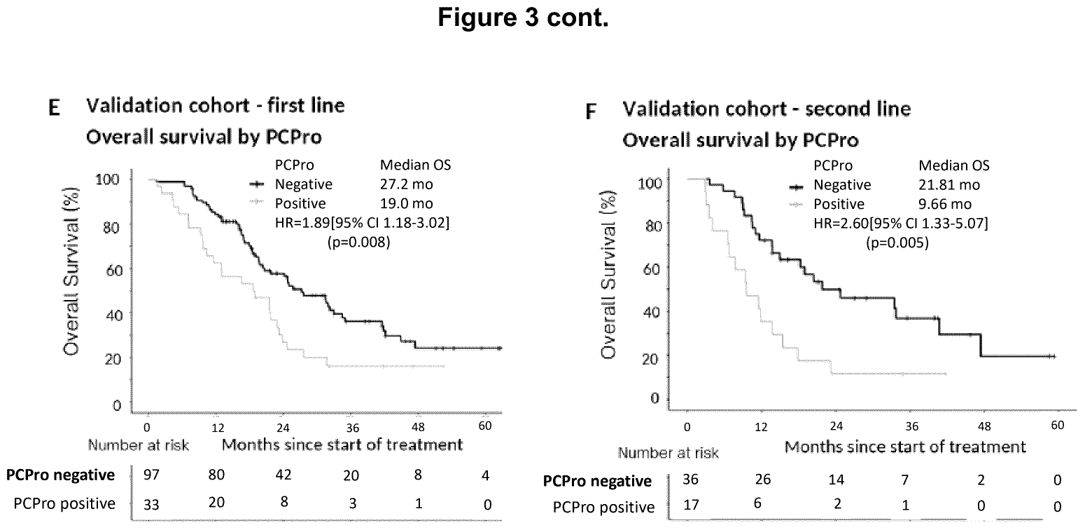

Using both models, the poor-risk groups had significantly shorter OS than the low-risk groups (model 5: median OS of 12.0 months vs. 24.2 months, HR 3.75 [95% confidence interval (CI) 2.29-6.15], p<0.001; model 6: median OS of 12.2 months vs. 26.4 months, HR 3.62 [2.21-5.94], p<0.001) (refer to FIG. 3). Model 5 was chosen as the optimal model.

The performance of model 5 (hereinafter termed “PCPro”) in the validation cohort was then assessed. Within the validation cohort, the median OS for those who were PCPro positive was significantly shorter than the PCPro negative group (13.0 months vs 25.7 months, HR=2.13 [95% CI 1.46-3.12], log-rank p<0.001) (refer to FIG. 3).

For those treated with an ARSI, median OS was significantly shorter in the PCPro positive group compared to the PCPro negative group (12.3 months vs 31.5 months, HR=2.36 [1.47-3.80], log-rank p<0.001). Although OS was shorter amongst those treated with taxanes who were PCPro positive compared to PCPro negative, it did not reach statistical significance (median OS 16.6 months vs 20.3 months, HR=1.67 [0.88-3.18], log-rank p=0.12) (refer to FIG. 3).

Men within the PCPro positive group had shorter OS when treated with either first- or second-line treatment (first line: median OS 19.0 months vs 27.2 months, HR=1.89 [1.18-3.02], log-rank p=0.008; second line: median OS 9.66 months vs 21.81 months, HR=2.60 [1.33-5.07], log-rank p=0.005) (refer to FIG. 3).

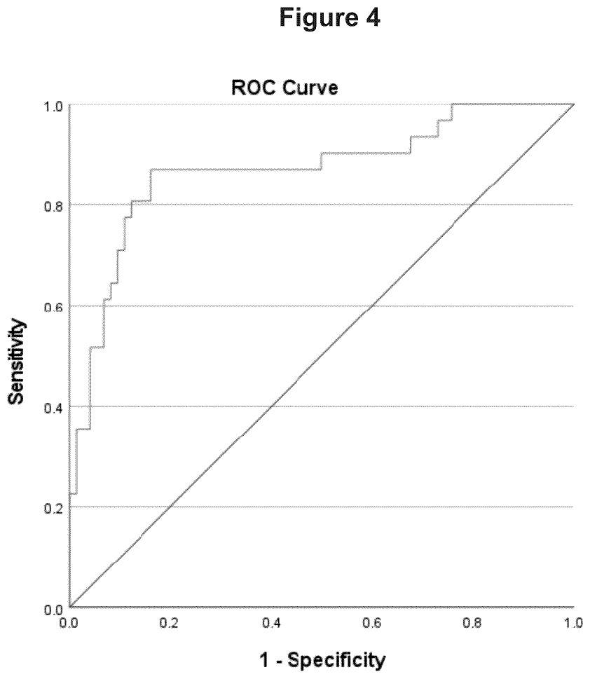

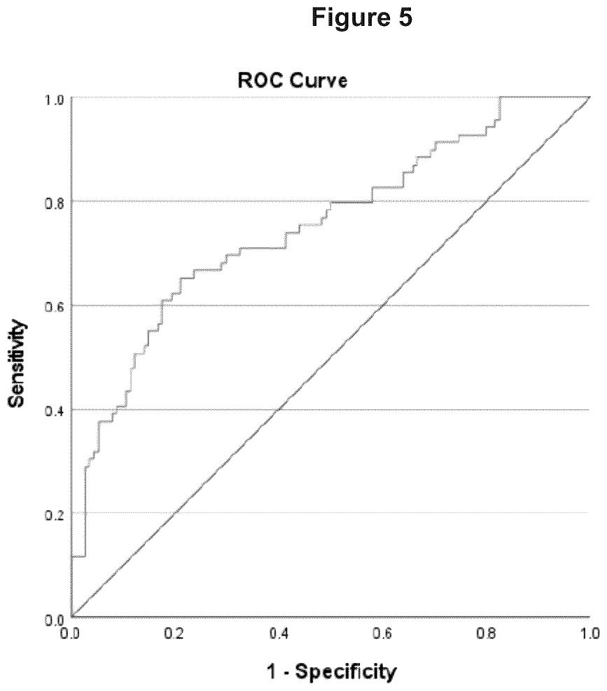

PCPro correlated well with the three-lipid signature (3LS) previously described, with a Receiver Operating Characteristic (ROC) area under the curve (AUC) of 0.869 in the discovery cohort and 0.751 in the validation cohort (refer to FIGS. 4 and 5 and Tables 17 and 18).

TABLE 17
COX REGRESSION AND SURVIVAL ANALYSIS OF
3LS AND PCP_{RO} IN THE DISCOVERY COHORT

| | | | | | Cox regression of risk | |
|---|---|---|---|---|---|---|
| | | | | | groups | |
| | | | Survival analysis | | | p-value |
| Model | | | Median overall | | | (log- |
| (discovery | Risk | | survival, | p- | | rank |
| cohort) | group | Cases | months | value | HR [95% CI] | test) |
| 3LS | Low risk | 74 | 23.7 | <0.001 | 3.94 | <0.001 |
| | High risk | 31 | 11.5 | | [2.38-6.52] | |
| PCPro | Negative | 64 | 26.4 | <0.001 | 3.63 | <0.001 |
| | Positive | 41 | 12.2 | | [2.21-5.94] | |

TABLE 18
Cox REGRESSION AND SURVIVAL ANALYSIS OF
3LS AND PCP_{RO} IN THE VALIDATION COHORT

| | | | Survival analysis | | Cox regression of risk | |
|---|---|---|---|---|---|---|
| | | | Median | | groups | |
| Model | | | overall | | | p-value |
| (vali- | | | sur- | | | (log- |
| dation | Risk | | vival, | p- | | rank |
| cohort) | group | Cases | months | value | HR [95% CI] | test) |
| 3LS | Low risk | 114 | 25.0 | <0.001 | 1.96 | <0.001 |
| | High risk | 69 | 16.1 | | [1.34-2.85] | |
| PCPro | Negative | 131 | 27.2 | <0.001 | 2.38 | <0.001 |
| | Positive | 52 | 13.8 | | [1.60-3.53] | |

When PCPro was modelled with clinicopathological factors in multivariate cox regression, PCPro and haemoglobin were independent predictors of OS (p<0.05) in both the discovery and validation cohorts, and alkaline phosphatase (ALP) was an independent predictor in the discovery cohort alone (refer to Tables 19-23). Diabetes was not significantly correlated with PCPro, nor was it a predictor of OS in the discovery cohort (refer to Tables 24 and 25). Data on diabetic status was not available for the validation cohort.

TABLE 19
COX REGRESSION ANALYSIS

| | Univariate analysis | | Multivariable analysis | |
|---|---|---|---|---|
| | Hazard ratio | P-value | Hazard ratio | |
| Variable* | [95% CI] | (log-rank test) | [95% CI] | P-value |

Discovery Cohort

| | | | | |
|---|---|---|---|---|
| PCPro (positive vs. | 3.75 [2.29-6.15] | <0.001*** | 3.19 [1.90-5.35] | <0.001*** |
| negative) | | | | |
| Alkaline | 1.00 [1.00-1.00] | 0.005*** | 1.00 [1.00-1.00] | 0.025*** |
| phosphatase | | | | |
| Haemoglobin | 0.98 [0.96-0.99] | 0.003*** | 0.99 [0.97-1.00] | 0.075 |
| Serum PSA | 1.00 [1.00-1.00] | 0.6 | | |
| Metastasis site | 1.17 [0.75-1.82] | 0.5 | | |
| (bone only/none vs. | | | | |
| soft tissue)** | | | | |

Validation Cohort

| | | | | |
|---|---|---|---|---|
| PCPro (positive vs. | 2.37 [1.59-3.52] | <0.001*** | 2.10 [1.37-3.23] | <0.001*** |
| negative) | | | | |
| Alkaline | 1.00 [1.00-1.00] | 0.03*** | 1.00 [1.00-1.00] | 0.6 |
| phosphatase | | | | |
| Haemoglobin | 0.98 [0.97-0.99] | <0.001*** | 0.98 [0.97-0.99] | <0.001*** |
| Serum PSA | 1.00 [1.00-1.00] | 0.05*** | 1.00 [1.00-1.00] | 0.5 |
| Metastasis site | 1.46 [0.78-2.73] | 0.2 | | |
| (bone only, | | | | |
| lymph node, none | | | | |
| vs. visceral) | | | | |
| Neutrophil to | 1.04 [0.99-1.08] | 0.1 | | |
| lymphocyte ratio | | | | |

*Analysed as a continuous variable unless stated otherwise
**Visceral and nodal metastases were not distinguished in our database in the discovery cohort
***P value statistically significant (p ≤ 0.05)
Abbreviations: CI = confidence interval, PSA = Prostate specific antigen.

Halabi's nomogram was accessed at: https://www.cancer.duke.edu/Nomogram/firstlinechemotherapy.html. Some of the clinical variables for Halabi's nomogram were unavailable (discovery cohort: lactate dehydrogenase levels unavailable for all patients; ECOG performance status, albumin levels, and opiate usage only available for some patients; validation cohort: data on opiate usage unavailable for all patients; lactate dehydrogenase levels, ECOG performance status, albumin levels only available for some patients). Missing values were estimated as described in the table footnotes below. For additional comparison utilising complete clinicopathological data, another clinicopathological model was derived from multivariable Cox regression of existing data, also described in the table footnotes below.

TABLE 20
SURVIVAL ANALYSIS OF RISK GROUPS IN THE
DISCOVERY COHORT USING HALABI'S NOMOGRAM

| | | | | | Cox regression of risk | |
|---|---|---|---|---|---|---|
| | | | | | groups | |
| | | | Survival analysis | | | p- |
| | | | Median | | | value |
| | | | overall | | | (log- |
| Model (discovery | Risk | | survival, | | | rank |
| cohort) | group | Cases | months | p-value | HR [95% CI] | test) |
| Halabi's | Low risk | 32 | 27.8 | 0.010 | 1.94 | 0.009 |
| nomogram * | High risk | 70 | 15.8 | | [1.17-3.20] | |
| Clinicopathological | Low risk | 66 | 21.2 | 0.002 | 2.10 | 0.001 |
| model (ALP, Hb, | High risk | 37 | 12.2 | | [1.33-3.31] | |
| PSA, mets)** | | | | | | |
| PCPro | Negative | 67 | 24.2 | <0.001 | 3.75 | <0.001 |
| | Positive | 38 | 12.0 | | [2.29-6.15] | |

* Lactate dehydrogenase values were unavailable and thus were assumed to be below the normal limit for all patients. Missing values for ECOG performance status, albumin levels and opiate use were estimated by the k-nearest neighbours method with an Euclidean metric, using the module “ImputeMissingValuesKNN” in GenePattern version 3.2.3. Number of neighbours used in the imputation was 10.
**The clinicopathological model is the prognostic index (B₁X₁ + B₂X₂ + B₃X₃ + . . . + BₙXₙ) of multivariable Cox regression of alkaline phosphatase (ALP), haemoglobin (Hb), PSA, and metastasis site (mets) (see below). The size of the risk groups are based on the same proportion as for PCPro.

TABLE 21
REGRESSION COEFFICIENTS OF MULTIVARIABLE
COX REGRESSION OF CLINICOPATHOLOGICAL
FACTORS OF THE DISCOVERY COHORT (103 CASES)
FOR THE CLINICOPATHOLOGICAL MODEL

| | Regression | | |
|---|---|---|---|
| Variable | coefficient (B) | HR [95% CI] | p-value |
| Alkaline | 0.00056620 | 1.00 [1.00-1.00] | 0.020 |
| phosphatase (U/L) | | | |
| Haemoglobin (U/L) | −0.0246 | 0.98 [0.96-0.99] | 0.006 |
| PSA (μg/L) | −0.00001845 | 1.00 [1.00-1.00] | 0.8 |
| Metastatic site | 0.1421 | 1.15 [0.73-1.81] | 0.5 |
| (bone only/none vs | | | |
| soft tissue)* | | | |

*Nodal metastases were not distinguished from visceral metastases in the database.

TABLE 22
SURVIVAL ANALYSIS OF RISK GROUPS IN THE
VALIDATION COHORT USING HALABI'S NOMOGRAM

| | | | | | Cox regression of risk | |
|---|---|---|---|---|---|---|
| | | | | | groups | |
| | | | Survival analysis | | | p- |
| | | | Median | | | value |
| | | | overall | | | (log- |
| Model (validation | Risk | | survival, | | | rank |
| cohort) | group | Cases | months | p-value | HR [95% CI] | test) |
| Halabi's | Low risk | 105 | 27.2 | <0.001 | 2.29 | <0.001 |
| nomogram* | High risk | 78 | 13.8 | | [1.57-3.34] | |
| Clinicopathological | Low risk | 133 | 24.7 | <0.001 | 1.62 | 0.021 |
| model (ALP, Hb, | High risk | 50 | 16.6 | | [1.08-2.43] | |
| PSA, mets)** | | | | | | |
| PCPro | Negative | 133 | 25.7 | <0.001 | 2.37 | <0.001 |
| | Positive | 50 | 13.0 | | [1.59-3.52] | |

*Opioid usage data was unavailable and was assumed to be “no usage”. Lactate dehydrogenase values were only available for some patients, and were assumed to be below the normal limit for all other patients. Missing values for ECOG performance status and albumin levels were estimated by the k-nearest neighbours method with an Euclidean metric, using the module “ImputeMissingValuesKNN” in GenePattern version 3.2.3. Number of neighbours used in the imputation was 10.
**The clinicopathological model is the prognostic index (B₁X₁ + B₂X₂ + B₃X₃ + . . . + BₙXₙ) of multivariable Cox regression of alkaline phosphatase (ALP), haemoglobin (Hb), PSA, and metastasis site (mets) (see below). The size of the risk groups are based on the same proportion as for PCPro

TABLE 23
REGRESSION COEFFICIENTS OF MULTIVARIABLE
COX REGRESSION OF CLINICOPATHOLOGICAL
FACTORS OF THE VALIDATION COHORT FOR
THE CLINICOPATHOLOGICAL MODEL

| | Regression | | |
|---|---|---|---|
| Variable | coefficient (B) | HR [95% CI] | p-value |
| Alkaline | 0.0001905 | 1.00 [1.00-1.00] | 0.1 |
| phosphatase (U/L) | | | |
| Haemoglobin (U/L) | −0.0207306 | 0.98 [0.97-0.99] | <0.001 |
| PSA (μg/L) | 0.0002675 | 1.00 [1.00-1.00] | 0.2 |
| Metastatic site | 0.2914266 | 1.34 [0.71-2.52] | 0.4 |
| (bone only, lymph | | | |
| node, none vs. | | | |
| visceral) | | | |
| Neutrophil to | 0.0199467 | 1.02 [0.98-1.07] | 0.4 |
| lymphocyte ratio | | | |

TABLE 24
RELATIONSHIP BETWEEN PCPRO AND DIABETIC
STATUS IN THE DISCOVERY COHORT

| | | Number of cases (%) | | |
|---|---|---|---|---|
| | | PCPro | PCPro | Chi-square |
| Discovery cohort | | negative | positive | test, p-value |
| Diabetes | Diagnosed | 10 | 5 | 0.5 |
| status* | Not | 50 | 36 | |
| | diagnosed | | | |

*Diabetes status unavailable for 4 patients (all PCPro negative)

TABLE 25
COX REGRESSION ANALYSIS FOR DIABETES

| | Univariable cox | | Multivariable cox | |
|---|---|---|---|---|
| | regression | | regression | |
| | Hazard ratio | p-value | Hazard ratio | |
| Variable* | [95% CI] | (log-rank test) | [95% CI] | p-value |
| Diabetes | 1.08 [0.57-2.05] | 0.8 | 1.13 [0.59-2.15] | 0.7 |
| (diagnosed/not diagnosed) | | | | |
| PCPro | 3.79 [2.29-6.28] | <0.001 | 3.80 [2.29-6.30] | <0.001 |
| (positive/negative) | | | | |

*Only included patients who have diabetic status available

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

The present application claims priority from Australian provisional application no. 2022902527, the entirety of which is incorporated herein by reference.

1. A method for treating or inhibiting the progression of prostate cancer in a subject, comprising, consisting or consisting essentially of:
(a) identifying a subject with prostate cancer having a poor prognosis using an indicator used in determining prognosis for a subject with prostate cancer, the indicator determined by a method comprising, consisting or consisting essentially of:
(i) determining the level of at least three lipid biomarkers in a biological sample obtained from the subject, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0); and
(ii) determining the indicator using the biomarker levels; and
(b) administering a treatment for the prostate cancer.

2. The method of claim 1, wherein the indicator used in determining prognosis is used in determining a likelihood a subject with prostate cancer is resistant to conventional treatment with chemotherapy or treatment with an androgen receptor signalling inhibitor.

3. (canceled)

4. The method according to claim 1, wherein the prostate cancer is a metastatic cancer.

5.-6. (canceled)

7. The method according to claim 1, wherein the lipid biomarkers are ceramide (d18:1/18:0), ceramide (d18:1/24:0) and ceramide (d18:1/24:1).

8. The method according to claim 1, wherein step a) further comprises determining the level of one or more biomarkers selected from the group consisting of total cholesterol, triglycerides and high-density lipoprotein.

9. (canceled)

10. The method according to claim 1, wherein step a) further comprises determining the level of total cholesterol and triglycerides.

11. The method according to claim 1, wherein step a) comprises determining the level of ceramide (d18:1/18:0), ceramide (d18:1/24:0), ceramide (d18:1/24:1), total cholesterol and triglycerides.

12. The method according to claim 1, wherein the biological sample is blood, serum or plasma.

13. (canceled)

14. The method according to claim 1, wherein a poor prognosis comprises disease progression and/or death from disease within about 18 months from commencement of chemotherapy or treatment with an androgen receptor signalling inhibitor, and wherein the indicator indicates a likelihood of a poor prognosis if:
i. the level of ceramide (d18:1/18:0) is higher than in control biological samples obtained from a reference population of subjects with prostate cancer with a favourable outcome;
ii. the level of ceramide (d18:1/22:0) is lower than in control biological samples obtained from a reference population of subjects with prostate cancer with a favourable outcome;
iii. the level of ceramide (d18:1/24:0) is lower than in control biological samples obtained from a reference population of subjects with prostate cancer with a favourable outcome;
iv. the level of ceramide (d18:1/24:1) is higher than in control biological samples obtained from a reference population of subjects with prostate cancer with a favourable outcome;
V. the level of ceramide (d20:1/24:0) is lower than in control biological samples obtained from a reference population of subjects with prostate cancer with a favourable outcome;
vi. the level of ceramide (d20:1/24:1) is higher than in control biological samples obtained from a reference population of subjects with prostate cancer with a favourable outcome; and/or
vii. the level of phosphatidylcholine (16:0/16:0) is higher than in control biological samples obtained from a reference population of subjects with prostate cancer with a favourable outcome.

15.-16. (canceled)

17. The method according to claim 1, wherein step a) further comprises determining the level of one or more biomarkers selected from the group consisting of total cholesterol, triglycerides and high-density lipoprotein, and the indicator indicates a likelihood of a poor prognosis if:
i. the level of total cholesterol is lower than in control biological samples obtained from a reference population of subjects with prostate cancer with a favourable outcome;
ii. the level of triglycerides is lower than in control biological samples obtained from a reference population of subjects with prostate cancer with a favourable outcome; and/or
iii. the level of high-density lipoprotein is lower than in control biological samples obtained from a reference population of subjects with prostate cancer with a favourable outcome.

18. The method according to claim 1, further comprising applying a function to biomarker levels to yield at least one functionalised biomarker level and determining the indicator using the at least one functionalised biomarker level, wherein the function includes at least one of: (a) multiplying biomarker levels; (b) dividing biomarker levels; (c) adding biomarker levels; and (d) subtracting biomarker levels.

19. (canceled)

20. The method according to claim 1, further comprising combining the biomarker levels and/or functionalised biomarker levels to provide a composite score and determining the indicator using the composite score, wherein the biomarker levels and/or functionalised biomarker levels are combined by adding, multiplying, subtracting, and/or dividing biomarker levels and/or functionalised biomarker levels.

21. (canceled)

22. The method according to claim 1, further comprising analysing the biomarker levels, functionalised biomarker level(s) or composite score with reference to a corresponding reference biomarker level range or cut-off levels, functionalised biomarker level range or cut-off levels, or reference composite score range or cut-off scores, to determine the indicator.

23. The method according to claim 1, wherein the method comprises determining the indicator by dividing the level of ceramide (d18:1/24:0) by the level of ceramide (d18:1/24:1), and adding this value to the level of ceramide (d18:1/18:0), the level of total cholesterol, and the level of triglycerides, or functionalised levels of any of the foregoing levels.

24. The method according to claim 1, wherein the method comprises determining the indicator using Formula I:
[10.0506×the level of ceramide (d18:1/18:0) in mg/L]+[−0.2783×the level of total cholesterol in mmol/L]+[−0.2240×the level of triglycerides in mmol/L]+[−0.2979×(the level of ceramide (d18:1/24:0) in mg/L/the level of ceramide (d18:1/24:1) in mg/L)] Formula I.

25. The method according to claim 24, wherein a score of greater than or equal to about −0.5 to about −2 calculated using Formula I indicates a likelihood of a poor prognosis.

26.-27. (canceled)

28. The method according to claim 1, wherein a subject determined to have a poor prognosis is administered a treatment for prostate cancer which comprises administering a lipid targeted therapy.

29. (canceled)

30. The method according to claim 28, wherein the treatment comprises administering evolocumab or opaganib.

31.-33. (canceled)

34. A method for treating or inhibiting the progression of prostate cancer in a subject, comprising, consisting or consisting essentially of:
(a) identifying a subject with prostate cancer having a poor prognosis using a method for determining or predicting prognosis for a subject with prostate cancer, the method comprising, consisting or consisting essentially of:
(i) determining the level of at least three lipid biomarkers in a biological sample obtained from the subject, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0);
(ii) comparing the level of the biomarkers to the respective levels of the corresponding biomarkers in a reference sample; and
(iii) determining or predicting prognosis for the subject based on the determined levels of the biomarkers and said comparison; and
(b) administering a treatment for the prostate cancer.

35. The method of claim 34, wherein the method for determining or predicting prognosis for a subject with prostate cancer determines a likelihood a subject is resistant to conventional treatment with chemotherapy or treatment with an androgen receptor signalling inhibitor.

36. The method according to claim 34, wherein the reference sample is a control biological sample obtained from a reference population of subjects with prostate cancer having a favourable outcome.

37.-38. (canceled)

39. The method according to claim 34, wherein the prostate cancer is a metastatic cancer.

40.-41. (canceled)

42. The method according to claim 34, wherein the lipid biomarkers are ceramide (d18:1/18:0), ceramide (d18:1/24:0) and ceramide (d18:1/24:1).

43.-44. (canceled)

45. The method according to claim 34, wherein step a) further comprises determining the level of total cholesterol and triglycerides.

46. The method according to claim 34, wherein step a) comprises determining the level of ceramide (d18:1/18:0), ceramide (d18:1/24:0), ceramide (d18:1/24:1), total cholesterol and triglycerides.

47. The method according to claim 34, wherein the biological sample is blood, serum or plasma.

48.-50. (canceled)

51. The method according to claim 34, wherein the reference sample is a control biological sample obtained from a reference population of subjects with prostate cancer having a favourable outcome, and the subject has a likelihood of a poor prognosis if:
i. the level of ceramide (d18:1/18:0) is higher than in the reference sample;
ii. the level of ceramide (d18:1/22:0) is lower than in the reference sample;
iii. the level of ceramide (d18:1/24:0) is lower than in the reference sample;
iv. the level of ceramide (d18:1/24:1) is higher than in the reference sample;
V. the level of ceramide (d20:1/24:0) is lower than in the reference sample;
vi. the level of ceramide (d20:1/24:1) is higher than in the reference sample; and/or
vii. the level of phosphatidylcholine (16:0/16:0) is higher than in the reference sample.

52. The method according to claim 51, wherein step a) further comprises determining the level of one or more biomarkers selected from the group consisting of total cholesterol, triglycerides and high-density lipoprotein, the reference sample is a control biological sample obtained from a reference population of subjects with prostate cancer having a favourable outcome and the subject has a likelihood of a poor prognosis if:
i. the level of total cholesterol is lower than in the reference sample;
ii. the level of triglycerides is lower than in the reference sample; and/or
iii. the level of high-density lipoprotein is lower than in the reference sample.

53. The method according to claim 34, wherein the method comprises determining the prognosis for the subject by dividing the level of ceramide (d18:1/24:0) by the level of ceramide (d18:1/24:1), and adding this value to the level of ceramide (d18:1/18:0), the level of total cholesterol, and the level of triglycerides, or functionalised levels of any of the foregoing levels.

54. The method according to claim 34, wherein the method comprises determining the prognosis for the subject using Formula I:
[10.0506×the level of ceramide (d18:1/18:0) in mg/L]+[−0.2783×the level of total cholesterol in mmol/L]+[−0.2240×the level of triglycerides in mmol/L]+[−0.2979×(the level of ceramide (d18:1/24:0) in mg/L/the level of ceramide (d18:1/24:1) in mg/L)] Formula I.

55. The method according to claim 54, wherein a score of greater than or equal to about −0.5 to about −2 calculated using Formula I indicates a likelihood of a poor prognosis.

56.-58. (canceled)

59. The method according to claim 34, wherein the treatment comprises administering a lipid targeted therapy.

60. The method according to claim 59, wherein the treatment comprises administering evolocumab or opaganib.

61.-132. (canceled)

133. A kit for determining or predicting prognosis for a subject with prostate cancer, comprising one or more reagents for determining the level of at least three lipid biomarkers in a biological sample from the subject, wherein the at least three lipid biomarkers are selected from the group consisting of ceramide (d18:1/18:0), ceramide (d18:1/22:0), ceramide (d18:1/24:0), ceramide (d18:1/24:1), ceramide (d20:1/24:0), ceramide (d20:1/24:1), phosphatidylcholine (16:0/16:0) and sphingomyelin (d18:1/16:0).

134.-168. (canceled)
